(19) United States
(12) Patent Application Publication
(10) Pub. No.: US 2024/0294863 A1
(43) Pub. Date: Sep. 5, 2024

(54) AUTONOMOUS MAINTENANCE AND DIFFERENTIATION OF INDUCED PLURIPOTENCY CELLS

(71) Applicant: Insitro, Inc., South San Francisco, CA (US)

(72) Inventors: Brigham HARTLEY, Melbourne (AU); Haoyang ZENG, Redwood City, CA (US); Joseph Anthony MARRAMA, San Francisco, CA (US); David CONEGLIANO, South San Francisco, CA (US); Kelly Marie HASTON, Pacifica, CA (US); Lauren SCHIFF, San Mateo, CA (US); Matthew CHEN, Palo Alto, CA (US)

(73) Assignee: Insitro, Inc., South San Francisco, CA (US)

(21) Appl. No.: 18/592,877

(22) Filed: Mar. 1, 2024

Related U.S. Application Data
(60) Provisional application No. 63/449,421, filed on Mar. 2, 2023.

(51) Int. Cl. C12M 1/36; C12M 1/00; C12M 1/32; C12M 1/34

(52) U.S. Cl. CPC C12M 41/48; C12M 23/12; C12M 41/36; C12M 47/02; C12M 47/04

(57) ABSTRACT

The present disclosure relates to an autonomous system for maintaining and differentiating induced pluripotency cells (iPSCs) based on quality and confluence conditions using machine learning, to obtain differentiated cells for phenotypic analyses and/or other cellular assays.

### CROSS-REFERENCE TO RELATED APPLICATION

This application claims the benefit of U.S. Provisional Application No. 63/449,421 filed on Mar. 2, 2023, the entire content of which is incorporated herein by reference for all purposes.

### FIELD

The present disclosure relates generally to an autonomous system for maintaining and differentiating induced pluripotency cells (iPSCs) using machine learning.

### BACKGROUND

Induced pluripotent stem cells (iPSCs) are cells that have been reprogrammed into a pluripotent state. In the pluripotent state, cells can be differentiated for various therapeutic purposes. For example, motor neurons for treating neurological disorders (e.g., amyotrophic lateral sclerosis (ALS)) can be created from iPSCs. Developing the differentiated cells involves two processes: cell maintenance and cell differentiation. During cell maintenance, iPSCs are cultivated to have certain cellular characteristics (e.g., high quality, high confluence). After cell maintenance, the iPSCs may be subjected to one or more cell differentiation steps to obtain differentiated cells. These differentiated cells may be used to treat certain illnesses and/or test therapeutics.

The processes for maintaining and differentiating iPSCs to obtain differentiated cells (e.g., cells of a particular cell type) may include a number of decisions. Each of these decisions is, conventionally, performed or overseen by a human operator. For example, a human operator may visually inspect iPSCs to determine whether the iPSCs should be subjected to cell differentiation steps. However, human operators can introduce bias into the decision-making processes. For example, one human operator may classify a set of iPSCs as being “high-quality” while another human operator may classify the same set of iPSCs as being “medium-quality.” These inconsistencies can lead to non-uniform cell programming, which can impact the purity or specification of the differentiated cells for phenotypic analyses and/or therapeutic implementation.

Many of the decisions currently made by human operators involve those human operators reviewing images of iPSCs or portions of images of iPSCs. Due to the size and richness of these images, it can be time-consuming for a trained expert to fully analyze the iPSCs and determine what actions should be performed to the iPSCs (e.g., discard the cells, feed the cells, subject the cells to one or more cell differentiation processes, etc.). Additionally, as mentioned above, human operators can introduce bias into the decision-making process, further skewing the final product.

Therefore, solutions are needed to generate a self-supervised system for maintaining and differentiating iPSCs.

### BRIEF SUMMARY

Described are systems, methods, and programming for autonomously maintaining and differentiating iPSCs. The maintenance process may develop iPSCs that satisfy quality and confluence conditions. The differentiation process may subject these iPSCs to one or more cell differentiation steps. At a conclusion of the differentiation processes, differentiated cells may be obtained for phenotypic analyses and/or assays.

Traditionally, the maintenance and differentiation processes have been driven by human-operator decision making. At different points during the maintenance and differentiation processes, decisions need to be made as to identify what action is to be performed. For example, an individual, such as a trained expert, may analyze images of iPSCs to determine whether these iPSCs should be discarded, passaged into a new vessel, or have one or more reagents added to generate a particular type of differentiated cell. However, no standardized criteria for evaluating the iPSCs exists, and therefore the human-operators can introduce bias and/or inconsistencies into the decision-making process. Referring to the previous example, the individual may determine the quality of the iPSCs based on the expert's experience and training. Therefore, what one operator identifies as being “high quality” may be identified as “medium quality” by another operator. The actions performed to the iPSCs may therefore differ depending on the operator.

Human operators may visually inspect images depicting iPSCs to make their decisions. Therefore, machine learning models are well suited to replace these human-decision making steps. By automating these decision-making steps, biases introduced by the operator can be minimized. Furthermore, the evaluation process may take on prescribed standards regardless of the operators, which can also reduce variations and the amount of time needed to make the decisions. The result of the automation is the production of substantially uniform sets of high-quality, high confluence, differentiated cells that can be used for various phenotypic analyses.

In some embodiments, an autonomous system may be configured to select iPSCs for cell differentiation and subject the selected iPSCs to one or more cell differentiation steps to obtain differentiated cells. The selection of the iPSCs for cell differentiation may be a part of a maintenance process where iPSCs are cultivated for cell differentiation. During the maintenance process, an initial set of iPSCs may be retrieved from a cell storage system and thaw the retrieved iPSCs (e.g., the system may retrieve an initial set of iPSCs from a cell storage system and thaw the retrieved iPSCs). The iPSCs may be distributed across a plurality of sample containers on a slide plate. The slide plate may be a multi-well plate comprising a plurality of sample wells. Sample containers may also be referred to as “wells” or “cell cultures.” After being thawed, the system may feed the iPSCs stored in the sample containers. As used herein, “feed” or “feeding” refers to removing spent cell culture media and providing fresh cell culture media, of various compositions, to cells (e.g., iPSCs) in order to promote cell propagation and/or cell differentiation. In the maintenance process, the iPSCs are fed with cell culture media optimized for stem cell growth and maintenance of pluripotency (e.g., mTeSR™ Plus).

An imaging system (e.g., a digital microscopy imaging system) may be used to capture a plurality of images depicting the iPSCs stored in the sample containers. The images may be digital images. In some embodiments, the images may be brightfield, phase contrast, or fluorescence images. The system may provide the images depicting the iPSCs stored in the sample containers to one or more machine learning models to determine a quality score and/or confluence score for each sample container. The system may be configured to: discard iPSCs stored within a sample container if the quality score computed for the sample container is less than a first threshold quality score, feed iPSCs within a sample container having a quality score that is greater than the first threshold but less than or equal to a second threshold quality score, or select a sample container for passaging the iPSCs stored therein based on the quality score of the sample container being greater than the second threshold quality score. The system may also be configured to select one or more sample containers, where iPSCs stored in the selected sample containers may be passaged based on a confluence score for the iPSCs stored within the sample containers being within a threshold range of confluence scores. The iPSCs stored within sample containers having a confluence score outside the threshold range of confluence scores may be fed and imaged, and these images may be analyzed to determine whether the quality score and/or confluence score has improved.

The system may be configured to perform passaging of iPSCs distributed within the selected sample containers. Passaging refers to a process whereby cells, such as induced pluripotent cells, are harvested, transferred to culture vessels with fresh growth medium, and used to start new cultures. The system may subject the passaged iPSCs in the selected sample containers to one or more cell differentiation steps during the cell differentiation process.

During the cell differentiation process, the iPSCs in the selected sample containers may be fed and analyzed. The cells may be fed with different cell culture media in the cell differentiation process compared to the cell maintenance process in order to induce differentiation of the iPSCs. In the cell differentiation process, the iPSCs are fed with a cell specific cell culture media to induce differentiation into a particular cell type. Quality control checks may also be performed during the differentiation process to ensure that, the iPSCs specify correctly during the differentiation process. As a result of the differentiation process, the system may obtain differentiated cells. The differentiated cells may be stored and/or used for one or more phenotypic analyses, depending on the cell type that was produced. Exemplary analyses include, but are not limited to, assay evaluating cell viability, cell proliferation, and/or cytotoxicity.

In some embodiments, provided herein is a method for autonomous selection and maintenance of induced pluripotent stem cells (iPSCs), the method comprising: capturing a first plurality of images depicting a plurality of iPSCs stored within a first plurality of sample containers using an imaging system; determining, using one or more machine learning models, at least one of a quality score or a confluence score for each sample container of the first plurality of sample containers based on the first plurality of images, the quality score representing a quality of the iPSCs of the plurality of iPSCs distributed within each sample container and the confluence score representing a confluence of the iPSCs of the plurality of iPSCs distributed within each sample container; autonomously selecting at least one sample container from the first plurality of sample containers based on the at least one of the quality score or the confluence score of each of the first plurality of sample containers, wherein the at least one selected sample container comprises a subset of iPSCs of the plurality of iPSCs; and autonomously performing one or more maintenance operations on the subset of iPSCs stored within the at least one selected sample container.

In some embodiments, autonomously performing the one or more maintenance operations on the subset of iPSCs stored within the at least one selected sample container comprises: performing passaging on the subset of iPSCs stored within the at least one selected sample container, adding one or more reagents to the subset of iPSCs stored within the at least one selected sample container, banking the subset of iPSCs stored within the at least one selected sample container, performing a quality control (QC) check of the subset of iPSCs stored within the at least one selected sample container, performing a pluripotency status check of the subset of iPSCs stored within the at least one selected sample container, or discarding at least the subset of iPSCs stored within the at least one selected sample container.

In some embodiments, discarding at least the subset of iPSCs stored within the at least one selected sample container comprises: discarding the subset of iPSCs stored within the at least one selected sample container; or discarding the plurality of iPSCs stored within the first plurality of sample containers.

In some embodiments, autonomously performing the one or more maintenance operations on the subset of iPSCs stored within the at least one selected sample container comprises: performing passaging of the subset of iPSCs stored within the at least one selected sample container.

In some embodiments, the method further comprises: distributing, subsequent to the subset of iPSCs stored within the at least one selected sample container being passaged, the subset of iPSCs across a second plurality of sample containers; and optionally subjecting the distributed subset of iPSCs stored in the second plurality of sample containers to one or more cell differentiation steps.

In some embodiments, subjecting the distributed subset of iPSCs stored in each of the second plurality of sample containers to the one or more cell differentiation steps comprises: feeding the distributed subset of iPSCs stored in each of the second plurality of sample containers; and capturing a second plurality of images depicting the distributed subset of iPSCs stored in each of the second plurality of sample containers using the imaging system.

In some embodiments, the method further comprises: banking the distributed subset of iPSCs stored in the second plurality of sample containers based on a determination that a first predefined amount of time has elapsed from the feeding of the distributed subset of iPSCs stored in each of the second plurality of sample containers.

In some embodiments, the method further comprises: selecting a sample container from the second plurality of sample containers for performing a QC check.

In some embodiments, the sample container is randomly selected from the second plurality of sample containers.

In some embodiments, the method further comprises: feeding a subset of iPSCs stored within the selected sample container from the second plurality of sample containers; and capturing a third plurality of images depicting the iPSCs stored within the selected sample container from the second plurality of sample containers.

In some embodiments, the method further comprises: determining that a second predefined amount of time has not elapsed from the feeding of the iPSCs stored within the selected sample container; and repeating the feeding and image capturing of the iPSCs stored within the selected sample container until the second predefined amount of time has elapsed from the feeding of the iPSCs stored within the selected sample container.

In some embodiments, the method further comprises: determining that a second predefined amount of time has elapsed from the feeding of the iPSCs stored within the selected sample container; and performing the QC check to the iPSCs stored within the selected sample container to obtain a QC score.

In some embodiments, the method further comprises: discarding the distributed subset of iPSCs stored in the second plurality of sample containers based on a determination that the QC score is less than a threshold QC score.

In some embodiments, the method further comprises: subjecting the distributed subset of iPSCs remaining stored within the second plurality of sample containers excluding the iPSCs stored within the selected sample container to one or more additional cell differentiation steps to obtain a plurality of differentiated cells.

In some embodiments, the method further comprises: performing one or more phenotypic assessments using at least some of the plurality of differentiated cells.

In some embodiments, the passaging comprises: washing the subset of iPSCs stored within the at least one selected sample container, incubating the washed subset of iPSCs with a dissociation reagent, triturating the incubated subset of iPSCs after a media is added to the incubated subset of iPSCs, transferring the triturated subset of iPSCs to a sample container block, centrifuging the transferred subset of iPSCs in the sample container block to pellet the centrifuged subset of iPSCs, performing a buffering exchange to the pelleted subset of iPSCs by aspirating the pelleted subset of iPSCs, and suspending the aspirated subset of iPSCs into the media.

In some embodiments, the method further comprises: performing one or more feedings to the plurality of iPSCs stored within the first plurality of sample containers prior to the first plurality of images being captured.

In some embodiments, the plurality of iPSCs include: 1,000 or more iPSCs, 10,000 or more iPSCs, 100,000 or more iPSCs, or 1,000,000 or more iPSCs.

In some embodiments, the method further comprises autonomously removing the plurality of iPSCs from cell storage using a cell handling system; and thawing the plurality of iPSCs using a cell thawing system, wherein the first plurality of images is captured after the thawing.

In some embodiments, the at least one sample container is selected based on the quality score of the at least one sample container being greater than or equal to a first threshold quality score.

In some embodiments, the quality score comprises one of: a low-quality score, a medium-quality score, or a high-quality score.

In some embodiments, the first threshold quality score is the medium-quality score.

In some embodiments, the at least one sample container is selected based on the confluence score of the at least one sample container being within a predefined range of confluence scores.

In some embodiments, autonomously selecting the at least one sample container further comprises: ranking the first plurality of sample containers based on the at least one of the quality score or the confluence score of each of the first plurality of sample containers, wherein the at least one sample container is selected from the first plurality of sample containers based on the ranking.

In some embodiments, the method further comprises: discarding one or more sample containers from the first plurality of sample containers based on at least one of the quality score or the confluence score of the one or more sample containers.

In some embodiments, the one or more sample containers are discarded based on at least one of: the quality score of the one or more sample containers being less than a threshold quality score, or the confluence score of the one or more sample containers being outside of a predefined range of confluence scores.

In some embodiments, the method further comprises: identifying one or more sample containers, wherein the one or more identified sample containers have at least one of: a quality score that is (i) less than a first threshold quality score and (ii) greater than or equal to a second threshold quality score, or a confluence score outside of a predefined range of confluence scores.

In some embodiments, the method further comprises: feeding iPSCs stored within the one or more identified sample containers; capturing a second plurality of images depicting the iPSCs stored within the one or more identified sample containers using the imaging system; and determining at least one of an updated quality score or an updated confluence score for each of the one or more identified sample containers using the one or more machine learning models.

In some embodiments, the method further comprises: selecting at least one of the one or more identified sample containers based on the at least one of the updated quality score or the updated confluence score of the one or more sample containers.

In some embodiments, the at least one of the one or more identified sample containers is selected based on at least one of: the updated quality score of the at least one of the one or more identified sample containers being greater than or equal to the first threshold quality score, or the updated confluence score of the at least one of the one or more identified sample containers being within the predefined range of confluence scores.

In some embodiments, the predefined range of confluence scores is from about 20% to about 90%.

In some embodiments, the predefined range of confluence scores is from about 25% to about 85%.

In some embodiments, the predefined range of confluence scores is from about 30% to about 80%.

In some embodiments, the at least one sample container is selected based on the quality score of the at least one sample container being greater than or equal to a first threshold quality score and the confluence score of the at least one sample container being within a predefined range of confluence scores.

In some embodiments, the one or more machine learning models comprise a first machine learning model trained to determine a quality score representing a quality of the iPSCs stored within each of the first plurality of sample containers.

In some embodiments, the one or more machine learning models comprise a second machine learning model trained to determine a confluence score representing a confluence of the iPSCs stored within each of the first plurality of sample containers.

In some embodiments, the one or more machine learning models comprise a first machine learning model trained to determine a confluence score representing a quality of the iPSCs stored within each of the first plurality of sample containers.

In some embodiments, the one or more machine learning models comprise a second machine learning model trained to determine a quality score representing a quality of the iPSCs stored within each of the first plurality of sample containers.

In some embodiments, at least one of the first machine learning model or the second machine learning model comprise a convolutional neural network.

In some embodiments, the first machine learning model is built on a ResNet architecture and the second machine learning model is built on a U-Net architecture.

In some embodiments, the imaging system comprises a digital microscopy imaging system.

In some embodiments, the imaging system captures bright-field, phase contrast, or fluorescent images.

In some embodiments, the first plurality of sample containers is disposed on a slide plate.

In some embodiments, the slide plate is a multi-well plate comprising a plurality of sample wells.

In some embodiments, provided herein is a non-transitory computer-readable medium storing computer program instructions that, when executed by one or more processors, effectuates operations for autonomous selection and maintenance of induced pluripotent stem cells (iPSCs), the operations comprising: capturing a first plurality of images depicting a plurality of iPSCs stored within a first plurality of sample containers using an imaging system; determining, using one or more machine learning models, at least one of a quality score or a confluence score for each sample container of the first plurality of sample containers based on the first plurality of images, the quality score representing a quality of the iPSCs of the plurality of iPSCs distributed within each sample container and the confluence score representing a confluence of the iPSCs of the plurality of iPSCs distributed within each sample container; autonomously selecting at least one sample container from the first plurality of sample containers based on the at least one of the quality score or the confluence score of each of the first plurality of sample containers, wherein the at least one selected sample container comprises a subset of iPSCs of the plurality of iPSCs; and autonomously performing one or more maintenance operations on the subset of iPSCs stored within the at least one selected sample container.

In some embodiments, autonomously performing the one or more maintenance operations on the at least one selected sample container comprises: performing passaging on the subset of iPSCs stored within the at least one selected sample container, adding one or more reagents to the subset of iPSCs stored within the at least one selected sample container, banking the subset of iPSCs stored within the at least one selected sample container, performing a quality control check of the subset of iPSCs stored within the at least one selected sample container, performing a pluripotency status check of the subset of iPSCs stored within the at least one selected sample container, or discarding at least the subset of iPSCs stored within the at least one selected sample container.

In some embodiments, provided herein is a system for autonomous selection and maintenance of induced pluripotent stem cells (iPSCs), the system comprising: a computing system programmed to: capture a first plurality of images depicting a plurality of iPSCs stored within a first plurality of sample containers using an imaging system; determine, using one or more machine learning models, at least one of a quality score or a confluence score for each sample container of the first plurality of sample containers based on the first plurality of images, the quality score representing a quality of the iPSCs of the plurality of iPSCs distributed within each sample container and the confluence score representing a confluence of the iPSCs of the plurality of iPSCs distributed within each sample container; autonomously select at least one sample container from the first plurality of sample containers based on the at least one of the quality score or the confluence score of each of the first plurality of sample containers, wherein the at least one selected sample container comprises a subset of iPSCs of the plurality of iPSCs; and autonomously cause one or more maintenance operations to be performed on the subset of iPSCs stored within the at least one selected sample container.

In some embodiments, the one or more maintenance operations on the at least one selected sample container comprises: performing passaging on the subset of iPSCs stored within the at least one selected sample container, adding one or more reagents to the subset of iPSCs stored within the at least one selected sample container, banking the subset of iPSCs stored within the at least one selected sample container, performing a quality control check of the subset of iPSCs stored within the at least one selected sample container, performing a pluripotency status check of the subset of iPSCs stored within the at least one selected sample container, or discarding at least the subset of iPSCs stored within the at least one selected sample container.

In some embodiments, the system further comprises: the imaging system, wherein the imaging system is configured to capture the first plurality of images.

In some embodiments, the first plurality of images comprises bright-field, phase contrast, or fluorescent images.

In some embodiments, the imaging system comprises a digital microscopy imaging system.

In some embodiments, the computing system is further programmed to: output an instruction to cause the imaging system to capture the first plurality of images.

In some embodiments, the system further comprises: a passaging system configured to perform passaging of the subset of iPSCs stored within the at least one selected sample container.

In some embodiments, the system further comprises: a cell differentiation system configured to: distribute the subset of iPSCs across a second plurality of sample containers; and subject the iPSCs distributed within the second plurality of sample containers to one or more cell differentiation steps.

In some embodiments, the cell differentiation system is further configured to: feed the iPSCs distributed within the second plurality of sample containers.

In some embodiments, the computing system is further programmed to: instruct the imaging system to capture a second plurality of images depicting the iPSCs stored within each of the second plurality of sample containers.

In some embodiments, the computing system is further configured to: determine that a first predefined amount of time has elapsed from the feeding of the iPSCs stored within the second plurality of sample containers.

In some embodiments, the system further comprises: a cell storage system configured to store the second plurality of sample containers based on the determination that the first predefined amount of time has elapsed.

In some embodiments, the computing system is further configured to: select a sample container from the second plurality of sample containers to perform a quality control (QC).

In some embodiments, the cell differentiation system is further configured to: feed the iPSCs stored in the selected sample container.

In some embodiments, the computing system is further programmed to: instruct the imaging system to capture a third plurality of images depicting the iPSCs stored within the selected sample container.

In some embodiments, the computing system is further programmed to: determine that a second predefined amount of time has not elapsed from the feeding of the iPSCs stored within the selected sample container; and perform the QC check to the iPSCs stored within the selected sample container to obtain a QC score.

In some embodiments, the computing system is further programmed to: cause the iPSCs distributed within the second plurality of sample containers to be discarded based on the QC score being less than a threshold QC score.

In some embodiments, the cell differentiation system is further configured to: subject iPSCs remaining within the second plurality of sample containers excluding the selected sample container to one or more additional cell differentiation steps to obtain a plurality of differentiated cells.

In some embodiments, the computing system is further programmed to: cause one or more phenotypic assessments to be performed using at least some of the plurality of differentiated cells.

In some embodiments, passaging comprises: washing the subset of iPSCs stored within the at least one selected sample container, incubating the washed subset of iPSCs with a dissociation reagent, triturating the incubated subset of iPSCs after a media is added to the incubated subset of iPSCs, transferring the triturated subset of iPSCs to a sample container block, centrifuging the transferred subset of iPSCs in each sample container block to pellet the centrifuged subset of iPSCs, performing a buffering exchange to the pelleted subset of iPSCs by aspirating the pelleted subset of iPSCs, and suspending the aspirated subset of iPSCs into the media.

In some embodiments, the plurality of iPSCs include: 1,000 or more iPSCs, 10,000 or more iPSCs, 100,000 or more iPSCs, or 1,000,000 or more iPSCs.

In some embodiments, the system further comprises: a cell storage system configured to store the plurality of iPSCs; a cell thawing system configured to thaw iPSCs; and a cell handling system configured to transport iPSCs to at least one of the cell storage or the cell thawing system.

In some embodiments, the cell handling system is configured to: autonomously remove the plurality of iPSCs from cell storage; and autonomously provide the plurality of iPSCs stored within the first plurality of sample containers to the cell thawing system, the plurality of iPSCs being thawed by the cell thawing system.

In some embodiments, the cell handling system is further configured to: provide the thawed iPSCs stored within the first plurality of sample containers to the imaging system, wherein the first plurality of images is captured after the thawing.

In some embodiments, the at least one sample container is selected based on the quality score of the at least one sample container being greater than or equal to a first threshold quality score.

In some embodiments, the quality score comprises one of: a low-quality score, a medium-quality score, or a high-quality score.

In some embodiments, the first threshold quality score is the medium-quality score.

In some embodiments, the at least one sample container is selected based on the confluence score of the at least one sample container being within a predefined range of confluence scores.

In some embodiments, the computing system being programmed to select the at least one sample container comprises the computing system being programmed to: rank the first plurality of sample containers based on the at least one of the quality score or the confluence score of each of the first plurality of sample containers, wherein the at least one sample container is selected from the first plurality of sample containers based on the ranking.

In some embodiments, the system further comprises: discarding one or more sample containers from the first plurality of sample containers based on at least one of the quality score or the confluence score of the one or more sample containers.

In some embodiments, the computing system is further programmed to: identify one or more sample containers of the first plurality of sample containers, wherein the one or more identified sample containers have at least one of: a quality score that is (i) less than a first threshold quality score and (ii) greater than or equal to a second threshold quality score, or a confluence score outside of a predefined range of confluence scores.

In some embodiments, the system further comprises: a cell feeding system configured to feed iPSCs stored within the one or more identified sample containers.

In some embodiments, the system further comprises: the imaging system, wherein the imaging system is configured to: capture a second plurality of images depicting the iPSCs stored within the one or more identified sample containers.

In some embodiments, the computing system is further programmed to: determine at least one of an updated quality score or an updated confluence score for each of the one or more identified sample containers using the one or more machine learning models.

In some embodiments, the computing system is further programmed to: select at least one of the one or more identified sample containers based on the at least one of the updated quality score or the updated confluence score of the one or more sample containers.

In some embodiments, the at least one of the one or more identified sample containers is selected based on at least one of: the updated quality score of the at least one of the one or more identified sample containers being greater than or equal to the first threshold quality score, or the updated confluence score of the at least one of the one or more identified sample containers being within the predefined range of confluence scores.

In some embodiments, the predefined range of confluence scores is from about 20% to about 90%.

In some embodiments, the predefined range of confluence scores is from about 25% to about 85%.

In some embodiments, the predefined range of confluence scores is from about 30% to about 80%.

In some embodiments, the at least one sample container is selected based on the quality score of the at least one sample container being greater than or equal to a first threshold quality score and the confluence score of the at least one sample container being within a predefined range of confluence scores.

In some embodiments, the one or more machine learning models comprise a first machine learning model trained to determine a quality score representing a quality of the iPSCs stored within each of the first plurality of sample containers.

In some embodiments, the one or more machine learning models comprise a second machine learning model trained to determine a confluence score representing a confluence of the iPSCs stored within each of the first plurality of sample containers.

In some embodiments, at least one of the first machine learning model or the second machine learning model comprise a convolutional neural network.

In some embodiments, the first machine learning model is built on a ResNet architecture and the second machine learning model is built on a U-Net architecture.

In some embodiments, the first plurality of sample containers is disposed on a slide plate.

In some embodiments, the slide plate is a multi-well plate comprising a plurality of sample wells.

An exemplary method for autonomous selection and maintenance of induced pluripotent stem cells (iPSCs) comprises: capturing a first plurality of images depicting a plurality of iPSCs stored within a first plurality of sample containers using an imaging system; determining, using one or more machine learning models, a confluence score for each sample container of the first plurality of sample containers based on the first plurality of images, the confluence score representing a confluence of the iPSCs of the plurality of iPSCs distributed within each sample container; autonomously selecting at least one sample container from the first plurality of sample containers based on the confluence score of each of the first plurality of sample containers, wherein the at least one selected sample container comprises a subset of iPSCs of the plurality of iPSCs; and autonomously performing one or more maintenance operations on the subset of iPSCs stored within the at least one selected sample container.

In some embodiments, the method further comprises determining, using the one or more machine learning models, a quality score for each sample container of the first plurality of sample containers based on the first plurality of images, the quality score representing a quality of the iPSCs of the plurality of iPSCs distributed within each sample container, wherein autonomously selecting the at least one sample container comprises: autonomously selecting the at least one sample container from the first plurality of sample containers based on the confluence score and the quality score of each of the first plurality of sample containers.

In some embodiments, the quality score comprises a binary value, a numeric value, a classification, or any combination thereof.

In some embodiments, the quality score comprises one of: a low-quality score, a medium-quality score, or a high-quality score.

In some embodiments, the method further comprises determining a growth metric for each sample container of the first plurality of sample containers based on the first plurality of images, the growth metric representing a growth status of the iPSCs of the plurality of iPSCs distributed within each sample container, wherein autonomously selecting the at least one sample container comprises: autonomously selecting the at least one sample container from the first plurality of sample containers based on the confluence score, the quality score, and the growth metric of each of the first plurality of sample containers.

In some embodiments, the growth metric is indicative of whether the iPSCs of the plurality of iPSCs distributed within each sample container is in a growth phase.

In some embodiments, the growth metric is indicative of a growth rate of the iPSCs of the plurality of iPSCs distributed within each sample container.

In some embodiments, the growth metric is indicative of whether the growth rate of the iPSCs of the plurality of iPSCs distributed within each sample container is positive.

In some embodiments, the growth metric is determined based on a first confluence score and a second confluence score of the plurality of iPSCs distributed within each sample container, wherein the first confluence score is associated with a first time point, and wherein the second confluence score is associated with a second time point later than the first time point.

In some embodiments, the growth metric indicates positive growth if the second confluence score is higher than the first confluence score.

In some embodiments, autonomously selecting the at least one sample container from the first plurality of sample containers comprises: obtaining a ranking of a plurality of predefined confluence score ranges, wherein the plurality of predefined confluence score ranges comprises a first predefined confluence score range ranked higher than a second predefined confluence score range; and prioritizing selection of a sample having a confluence score in the first predefined confluence score range over a sample having a confluence score in the second predefined confluence score range.

In some embodiments, autonomously selecting the at least one sample container from the first plurality of sample containers comprises: prioritizing selection of a sample having a higher quality score over a sample having a lower quality score.

In some embodiments, autonomously selecting the at least one sample container from the first plurality of sample containers comprises: prioritizing selection of a sample having a higher or positive growth metric over a sample having a lower or negative growth metric.

In some embodiments, autonomously selecting the at least one sample container from the first plurality of sample containers comprises: prioritizing selection of a sample having a confluence score in the second predefined confluence score range and a higher quality score over a sample having a confluence score in the first predefined confluence score range and a lower quality score.

In some embodiments, autonomously performing the one or more maintenance operations on the subset of iPSCs stored within the at least one selected sample container comprises: performing passaging on the subset of iPSCs stored within the at least one selected sample container, adding one or more reagents to the subset of iPSCs stored within the at least one selected sample container, banking the subset of iPSCs stored within the at least one selected sample container, performing a quality control (QC) check of the subset of iPSCs stored within the at least one selected sample container, performing a pluripotency status check of the subset of iPSCs stored within the at least one selected sample container, or discarding at least the subset of iPSCs stored within the at least one selected sample container.

In some embodiments, discarding at least the subset of iPSCs stored within the at least one selected sample container comprises: discarding the subset of iPSCs stored within the at least one selected sample container; or discarding the plurality of iPSCs stored within the first plurality of sample containers.

In some embodiments, autonomously performing the one or more maintenance operations on the subset of iPSCs stored within the at least one selected sample container comprises: performing passaging of the subset of iPSCs stored within the at least one selected sample container.

In some embodiments, the method further comprises distributing, subsequent to the subset of iPSCs stored within the at least one selected sample container being passaged, the subset of iPSCs across a second plurality of sample containers; and optionally subjecting the distributed subset of iPSCs stored in the second plurality of sample containers to one or more cell differentiation steps.

In some embodiments, subjecting the distributed subset of iPSCs stored in each of the second plurality of sample containers to the one or more cell differentiation steps comprises: feeding the distributed subset of iPSCs stored in each of the second plurality of sample containers; and capturing a second plurality of images depicting the distributed subset of iPSCs stored in each of the second plurality of sample containers using the imaging system.

In some embodiments, the method further comprises banking the distributed subset of iPSCs stored in the second plurality of sample containers based on a determination that a first predefined amount of time has elapsed from the feeding of the distributed subset of iPSCs stored in each of the second plurality of sample containers.

In some embodiments, the method further comprises selecting a sample container from the second plurality of sample containers for performing a QC check.

In some embodiments, the sample container is randomly selected from the second plurality of sample containers.

In some embodiments, the method further comprises feeding a subset of iPSCs stored within the selected sample container from the second plurality of sample containers; and capturing a third plurality of images depicting the iPSCs stored within the selected sample container from the second plurality of sample containers.

In some embodiments, the method further comprises determining that a second predefined amount of time has not elapsed from the feeding of the iPSCs stored within the selected sample container; and repeating the feeding and image capturing of the iPSCs stored within the selected sample container until the second predefined amount of time has elapsed from the feeding of the iPSCs stored within the selected sample container.

In some embodiments, the method further comprises determining that a second predefined amount of time has elapsed from the feeding of the iPSCs stored within the selected sample container; and performing the QC check to the iPSCs stored within the selected sample container to obtain a QC score.

In some embodiments, the method further comprises discarding the distributed subset of iPSCs stored in the second plurality of sample containers based on a determination that the QC score is less than a threshold QC score.

In some embodiments, the method further comprises subjecting the distributed subset of iPSCs remaining stored within the second plurality of sample containers excluding the iPSCs stored within the selected sample container to one or more additional cell differentiation steps to obtain a plurality of differentiated cells.

In some embodiments, the method further comprises performing one or more phenotypic assessments using at least some of the plurality of differentiated cells.

In some embodiments, passaging comprises: washing the subset of iPSCs stored within the at least one selected sample container, incubating the washed subset of iPSCs with a dissociation reagent, triturating the incubated subset of iPSCs after a media is added to the incubated subset of iPSCs, transferring the triturated subset of iPSCs to a sample container block, centrifuging the transferred subset of iPSCs in the sample container block to pellet the centrifuged subset of iPSCs, performing a buffering exchange to the pelleted subset of iPSCs by aspirating the pelleted subset of iPSCs, and suspending the aspirated subset of iPSCs into the media.

In some embodiments, the method further comprises performing one or more feedings to the plurality of iPSCs stored within the first plurality of sample containers prior to the first plurality of images being captured.

In some embodiments, the plurality of iPSCs include: 1,000 or more iPSCs, 10,000 or more iPSCs, 100,000 or more iPSCs, or 1,000,000 or more iPSCs.

In some embodiments, the method further comprises autonomously removing the plurality of iPSCs from cell storage using a cell handling system; and thawing the plurality of iPSCs using a cell thawing system, wherein the first plurality of images is captured after the thawing.

In some embodiments, the at least one sample container is selected based on the quality score of the at least one sample container being greater than or equal to a first threshold quality score.

In some embodiments, the first threshold quality score is the medium-quality score.

In some embodiments, the at least one sample container is selected based on the confluence score of the at least one sample container being within a predefined range of confluence scores.

In some embodiments, autonomously selecting the at least one sample container further comprises: ranking the first plurality of sample containers based on the at least one of the quality score or the confluence score of each of the first plurality of sample containers, wherein the at least one sample container is selected from the first plurality of sample containers based on the ranking.

In some embodiments, the method further comprises discarding one or more sample containers from the first plurality of sample containers based on at least one of the quality score, the confluence score, and/or the growth metric of the one or more sample containers.

In some embodiments, the one or more sample containers are discarded based on at least one of: the quality score of the one or more sample containers being less than a threshold quality score, the confluence score of the one or more sample containers being outside of a predefined range of confluence scores, and/or the growth metric of the one or more sample containers indicating a growth rate that is negative or being less than a growth metric threshold.

In some embodiments, the method further comprises identifying one or more sample containers, wherein the one or more identified sample containers have at least one of: a quality score that is (i) less than a first threshold quality score and (ii) greater than or equal to a second threshold quality score, a confluence score outside of a predefined range of confluence scores, or a growth metric indicating a growth rate that is negative or being less than a growth metric threshold.

In some embodiments, the method further comprises feeding iPSCs stored within the one or more identified sample containers; capturing a second plurality of images depicting the iPSCs stored within the one or more identified sample containers using the imaging system; and determining at least one of an updated quality score, an updated confluence score, or an updated growth metric for each of the one or more identified sample containers using the one or more machine learning models.

In some embodiments, the method further comprises selecting at least one of the one or more identified sample containers based on the at least one of the updated quality score, the updated confluence score, or the updated growth metric of the one or more sample containers.

In some embodiments, the at least one of the one or more identified sample containers is selected based on at least one of: the updated quality score of the at least one of the one or more identified sample containers being greater than or equal to the first threshold quality score, the updated confluence score of the at least one of the one or more identified sample containers being within the predefined range of confluence scores, or the updated growth metric of the least one of the one or more identified sample containers indicating a growth rate that is positive or being more than the growth metric threshold.

In some embodiments, the predefined range of confluence scores is from about 20% to about 90%.

In some embodiments, the predefined range of confluence scores is from about 25% to about 85%.

In some embodiments, the predefined range of confluence scores is from about 30% to about 80%.

In some embodiments, the at least one sample container is selected based on the quality score of the at least one sample container being greater than or equal to a first threshold quality score, the confluence score of the at least one sample container being within a predefined range of confluence scores, and the growth metric indicating a growth rate that is positive or being more than the growth metric threshold.

In some embodiments, the one or more machine learning models comprise a first machine learning model trained to determine a quality score representing a quality of the iPSCs stored within each of the first plurality of sample containers.

In some embodiments, the one or more machine learning models comprise a second machine learning model trained to determine a confluence score representing a confluence of the iPSCs stored within each of the first plurality of sample containers.

In some embodiments, at least one of the first machine learning model or the second machine learning model comprise a convolutional neural network.

In some embodiments, the first machine learning model is built on a ResNet architecture and the second machine learning model is built on a U-Net architecture.

In some embodiments, the imaging system comprises a digital microscopy imaging system.

In some embodiments, the imaging system captures bright-field, phase contrast, or fluorescent images.

In some embodiments, the first plurality of sample containers is disposed on a slide plate.

In some embodiments, the slide plate is a multi-well plate comprising a plurality of sample wells. An exemplary system comprises: one or more processors; a memory; and one or more programs, wherein the one or more programs are stored in the memory and configured to be executed by the one or more processors, the one or more programs including instructions for performing any of the methods described herein.

An exemplary non-transitory computer-readable storage medium stores one or more programs, the one or more programs comprising instructions, which when executed by one or more processors of an electronic device, cause the electronic device to perform any of the methods described herein.

### DESCRIPTION OF THE FIGURES

FIG. 1A illustrates a system including an autonomous maintenance and differentiation platform, in accordance with some embodiments.

FIG. 1B illustrates a computing system forming a component of the autonomous maintenance and differentiation platform of FIG. 1A, in accordance with some embodiments.

FIG. 1C illustrates an exemplary sample ranking scheme, in accordance with some embodiments.

FIG. 2 illustrates an exemplary method for autonomously maintaining and differentiating iPSCs to obtain differentiated cells for phenotyping, in accordance with some embodiments.

FIG. 3 illustrates an example maintenance processing system for performing maintenance to iPSCs, in accordance with some embodiments.

FIG. 4A illustrates an exemplary method for selecting, based on a maintenance process performed to iPSCs, at least one sample container of iPSCs for passaging, in accordance with some embodiments.

FIG. 4B illustrates an exemplary method for performing passaging of iPSCs stored within one or more selected sample containers, in accordance with some embodiments.

FIG. 5 illustrates an example differentiation processing system for performing cell differentiation steps, in accordance with some embodiments.

FIGS. 6A-6D illustrate an exemplary method for subjecting iPSCs to one or more cell differentiation steps, in accordance with some embodiments.

FIG. 7 illustrates an example method for training one or more machine learning models to determine a quality score of sample containers storing iPSCs, in accordance with various embodiments.

FIG. 8 illustrates an example method for training one or more machine learning models to determine a confluence score of sample containers storing iPSCs, in accordance with some embodiments.

FIGS. 9A-9C illustrate example bright-field image and corresponding mask representations, in accordance with some embodiments.

FIGS. 10A-10B illustrate example images of sample containers storing iPSCs having different confluence scores, in accordance with some embodiments.

FIG. 11 illustrates an example image of a sample container storing iPSCs presented to an individual, such as a trained pathologist, via a web interface, in accordance with some embodiments.

FIGS. 12A-12D illustrate example bright-field images of sample containers storing iPSCs, and mask representations generated based on the bright-field images, in accordance with some embodiments.

FIG. 13 illustrates an example growth curve depicting a change in confluence of a sample container over time, in accordance with some embodiments.

FIG. 14 illustrates an example sample plate map including information about a plurality of sample containers, in accordance with some embodiments.

FIG. 15 illustrates an exemplary computing system used to implement one or more embodiments described herein.

FIG. 16 illustrates an example bright-field image of sample containers classified as storing “low-quality” iPSCs, in accordance with some embodiments.

FIG. 17 illustrates an example bright-field image of sample containers classified as storing “medium-quality” iPSCs, in accordance with some embodiments.

FIG. 18 illustrates an example bright-field image of sample containers classified as storing “high-quality” iPSCs, in accordance with some embodiments.

FIG. 19 illustrates example bright-field images of sample containers classified as storing “low-quality” iPSCs, in accordance with some embodiments.

FIG. 20 illustrates example bright-field images of sample containers classified as storing “medium-quality” iPSCs, in accordance with some embodiments.

FIG. 21 illustrates example bright-field images of sample containers classified as storing “high-quality” iPSCs, in accordance with some embodiments.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown but are to be accorded the scope consistent with the claims.

Disclosed herein are methods, systems, electronic devices, non-transitory storage media, and apparatuses directed to an autonomous cell maintenance and cell differentiation platform. The autonomous cell maintenance and cell differentiation platform may be used to autonomously cultivate differentiated cells. These differentiated cells can be used for assays, phenotypic analyses, and/or other therapeutic purposes. In some embodiments, the autonomous cell maintenance and cell differentiation platform may implement machine learning models to evaluate a quality and a confluence of sample containers storing iPSCs. Based on the quality and the confluence, at least one of the sample containers may be selected. A subset of iPSCs stored within the selected sample container(s) may be passaged and subjected to one or more cell differentiation steps to obtain the differentiated cells.

It should be noted that, although some embodiments are described herein with respect to machine learning models, other prediction models (e.g., statistical models or other analytics models) may be used in lieu of or in addition to machine learning models in other embodiments (e.g., a statistical model replacing a machine-learning model and a non-statistical model replacing a non-machine-learning model in one or more embodiments).

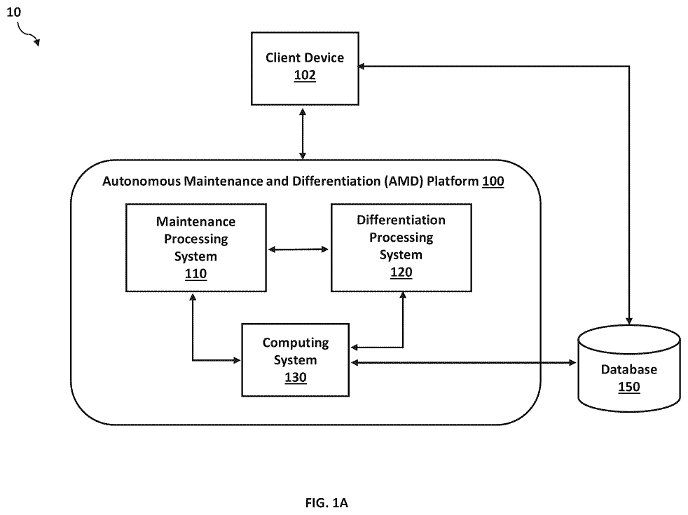

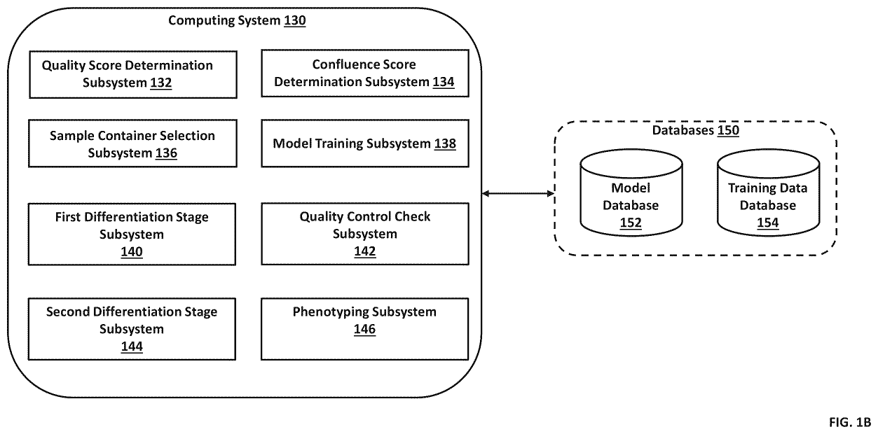

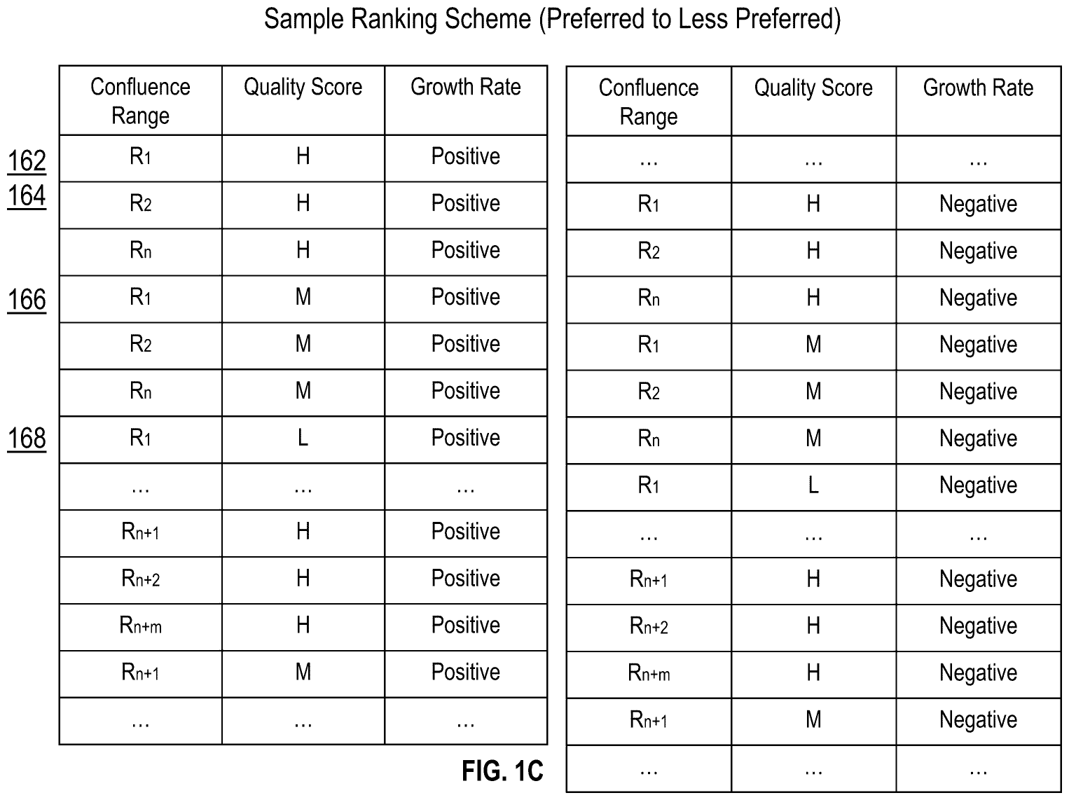

FIG. 1A illustrates a system 10 including an autonomous maintenance and differentiation (AMD) platform 100, in accordance with some embodiments. AMD platform 100 may be configured to communicate with one or more databases 150, one or more client devices 102, or other components of system 10 using one or more networks (e.g., the Internet). Although FIG. 1A illustrates system 10 including a single instance of database 150 and client device 102, persons of ordinary skill in the art will recognize that system 10 may include additional databases, additional client devices, or other components. For example, AMD platform 100, client device 102, and databases 150 may communicate with an imaging system configured to capture digital images.

AMD platform 100 may include a maintenance processing system 110, a differentiation processing system 120, a computing system 130, or other components. Each of maintenance processing system 110, differentiation processing system 120, and computing system 130 may communicate with one another, databases 150, and/or client device 102 using one or more networks (e.g., the Internet, an Intranet). Maintenance processing system 110 may include systems and devices for developing iPSCs having similar characteristics (e.g., high quality, high confluence). These iPSCs may then be subjected to one or more cell differentiation steps using differentiation processing system 120 to obtain differentiated cells. The differentiated cells may be used for one or more assays, phenotypic analyses, therapeutic interventions, or for other purposes.

In some embodiments, AMD platform 100 may perform cell maintenance to iPSCs using maintenance processing system 110. Maintenance processing system 110 may be configured to perform various cell maintenance steps to prepare iPSCs for cell differentiation. The cell maintenance steps may include, for example, retrieving iPSCs from cell storage, distributing the iPSCs across a plurality of sample containers (e.g., a plurality of sample wells), thawing the iPSCs, feeding the iPSCs, imaging the iPSCs (e.g., via an imaging system), and selecting one or more of the sample containers—each storing a subset of the iPSCs—for cell differentiation.

In some embodiments, maintenance processing system 110 may select one or more of the sample containers for cell differentiation based on a quality score of each sample container. The quality score represents a quality of the subset of iPSCs stored within each sample container. The quality score may be a binary value, a continuous score (e.g., a numerical value between 0 and 100), or classified score (e.g., “low,” “medium,” “high,” “empty”). For example, the quality score can be one of: high quality, medium quality, low quality. As another example, the quality score can be a numerical value on a scale of 1 to 10, 1 to 100, 1 to 1000, or the like. As another example, the quality score can be a relative value such as a percentile value (e.g., top 1 percentile, top 2 percentile, or the like). As another example, the quality score can be a numerical value such as a percentage. As another example, the quality score can be a grade such as A, B, C, D, or the like. Further, each numerical score can be translated into a classified score. For example, for numerical scores on the scale of 1-100, a numerical score above a first threshold (e.g., 67-100) can be translated into a classification of high quality; a numerical score below the first threshold and above a second threshold (e.g., 34-66) can be translated into a classification of medium quality; a numerical score below the second threshold (e.g., 0-33) can be translated into a classification of low quality. One of ordinary skill should appreciate that the quality score can be of any value indicative of absolute or relative quality of a sample. In some embodiments, maintenance processing system 110 may implement one or more machine learning models to determine the quality scores. For example, a convolutional neural network (CNN) may analyze images of the sample containers and output a quality score.

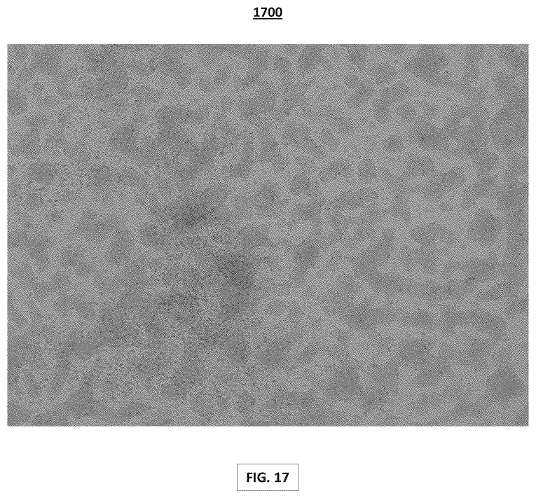

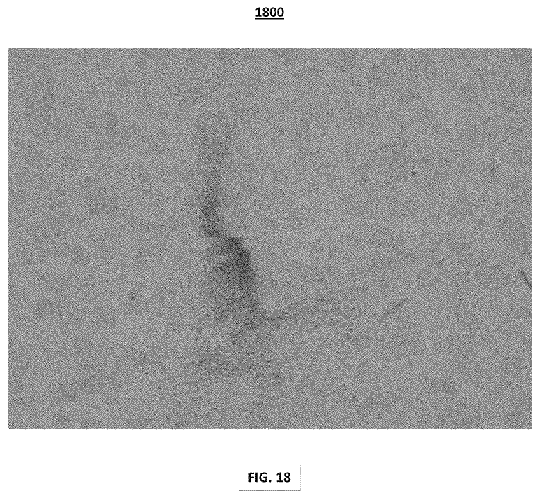

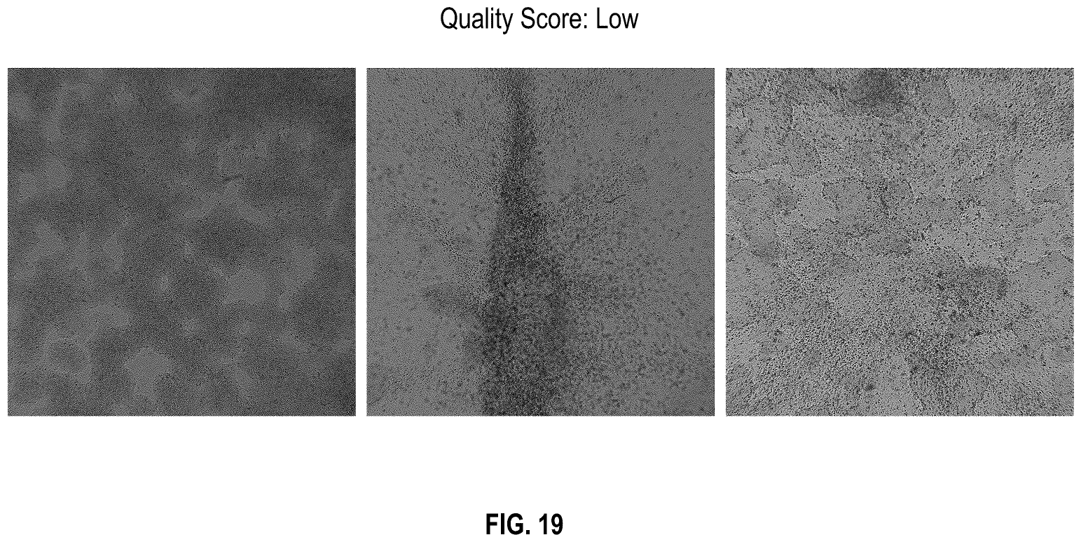

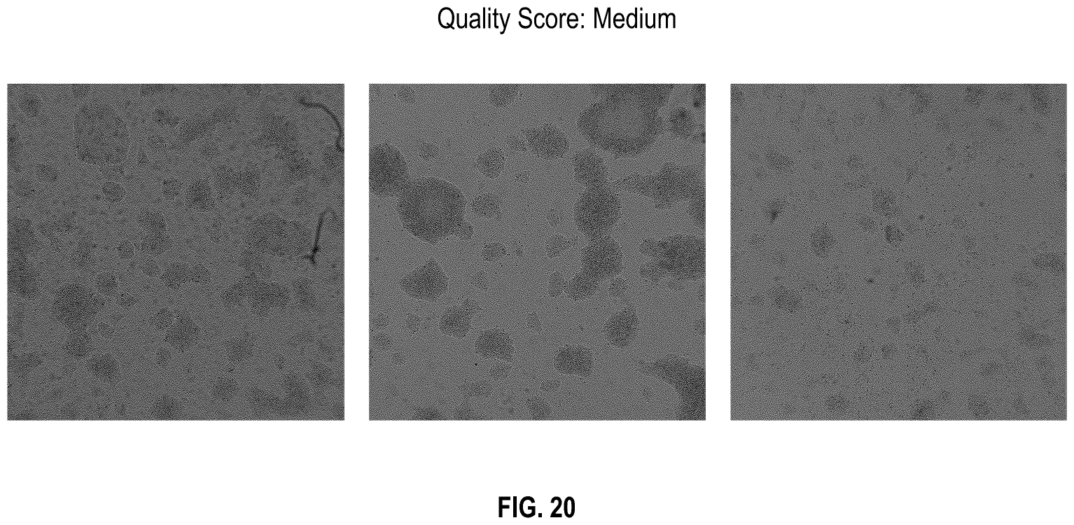

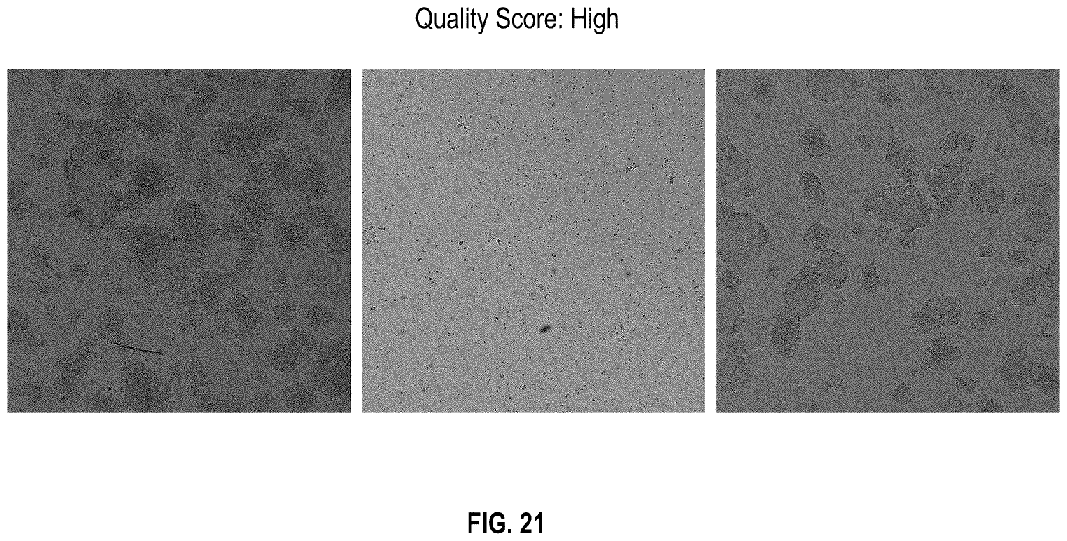

Maintenance processing system 110 may determine which, if any, actions to perform to the iPSCs stored within each sample container based on the quality scores. For example, sample containers classified as storing “low-quality” iPSCs may be discarded, sample containers classified as storing “medium-quality” iPSCs may be subjected to one or more feedings, and sample containers classified as storing “high-quality” iPSCs may be selected for passaging. An exemplary bright-field image of sample containers classified as storing “low-quality” iPSCs is shown in 1600, an exemplary bright-field image of sample containers classified as storing “medium-quality” iPSCs is shown in 1700, and an exemplary bright-field image of sample containers classified as storing “high-quality” iPSCs are shown in 1800 (FIGS. 16-18). FIGS. 20-22 illustrate additional example images of “low-quality”, “medium-quality”, and “high-quality” iPSCs. Sample containers classified as “empty” may store less than a threshold quantity of iPSCs and may be discarded. In some embodiments, maintenance processing system 110 may select one or more sample containers. The iPSCs stored in the selected sample containers may be subjected to one or more cell differentiation steps. For example, maintenance processing system 110 may select one or more of the sample containers based on those sample containers satisfying a quality score threshold condition. For example, a sample container satisfying the quality score threshold condition may include a quality score of that sample container being greater than or equal to a threshold quality score.

In some embodiments, prior to subjecting the iPSCs to cell differentiation steps, a confluence score may be computed for each sample container. The confluence score indicates how occupied a given sample container is with iPSCs. For example, highly populated sample containers may have a higher confluence score than less populated sample containers. The confluence score may be a continuous score (e.g., a numerical value between 0 and 100, a percentage) or classified score (e.g., “low,” “medium,” “high,” “empty”). In some embodiments, maintenance processing system 110 may implement one or more machine learning models to determine the confluence scores. For example, a CNN may analyze images of the sample containers and output a confluence score for each sample container. Maintenance processing system 110 may determine what actions to perform, if any, to the iPSCs stored within each sample container based on the confluence scores. For example, maintenance processing system 110 may select one or more of the sample containers based on those sample containers satisfying a confluence score threshold condition. For example, a sample container satisfying the confluence score threshold condition may include a confluence score of that sample container being within a predefined range of confluence scores. The iPSCs stored within those sample containers may be passaged. As another example, iPSCs stored within sample containers having a confluence score outside the predefined range of confluence scores may be subjected to one or more feedings.

In some embodiments, prior to subjecting the iPSCs to cell differentiation steps, a growth metric may be computed for each sample container. The growth metric for each sample represents a growth status of the iPSCs distributed within the corresponding sample container. The growth metric can be a binary value, a numerical value, or a classification. In some embodiments, the growth metric is indicative of whether the iPSCs of the plurality of iPSCs distributed within each sample container is in a growth phase. For example, the growth metric can be one of two classifications: in growth phrase and not in growth phase. As another example, the growth metric can be one of two classifications: positive (i.e., in growth phase) and negative (i.e., not in growth phase). As another example, the growth metric can be a numerical value indicative of a growth rate. If the numerical value exceeds a threshold (e.g., 0), the growth metric indicates positive growth. As discussed herein, the system can rank a sample having a higher or positive growth metric higher than a sample having a lower or negative growth metric.

In some embodiments, maintenance processing system 110 may select one or more sample containers based on the quality score, the confluence score, and/or the growth metric. In some embodiments, maintenance processing system 110 may select one or more of the sample containers based on those sample containers satisfying the quality score threshold condition, the confluence score threshold condition, and the growth metric threshold. For example, a sample container satisfying the quality score threshold condition may include a quality score of that sample container being greater than or equal to a threshold quality score. As another example, a sample container satisfying the confluence score threshold condition may have a confluence score of that sample container being within a predefined range of confluence scores. As another example, a sample container satisfying the growth metric threshold may have a growth metric of that sample container indicative of positive growth.

In some embodiments, machine learning models (e.g., a first machine learning model and a second machine learning model) may be trained to determine a quality score, a confluence score and/or a growth metric for each of the sample containers based on captured images of the sample containers. The machine learning models may further be trained to select one or more sample containers based on their quality score, the confluence score, and the growth metric. Using the machine learning models can increase the consistency of iPSCs selected for passaging and cell differentiation, which further improves quality and uniformity of the differentiated cells produced downstream. Thus, by selecting sample containers satisfying the quality score threshold condition, the confluence score threshold condition, and the growth metric threshold (e.g., the quality score being greater than or equal to the threshold quality score, a confluence score being within the predefined range of confluence scores, the growth metric indicative of positive growth), maintenance processing system 110 can produce more high-quality and consistent differentiated cells while also minimizing biases that can be introduced when relying on human evaluation techniques.

Maintenance processing system 110 may perform passaging of subsets of iPSCs stored within the selected sample containers. Passaging refers to a procedure for harvesting iPSCs from a culture, transferring the iPSCs to one or more culture vessels with a fresh growth medium, and using those iPSCs to start new cultures. Passaging may ensure that the iPSCs within a sample container maintain a particular level of quality and confluence so that those iPSCs may be subjected to cell differentiation. Passaging of the iPSCs may occur after a predefined amount of time has elapsed since thawing. In some embodiments, multiple rounds of passaging may be performed before subjecting the cells to one or more cell differentiation steps. For example, two rounds of passaging may be performed on the iPSCs stored within the selected sample containers prior to cell differentiation. In some embodiments, the cell differentiation steps may be optional.

In some embodiments, AMD platform 100 may subject the iPSCs to one or more differentiation steps using differentiation processing system 120. The goal of the cell differentiation steps is to develop cells of a particular type, such as motor neurons, retinal epithelial cells, epidermal cells, pancreatic β-islet cells, skeletal muscle cells, etc. Differentiation processing system 120 may subject the iPSCs to one or more feedings (e.g., exposing the iPSCs to key reprogramming factors). Different feeding materials may be used to produce different types of cells. In some embodiments, differentiation processing system 120 may use one or more machine learning models, statistical models, or other algorithms to determine when to subject cells to the differentiation steps. Furthermore, differentiation processing system 120 may perform quality checks and/or store differentiated cells for use in one or more assays, phenotypic analyses, and/or therapeutic interventions.

FIG. 1B illustrates a computing system 130 forming a component of AMD platform 100 of FIG. 1A, in accordance with some embodiments. Computing system 130 may include a quality score determination subsystem 132, a confluence score determination subsystem 134, a sample container selection subsystem 136, a model training subsystem 138, a first differentiation stage subsystem 140, a quality control (QC) check subsystem 142, a second differentiation stage subsystem 144, a phenotyping subsystem 146, or other components. Each of subsystems 132-146 may include one or more processors, memory, and communication components for performing certain operations. It should be noted that, while one or more operations are described herein as being performed by one of subsystems 132-146, those operations may, in some embodiments, be performed by other subsystems of computing system 130 or other components of system 10.

Quality score determination subsystem 132 may be configured to determine a quality score for each sample container based on images depicting the sample containers (e.g., sample wells). The quality score represents a quality of the iPSCs stored within a given sample container. In some embodiments, the sample container may be a sample plate. In some embodiments, the sample container may be a multi-well plate and can include a plurality of sample wells (e.g., 4 or more sample wells, 12 or more sample wells, etc.). A plurality of iPSCs may be retrieved from cell storage and distributed amongst the sample containers on the multi-well plate. Each sample container may store a subset of the iPSCs. The subsets of iPSCs may be fed one or more times with a growth medium. After being fed, the multi-well plate may be imaged using an imaging system. In some embodiments, the imaging system may capture a plurality of images of the multi-well plate. The images may be digital images. In some embodiments, the images may be bright-field, phase contrast, or fluorescent images. In some embodiments, the imaging system may capture images of portions of the multi-well plate. For example, one or more images may be captured of a subset of sample wells (e.g., an image depicting 4 sample wells). The number of images captured and the resolution of the images may vary depending on a type of imaging device used. For example, the captured images may have a resolution of 1,224×904 pixels. In some embodiments, the images may be digital images.

In some embodiments, quality score determination subsystem 132 may employ one or more machine learning models stored in model database 152 to determine a quality score based on the images of iPSCs stored within sample containers. For example, a trained machine learning model (e.g., a convolutional neural network (CNN)) may analyze the images and determine a quality score for each sample container. The quality score represents a quality of the subset of iPSCs stored within a given sample container. The quality can be a proxy for an overall health of the iPSCs stored within the sample container. Healthy iPSCs may be identified based on visual characteristics, such as, for example, well-defined cellular borders. Images depicting iPSCs with certain visual characteristics may indicate that those iPSCs are of a particular health level, which may be represented by the quality score. As an example, healthy iPSCs may have well-defined borders may be high quality, while iPSCs of poor health may have borders that are not well defined.

In some embodiments, quality score determination subsystem 132 may classify each sample container into one of a set of quality classifications based on the quality score determined by the machine learning models. For example, the machine learning models may generate a quality score for a given sample container. Quality score determination subsystem 132 may classify, using the one or more machine learning models, the quality score into one of a set of a quality classification. The set of quality classifications may include:
(i) an empty classification indicating that the sample container is empty of iPSCs (e.g., less than a threshold number of iPSCs are present in the sample container),
(ii) a low-quality classification indicating that the machine learning models computed that the subset of iPSCs stored within the sample container has a low-quality score,
(iii) a medium-quality classification indicating that the machine learning models computed that the subset of iPSCs stored within the sample container has a medium-quality score, and
(iv) a high-quality classification indicating that the machine learning models computed that the subset of iPSCs stored within the sample container has a high-quality score.

In some embodiments, the low-quality score refers to a quality score that is less than a first quality threshold score. In some embodiments, the medium-quality score refers to a quality score that is greater than or equal to the first quality threshold and less than a second quality threshold score. In some embodiments, the high-quality score refers to a quality score that is greater than or equal to the second quality threshold score.

In some embodiments, the machine learning models may determine a quality score for iPSCs stored within a sample container based on an embedding generated for the image depicting the sample container. The machine learning models may receive images depicting sample containers storing subsets of iPSCs and may generate an embedding for each image in a corresponding feature embedding space. As will be described in greater detail below, the image may be tiled (overlapping tiles or non-overlapping tiles), and an embedding may be produced for each tile. The embedding can be represented as a feature vector for the image. Quality score determination subsystem 132 can use a neural network (e.g., a convolutional neural network) to generate a feature vector that represents each image. In some embodiments, the embedding neural network can be based on a ResNet image network trained on a dataset based on natural (e.g., non-medical) images, such as the ImageNet dataset. By using a non-specialized tile embedding network, quality score determination subsystem 132 can leverage known advances in efficiently processing images to generate embeddings. Furthermore, using a natural image dataset allows the embedding neural network to learn to discern differences between segments of an image or a tile on a holistic level.

In some embodiments, the embedding network can be customized to handle large numbers of tiles of large format images, such as digital images. Additionally, the embedding network can be trained using a custom dataset. For example, the embedding network can be trained using a variety of samples of whole slide images or even trained using samples relevant to the subject matter for which the embedding network will be generating embeddings (e.g., scans of particular tissue types or actual tissue). Training the embedding network using specialized or customized sets of images can allow the embedding network to identify finer differences, which can result in more detailed and accurate distances between images in the feature embedding space at the cost of additional time to acquire the images and the computational and economic cost of training multiple embedding networks. Quality score determination subsystem 132 can select an embedding network from a library of embedding networks based on the type of images to be processed.

As described herein, embeddings can be generated from a deep learning neural network using visual features of the image and/or tiles of images. Embeddings can be further generated from contextual information associated with the images (e.g., content shown in the image). For example, an embedding can include one or more features that indicate and/or correspond to a size of depicted objects (e.g., sizes of depicted cells or aberrations) and/or density of depicted objects (e.g., a density of depicted cells or aberrations). Size and density can be measured absolutely (e.g., width expressed in pixels or converted from pixels to nanometers) or relative to tiles from the same digital image, from a class of digital images (e.g., produced using similar techniques or by a single imaging system or scanner), or from a related family of digital images. Furthermore, images can be classified prior to the embeddings being generated such that the classification are used during the preparation of the embeddings.

The embeddings may be of a predefined size (e.g., vectors of 512 elements, vectors of 2048 bytes, etc.). In some embodiments, embeddings of various and/or arbitrary sizes may be produced. The embeddings may also be adjusted based on user direction or can be selected, for example, to optimize computational efficiency, accuracy, or other parameters. In some embodiments, the embedding size can be based on the limitations or specifications of the deep learning neural network that generated the embeddings. Larger embedding sizes can be used to increase the amount of information captured in the embedding and improve the quality and accuracy of results, while smaller embedding sizes can be used to improve computational efficiency.

In some embodiments, quality score determination subsystem 132 may determine a quality score or quality score classification for a sample container based on an embedding generated for the image depicting the sample container. For example, an image of a sample container storing a subset of iPSCs may be obtained. The image may be tiled and provided to an embedding neural network, which may generate an embedding representing the morphological characteristics of the iPSCs depicted by the image. The embedding may be mapped to an embedding feature space.

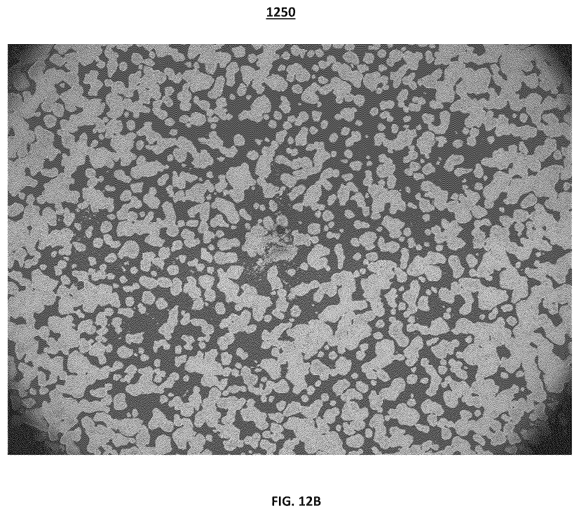

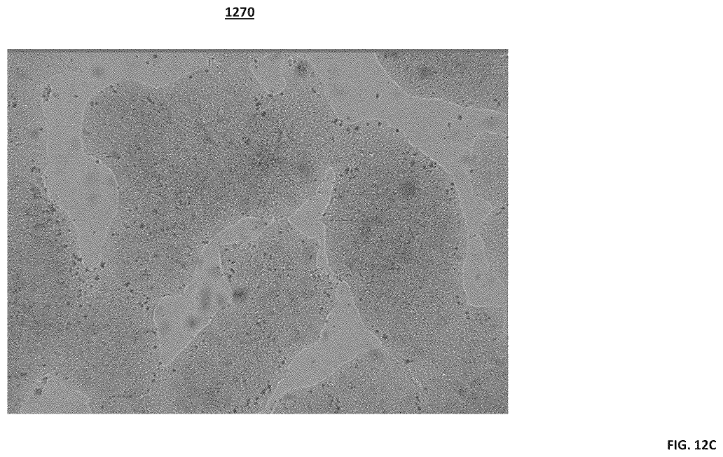

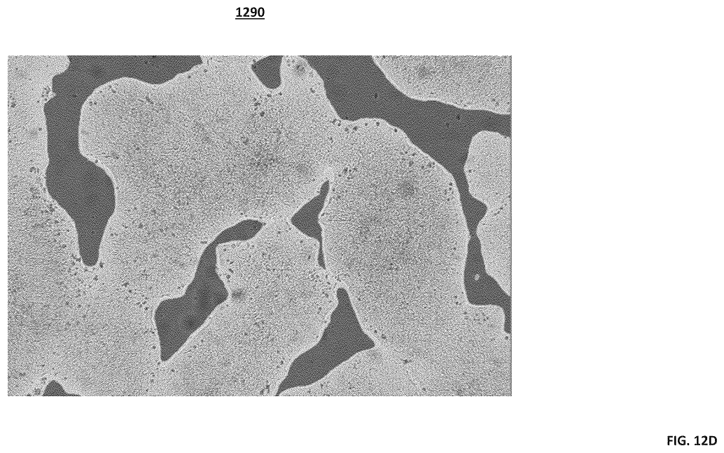

Confluence score determination subsystem 134 may be configured to determine a confluence score for each of the sample containers based on the captured images. The confluence score indicates a confluence of a subset of iPSCs within a sample container. Confluence indicates an amount of a sample container occupied by a subset of iPSCs. In some embodiments, confluence score determination subsystem 134 may use the same images as quality score determination subsystem 132. For example, the same images depicting the sample containers storing iPSCs may be imaged, and the images may be analyzed by quality score determination subsystem 132 and confluence score determination subsystem 134. As an example, with reference to FIGS. 12A-12D, images 1200 and 1270 may each depict a bright-field image of a sample container storing iPSCs. Images 1250 and 1290 may respectively depict images 1200 and 1270 including a confluence mask generated based on images 1200 and 1270. The confluence mask, as viewed by a user, may include pixels in a first color (e.g., white) representing portions of the sample container occupied by one or more alive iPSCs and pixels in a second color (e.g., black) representing portions of the sample container occupied by other contents (e.g., one or more dead iPSCs, debris, condensation, cell media not containing iPSCs, etc.). In some embodiments, confluence score determination subsystem 134 may employ one or more machine learning models stored in model database 152 to determine a confluence score for each of the sample containers based on the captured images. For example, a trained machine learning model (e.g., a U-Net) may analyze each of the images and determine a confluence score for each of the sample containers included, for example, on a multi-well plate. In some embodiments, the machine learning models may generate a mask representation for an image. The mask representation may then be used to compute the confluence score.

In some embodiments, confluence score determination subsystem 134 may classify the iPSCs stored in each sample container into one of a set of confluence classifications based on the confluence score for that sample container using the one or more machine learning models. For example, the confluence score for each sample container may be input to a classifier, which may assign a confluence classification to that sample container. In some embodiments, the iPSCs stored in a sample container may be classified into one of the confluence classifications based on whether the confluence score satisfies a confluence threshold condition. The confluence threshold condition may be satisfied if the confluence score is within a predefined range of confluence scores. In some embodiments, the set of confluence classifications may include: a first confluence classification and a second confluence classification. The first confluence classification may correspond to a confluence score that is within a predefined range of confluence scores (e.g., a confluence score that is greater than a first threshold confluence score and less than or equal to a second threshold confluence score), and the second confluence classification may correspond to a confluence score that is not within the predefined range of confluence scores (e.g., less than or equal to the first threshold confluence score or greater than the second threshold confluence score).

In some embodiments, the machine learning models may determine a confluence score for iPSCs stored within a sample container based on an embedding generated for the image depicting the sample container. The machine learning models may receive images depicting sample containers storing subsets of iPSCs and may generate an embedding for each image in a corresponding feature embedding space. As will be described in greater detail below, the image may be tiled (overlapping tiles or non-overlapping tiles), and an embedding may be produced for each tile. The embedding can be represented as a feature vector for the image. Confluence score determination subsystem 134 can use a neural network (e.g., a convolutional neural network) to generate a feature vector that represents each image. In some embodiments, the embedding neural network can be based on a ResNet image network trained on a dataset based on natural (e.g., non-medical) images, such as the ImageNet dataset. By using a non-specialized tile embedding network, confluence score determination subsystem 134 can leverage known advances in efficiently processing images to generate embeddings. Furthermore, using a natural image dataset allows the embedding neural network to learn to discern differences between segments of an image or a tile on a holistic level.

In some embodiments, confluence score determination subsystem 134 may determine the confluence score or confluence score classification for a sample container based on the machine learning model's generated mask representation. For example, the machine learning models may analyze a bright-field image and may generate a mask representation for that bright-field image. The mask representation may be generated using a UNET model, which outputs a mask in the same dimensions as the input image.

In some embodiments, confluence score determination subsystem 134 may determine a confluence score or confluence score classification for a sample container. For example, an image of a sample container storing a subset of iPSCs may be obtained. The image may be tiled and provided to an embedding neural network, which may generate an embedding representing the morphological characteristics of the iPSCs depicted by the image. The embedding may be mapped to an embedding feature space. The embedding informs a mask representation generated by the machine learning model.

In some embodiments, the embedding network used by quality score determination subsystem 132 may be the same or similar to the embedding network used by confluence score determination subsystem 134.

Sample container selection subsystem 136 may be configured to select one or more sample containers, and one or more maintenance operations, differentiation steps, or other actions, may be performed to the iPSCs stored within those sample containers. In some embodiments, the maintenance operations may include passaging on the subset of iPSCs stored within the at least one selected sample container, adding one or more reagents to the subset of iPSCs stored within the at least one selected sample container, banking the subset of iPSCs stored within the at least one selected sample container, performing a quality control check (e.g., classifying images of the iPSCs stored within sample containers into those storing “empty,” “low-quality,” “medium-quality,”or “high-quality” iPSCs) of the subset of iPSCs stored within the at least one selected sample container, performing a pluripotency status check of the subset of iPSCs stored within the at least one selected sample container, or discarding at least the subset of iPSCs stored within the at least one selected sample container. The iPSCs discarded may include only the subset of iPSCs stored within the at least one selected sample container, the iPSCs stored in multiple sample containers, or other the plurality of iPSCs stored within the first plurality of sample containers. The pluripotency status check may refer to a test to determine whether the iPSCs stored within a sample container have retained their pluripotency. Pluripotency status checks are described in, for example, WO 2022/261241 A1, the contents of which are herein incorporated by reference in their entirety. The iPSCs need to remain in their pluripotent state during cell maintenance to allow for cell differentiation. In some embodiments, the sample containers may be selected based on the quality score, confluence score, and/or the growth metric of the sample containers. In some embodiments, the sample containers may be selected based on the quality score classification, confluence score classification and/or the growth classification (e.g., positive or negative growth) of the sample containers. The selected sample containers may be subsequently passaged and, in some cases, subjected to one or more cell differentiation steps. In some embodiments, sample containers having medium-quality scores (e.g., a medium-quality classification) or high-quality scores (e.g., a high-quality classification) may be selected. In some embodiments, sample containers having a confluence score within a threshold range of confluence scores (e.g., having a first confluence classification) may be selected. In some embodiments, sample containers determined to be of (i) medium or high quality and (ii) within the threshold range of confluence scores may be selected.

In some embodiments, sample container selection subsystem 136 may be configured to perform passaging of the iPSCs stored within the selected sample containers. Passaging refers to a process whereby cells, such as induced pluripotent cells, are harvested, transferred to culture vessels with fresh growth medium, and used to start new cultures. Passaging may include one or more of the following: washing the subset of iPSCs, incubating the washed subset of iPSCs with a dissociation reagent, triturating the incubated subset of iPSCs after a media is added to the incubated subset of iPSCs, transferring the triturated subset of iPSCs to a sample container block, centrifuging the transferred subset of iPSCs in the sample container block to pellet the centrifuged subset of iPSCs, performing a buffering exchange to the pelleted subset of iPSCs by aspirating the pelleted subset of iPSCs, and suspending the aspirated subset of iPSCs into the media. Subsequent to passaging being performed, the passaged iPSCs stored within the selected sample containers may be subjected to one or more cell differentiation steps during the cell differentiation process.

In some embodiments, sample container selection subsystem 136 may be configured to select one or more sample containers, where iPSCs stored within the selected sample containers may be discarded. In some embodiments, iPSCs stored within these sample containers may be discarded based on the quality score and/or confluence score of those sample containers. For example, if a sample container is determined to have a quality score equal to the desired quality score, then sample container selection subsystem 136 may discard the subset of iPSCs stored within that sample container. In some embodiments, subsets of iPSCs stored in multiple sample containers of the multi-sample plate may be discarded based on the quality score and/or confluence score.

Model training subsystem 138 may be configured to train one or more machine learning models stored in model database 152 using training data stored in training data database 154. In some embodiments, the trained machine learning models may also be stored in model database 152. Model training subsystem 138 may be configured to generate and/or obtain the training data. For example, model training subsystem 138 may generate training data for training a machine learning model to determine a quality score of a sample container storing a subset of iPSCs based on an image depicting the sample container. Training data may be generated, for example in one embodiment, by collecting the data, cleaning and annotating the data, and performing the training (e.g., fitting the network to the data). In some embodiments, generating training data is supervised, such as by an individual. As another example, model training subsystem 138 may generate training data for training a machine learning model to determine a confluence score of a sample container storing a subset of iPSCs based on an image depicting the sample container. In some embodiments, the confluence score and the quality score may be determined using the same set of images depicting sample containers of iPSCs. The number of sample containers depicted within a given image may vary. For example, a multi-well plate may include four sample containers (or wells) each storing a subset of iPSCs and an image depicting these sample containers may be captured.

First differentiation stage subsystem 140 may be configured to cause the passaged iPSCs stored within the at least one selected sample container to be subject to one or more cell differentiation steps. In some embodiments, first differentiation stage subsystem 140 may be configured to cause the subset of iPSCs stored within the at least one selected sample container to be distributed across a second plurality of sample containers. First differentiation stage subsystem 140 may cause the iPSCs to be distributed across the second plurality of sample containers after passaging has been performed to the iPSCs. The number of sample containers in the second plurality of sample containers may be less than or equal to the number of sample containers included in the first plurality of sample containers. For example, the first plurality of sample containers may include twelve sample containers, while the second plurality of sample containers may include four sample containers. Each sample container of the second plurality of sample containers may include a substantially similar number of iPSCs. The cell differentiation steps may include feeding the iPSCs distributed within each sample container. The feeding may be performed by providing a particular media to the iPSCs to induce differentiation towards a particular type of cell (e.g., a motor neuron). In some embodiments, the feeding may be performed at a particular frequency, such as every hour, every few hours, every day, every week, etc. First differentiation stage subsystem 140 may cause the feeding to be performed at the desired frequency, however the frequency of the feedings may be adjusted during the differentiation steps.

In some embodiments, first differentiation stage subsystem 140 may be configured to cause a second plurality of images depicting the distributed subset of iPSCs in each of the second plurality of sample containers to be captured. For example, an imaging system may be used to capture the second plurality of images. First differentiation stage subsystem 140 may instruct the imaging system to capture the second plurality of images. In some embodiments, the first differentiation stage subsystem 140 may receive and analyze the second plurality of images to determine whether the iPSCs are developing as expected. For example, these images may be used for routine inspection to ensure that seeding is uniform, and that the iPSCs stored in the sample containers have a target cell density/confluence. Furthermore, these images may be used to ensure that no contaminants are present in the sample containers.

In some embodiments, first differentiation stage subsystem 140 may be configured to determine whether a first predefined amount of time has elapsed from the feeding of the distributed subsets of iPSCs in each of the second plurality of sample containers. If the first predefined amount of time has not elapsed, first differentiation stage subsystem 140 may cause another feeding to the subset of iPSCs. However, if the first predefined amount of time has elapsed, the first differentiation stage subsystem 140 may cause the distributed subset of iPSCs in each of the second plurality of sample containers to be banked in cell storage. The iPSCs stored in the banked second plurality of sample containers may be referred to as a “lot” of iPSCs. In some embodiments, the first predefined amount of time may be three days, however other amounts of time may be used (e.g., 1 or more days, 5 or more days, 10 or more days, etc.). The amount of time may be selected such that progenitors can sufficiently expand. The sufficient expansion of the progenitors may enable the banked iPSCs to be frozen without the iPSCs becoming too differentiated for survival following the freezing and replating during subsequent differentiation steps. Banking the lot of iPSCs may include freezing the iPSCs at a temperature that inhibits cell growth and cell death.

In some embodiments, first differentiation stage subsystem 140 may be configured to assess a quality of a differentiation as it proceeds. First differentiation stage subsystem 140 may implement a machine learning model trained to assess the quality of the differentiation. The machine learning model may be stored in model database 152. This machine learning model may be cell type specific. As an example, to train a machine learning model to assess the differentiation for motor neurons, a number of motor neuron differentiations may be performed to obtain training data describing failed and successful differentiations. The training data may be stored in training data database 154. The training data may include the failed/successful differentiation information matched with quantitative phase-contrast (QPC) imaging or bright-field imaging. The training data may be used to train a machine learning model to predict whether a differentiation step will be successful as the differentiation is occurring.

Quality control (QC) check subsystem 142 may be configured to cause a QC check to be performed to one or more subsets of iPSCs. For example, QC check subsystem 142 may select a sample container from the second plurality of sample containers. A QC check may be performed to the iPSCs stored within the selected sample container of the second plurality of sample containers. The QC check may serve as a proxy for the lot of iPSCs stored in the banked sample containers. The QC check may involve staining the iPSCs. iPSCs that have been stained cannot be differentiated. Therefore, to prevent damaging the lot of iPSCs, the iPSCs from one sample container may be selected. If these iPSCs pass the QC check, then the lot of iPSCs may be released for further cell differentiation. However, if these iPSCs do not pass the QC check, quality control check subsystem 142 may be configured to cause the lot of iPSCs to be discarded. In some embodiments, QC check subsystem 142 may randomly select a sample container from the second plurality of sample containers for which to perform the QC check.

In some embodiments, the iPSCs stored within the selected sample container from the second plurality of sample containers may be thawed and fed. Thawing the iPSCs may include placing vials of the iPSCs in a water bath (e.g., at 37° C.). The contents of the vial may be transferred to a block of sample containers, and the vial may be washed with a cell-type specific media. The block of sample containers may be centrifuged in the liquid in the block and then aspirated. The cell-type specific media may then be added to resuspend the iPSCs. In some embodiments, a cell count may be performed. The cell count may be performed manually and/or using one or more machine learning models. For example, a computer vision model trained to detect iPSCs may be used to estimate a quantity of iPSCs that have been resuspended. Based on the estimated quantity of iPSCs, a concentration of the iPSCs and media may be adjusted to meet requirements for plating/seeding. The iPSCs (e.g., the iPSCs from the selected sample container of the second plurality of sample containers that has been resuspended) may then be seeded in a third plurality of sample containers of, for example, a multi-well plate.

In some embodiments, QC check subsystem 142 may be configured to cause the iPSCs stored in the third plurality of sample containers to be fed. These iPSCs may be fed at a predefined frequency for cell differentiation. For example, the iPSCs may be fed every two days up until a second threshold amount of time has elapsed (e.g., 10 days). In some embodiments, the iPSCs may be fed using 80 micro-liters of media aspirated from the sample containers. Furthermore, 100 micro-liters of fresh media may be dispensed into the sample containers.

In some embodiments, QC check subsystem 142 may be configured to cause a third plurality of images of the iPSCs stored in the third plurality of sample containers to be captured. In some embodiments, QC check subsystem 142 may receive and analyze the third plurality of images to determine that the iPSCs are developing as expected. For example, these images may be used for routine inspection to ensure that seeding is uniform, and that the iPSCs stored in the sample containers have a target cell density/confluence. Furthermore, these images may be used to ensure that no contaminants are present in the third plurality of sample containers.

In some embodiments, QC check subsystem 142 may be configured to determine whether a second predefined amount of time has elapsed from the feeding of the iPSCs stored within the selected sample container. As an example, the second predefined amount of time may be 10 days, however other amounts of time may be used. 10 days may represent a typical amount of time that iPSCs need to be fed at a particular cadence in order to obtain differentiated cells that can be analyzed using biomarkers for quality. If QC check subsystem 142 determines that the second predefined amount of time has not elapsed, then QC check subsystem 142 may cause the feeding and image capturing of iPSCs stored within the sample container selected for the QC check to be repeated. For example, an additional round of feeding may be performed to the iPSCs stored in the third plurality of sample containers, after which imaging and a check of whether the second predefined amount of time elapsed may be performed. If QC check subsystem 142 determines that the second predefined amount of time has elapsed, then QC check subsystem 142 may cause a QC check to the iPSCs stored in the third plurality of sample containers.

In some embodiments, QC check subsystem 142 may be configured to cause a QC check to be performed on the iPSCs stored within the selected sample container based on the second predefined amount of time having elapsed. The QC check may produce a QC score. The QC score may indicate whether the iPSCs stored in the selected sample container satisfy QC standards. The QC checks may include applying one or more stains to the sampled iPSCs, imaging the stained iPSCs, and determining whether those imaged iPSCs pass the QC, such as comparing the imaged iPSCs with a pre-determined threshold percentage of the total number of iPSCs that are positive for a marker of interest specific to a particular cell type. In some embodiments, QC check subsystem 142 may cause the iPSCs stored in the selected sample container, as well as some or all of the lot of iPSCs, to be discarded based on the QC score being less than a threshold QC score. In some embodiments, QC check subsystem 142 may cause the lot of iPSCs banked in cell storage to be released based on the QC score being greater than or equal to the threshold QC score. After the lot of iPSCs has been released, they may be subjected to one or more additional cell differentiation steps to produce the desired differentiated cells. The lot of iPSCs, as mentioned above, may include the iPSCs stored in the second plurality of sample containers excluding the selected sample container, for which the QC check was performed.

Second differentiation stage subsystem 144 may be configured to cause one or more additional cell differentiation steps to be performed to the lot of iPSCs to obtain a plurality of differentiated cells. The differentiated cells may be of a particular cell type desired for phenotyping. For example, the differentiated cells may be motor neurons, which can be used for clinical trials related to treatment of neurological conditions.

In some embodiments, second differentiation stage subsystem 144 may be configured to cause the lot of iPSCs stored within the second plurality of sample containers to be distributed across a third plurality of sample containers. Second differentiation stage subsystem 144 may cause the lot of iPSCs to be distributed across the third plurality of sample containers after the QC check has been performed to the iPSCs stored in the selected sample container from the second plurality of sample containers. The number of sample containers in the third plurality of sample containers may be less than or equal to the number of sample containers included in the first plurality of sample containers and/or the second plurality of sample containers. Each sample container of the third plurality of sample containers may include a substantially similar number of iPSCs. In some embodiments, second differentiation stage subsystem 144 may be configured to cause the distributed subset of iPSCs in each of the third plurality of sample containers to be subjected to one or more additional cell differentiation steps. The cell differentiation steps may include feeding the iPSCs distributed within each sample container of the third plurality of sample containers. The feeding may be performed by providing a particular media to the iPSCs to induce differentiation towards a particular type of cell (e.g., a motor neuron). In some embodiments, the feeding may be performed at a particular frequency, such as every hour, every few hours, every day, every week, etc. In some embodiments, the feeding may include an additional compound, such as small molecules. For example, a given sample container of the third plurality of sample containers may already include iPSCs. The small molecules may be added to the sample container to elicit a phenotyping response.

In some embodiments, second differentiation stage subsystem 144 may be configured to cause a fourth plurality of images depicting the distributed lot of iPSCs in each of the third plurality of sample containers to be captured. For example, an imaging system may be used to capture the fourth plurality of images. In some embodiments, second differentiation stage subsystem 144 may receive and analyze the fourth plurality of images to determine that the iPSCs are developing as expected. For example, these images may be used for routine inspection to ensure that seeding is uniform, and that the iPSCs stored in the sample containers have a target cell density/confluence. Furthermore, these images may be used to ensure that no contaminants are present in the sample containers.

In some embodiments, second differentiation stage subsystem 144 may be configured to determine whether a third predefined amount of time has elapsed from the feeding of the distributed subsets of iPSCs in each of the third plurality of sample containers. If the third predefined amount of time has not elapsed, second differentiation stage subsystem 144 may cause another feeding to the iPSCs. However, if the third predefined amount of time has elapsed, second differentiation stage subsystem 144 may cause the differentiated cells to a phenotyping system. In some embodiments, the third predefined amount of time may be the same or similar to the second predefined amount of time (e.g., 10 days). The amount of time may be selected such that progenitors can sufficiently expand.

In some embodiments, second differentiation stage subsystem 144 may be configured to assess a quality of a differentiation as it proceeds. Second differentiation stage subsystem 144 may implement a machine learning model trained to assess the quality of the differentiation. The machine learning model may be stored in model database 152. This machine learning model may be cell type specific. As an example, to train a machine learning model to assess the differentiation for motor neurons, a number of motor neuron differentiations may be performed to obtain training data describing failed and successful differentiations. The training data may be stored in training data database 154. The training data may include the failed/successful differentiation information matched with imaging QPC or bright-field imaging. The training data may be used to train a machine learning model to predict whether a differentiation step will be successful as the differentiation is occurring. In some embodiments, the machine learning models used to determine the quality of the differentiation may be the same or similar for both first differentiation stage subsystem 140 and second differentiation stage subsystem 144.

FIG. 1C illustrates an exemplary sample ranking scheme for selecting samples, in accordance with some embodiments. For each sample, an exemplary system (e.g., one or more electronic devices) determines a confluence score, a quality score, and a growth metric. Based on the confluence score, the quality score, and the growth metric of each sample, the system can rank the samples according to the sample ranking scheme in FIG. 1C. After ranking the samples, the system can select a top ranked subset of sample for maintenance operations (e.g., passaging). In some embodiments, the system can select a predefined number of samples (e.g., top 100 samples of the ranked samples). In some embodiments, the system can select a predefined percentage of samples (e.g., top 10% of the ranked samples). In some embodiments, the system can select all samples above or meeting a set of criteria in the sample ranking scheme. For example, the system can select all samples meeting the set of criteria 168 in FIG. 1C as well as all samples ranked higher than these samples.

The confluence score for each sample represents a confluence of the iPSCs distributed within the corresponding sample container. As described herein, the confluence score may be a numerical value such as a percentage. As shown in FIG. 1C, the sample ranking scheme can specify a ranking of a plurality of predefined confluence score ranges, such as R1, R2, . . . . Rn, etc. Each of R1, R2, . . . . Rn, etc. represents a range of confluence scores having an upper threshold and a lower threshold. In some embodiments, the ranking of the confluence score ranges can include, from high to low: 60-64%, 65-69%, 70-74%, 55-59%, 50-54%, 45-49%, 40-44%, 35-39%, 30-34%, 25-29%, 20-24%, 15-19%, 75-79%, 80-84%, 85-89%, 10-14%, 5-9%, 90-94%, 95-99%, 100%, 0-4%. For example, R1 in FIG. 1C may be 60-64%, R2 in FIG. 1C may be 65-69%, etc. Additionally or alternatively, the predefined confluence score ranges can include about 50% to about 80%, from about 60% to about 80%, and/or from about 65% to about 75%.

The quality score for each sample represents a quality of the iPSCs distributed within the corresponding sample container. The quality score can be a binary value, a numerical value, or a classification. For example, the quality score can be one of: high quality, medium quality, low quality. As another example, the quality score can be a numerical value on a scale of 1 to 10, 1 to 100, 1 to 1000, or the like. As another example, the quality score can be a relative value such as a percentile value (e.g., top 1 percentile, top 2 percentile, or the like). As another example, the quality score can be a numerical value such as a percentage. As another example, the quality score can be a grade such as A, B, C, D, or the like. Further, each numerical score can be translated into a classified score. For example, for numerical scores on the scale of 1-100, a numerical score above a first threshold (e.g., 67-100) can be translated into a classification of high quality; a numerical score below the first threshold and above a second threshold (e.g., 34-66) can be translated into a classification of medium quality; a numerical score below the second threshold (e.g., 0-33) can be translated into a classification of low quality. One of ordinary skill should appreciate that the quality score can be of any value indicative of absolute or relative quality of a sample. In the depicted example in FIG. 1C, the quality score is one of: high, medium, and low.

The growth metric for each sample represents a growth status of the iPSCs distributed within the corresponding sample container. The growth metric can be a binary value, a numerical value, or a classification. In some embodiments, the growth metric is indicative of whether the iPSCs of the plurality of iPSCs distributed within each sample container is in a growth phase. For example, the growth metric can be one of two classifications: in growth phrase and not in growth phase. As another example, the growth metric can be one of two classifications: positive (i.e., in growth phase) and negative (i.e., not in growth phase). As another example, the growth metric can be a numerical value indicative of a growth rate. If the numerical value exceeds a threshold (e.g., 0), the growth metric indicates positive growth. As discussed herein, the system can rank a sample having a higher or positive growth metric higher than a sample having a lower or negative growth metric.

In some embodiments, the growth metric of a sample can be determined based on the confluence scores of the sample at different time points. For example, the system can obtain a first confluence score and a second confluence score of the sample, with the first confluence score associated with a first time point and the second confluence score associated with a second time point later than the first time point. If the second confluence score is higher than the first confluence score, the growth metric is set to indicate positive growth (or that the sample is in the growth phase). For example, the system can determine that the confluence score of the sample is 65% yesterday and the confluence score of the sample is 80% today, and the increase in confluence score indicates that the sample is in positive growth. It should be appreciated that the growth metric can be determined in other manners, such as by monitoring and comparing (automatically or manually) the state of the sample (e.g., density, weight, volume, etc.) over time. The monitoring of the state of the sample can be done via machine-learning models, such as machine-learning models configured to receive data related to the sample (e.g., one or more images) and output an evaluation of the sample (e.g., estimated density).

With reference to the sample ranking scheme in FIG. 1C, as shown by 162, 166, and 168, for samples in the same confluence score range (e.g., R1) and having a positive growth rate, a sample having a high quality score is ranked higher than a sample having a medium quality score (as shown by 162 and 166), which is ranked higher than a sample having a low quality score (as shown by 166 and 168).

Further with reference to the sample ranking scheme in FIG. 1C, as shown by 162 and 164, the predefined confluence score range R1 is higher than the predefined confluence score range R2. Accordingly, in the depicted example, a sample having a confluence score in the range R1 is ranked higher than a sample having a confluence score in the range R2 if they have the same growth rate and the same quality score (e.g., as shown by 162 and 164).

Further with reference to the sample ranking scheme in FIG. 1C, as shown by 164 and 166, a sample having a confluence score in a worse predefined confluence score range (e.g., R2) and a higher quality score (e.g., high) may be ranked higher than a sample having a confluence score in a better predefined confluence score range (e.g., R1) and a lower quality score (e.g., M).

Further with reference to the sample ranking scheme in FIG. 1C, all samples having a positive growth rate are ranked higher than all samples having a negative growth rate. Accordingly, in the depicted example, a sample having a positive growth rate (regardless of confluence score or quality score) is ranked higher than a sample having a negative growth rate (regardless of confluence score or quality score).

Further with reference to the sample ranking scheme in FIG. 1C, the ranking scheme can specify that samples in a first group of confluence score ranges are ranked higher than the samples in a second group of confluence score ranges, regardless of the quality score. For example, for all samples having confluence score ranges R1-Rn (with positive growth rate) are ranked higher than all samples Rₙ₊₁-Rₙ₊ₘ (with positive growth rate), regardless of the quality score.

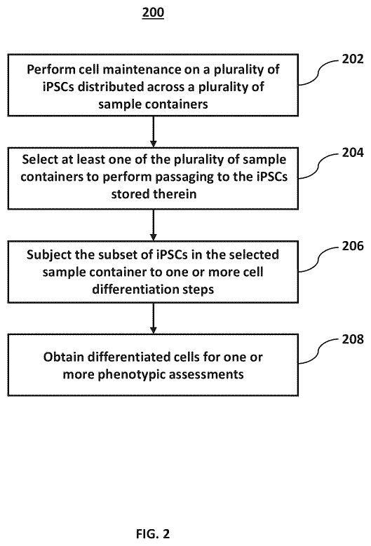

FIG. 2 illustrates an exemplary method 200 for autonomously maintaining and differentiating iPSCs to obtain differentiated cells, in accordance with some embodiments. Method 200 is performed, for example, using one or more electronic devices implementing a software platform, such as AMD platform 100. In some examples, method 200 is performed using a client-server system, and the steps of method 200 are divided up in any manner between the server (e.g., AMD platform 100) and one or more client devices (e.g., client device 102). Thus, while portions of method 200 are described herein as being performed by particular devices of a client-server system, it will be appreciated that method 200 is not so limited. In other examples, method 200 is performed using only a client device or only multiple client devices. In method 200, some blocks are, optionally, combined, the order of some blocks is, optionally, changed, and some blocks are, optionally, omitted. In some examples, additional steps may be performed in combination with method 200. Accordingly, the operations as illustrated (and described in greater detail below) are exemplary by nature and, as such, should not be viewed as limiting.

Method 200 may begin at operation 202. In operation 202, an exemplary system (e.g., one or more electronic devices) may perform a cell maintenance process to a plurality of iPSCs distributed across a first plurality of sample containers. For example, maintenance processing system 110 may perform cell maintenance processes to iPSCs distributed across sample containers of a multi-sample plate (e.g., a plate having four or more sample wells, a plate having twelve or more sample wells, etc.). Each sample container may contain a subset of iPSCs. During the cell maintenance process, images depicting the multi-well plate may be captured by a digital microscopy imaging system. The captured images may be input to one or more machine learning models, 132 and 134, which may be trained to determine a quality score and/or a confluence score of each sample container. Depending on the quality and/or confluence scores, maintenance processing system 110 may determine a subsequent action to be performed to the iPSCs.

In operation 204, at least one sample container of the first plurality of sample containers may be selected for passaging. In some embodiments, maintenance processing system 110 may use the machine learning models, 132 and 134, to determine which subsequent action(s) to perform on the selected sample containers. The machine learning models may be trained to determine a quality score and/or a confluence score for each sample container based on the captured images. Maintenance processing system 110 may determine whether the quality score, the confluence score, and/or the growth metric of the sample containers satisfy a quality threshold condition, a confluence threshold condition, and a growth metric threshold, respectively. The quality threshold condition may be satisfied if a quality score for a sample container is greater than or equal to a threshold quality score. The confluence threshold condition may be satisfied if a confluence score for a sample container is within a predefined range of confluence scores. The growth metric threshold may be satisfied if the growth metric for a sample container indicates positive growth rate (i.e., that the sample is in a growth phase).

Maintenance processing system 110 may discard iPSCs stored sample containers determined to have a quality score that does not satisfy the quality threshold condition. For example, a sample container having a low-quality score or empty quality score (e.g., less than a threshold number of iPSCs) may be discarded. Maintenance processing system may perform one or more additional feedings to iPSCs stored in sample containers determined to have a quality score that does not satisfy the quality threshold condition and the confluence threshold condition. For example, a sample container having a medium-quality score may have one or more additional feedings performed to the iPSCs stored therein. As another example, a sample container having a confluence score outside of the predefined range of confluence scores may have one or more feedings performed to the iPSCs stored therein. In this example, the sample containers may be re-imaged, and the newly captured images may be input to the machine learning models to compute an updated quality score and an updated confluence score for those sample containers.

Maintenance processing system 110 may perform passaging of iPSCs in sample containers determined to satisfy the quality threshold condition, the confluence threshold condition, and/or the growth metric threshold. For example, maintenance processing system 110 may select one or more sample containers having a high-quality score and/or a confluence score within the threshold range for passaging. The subsets of iPSCs stored within these sample containers may be passaged. In some embodiments, passaging may include: washing a subset of iPSCs in each of the selected sample containers, incubating the washed subset(s) of iPSCs, triturating the incubated subset(s) of iPSCs after a media is added to the incubated subset(s) of iPSCs, transferring the triturated subset(s) of iPSCs to a sample container block, centrifuging the transferred subset(s) of iPSCs in the sample container block to pellet the centrifuged subset(s) of iPSCs, performing a buffering exchange to the pelleted subset(s) of iPSCs by aspirating the pelleted subset of iPSCs, and suspending the aspirated subset(s) of iPSCs into the media. In some embodiments, after passaging has been performed, the selected sample containers may be provided to a cell storage until cell differentiation steps are to be performed.

In operation 206, differentiation processing system 120 may subject the subset of iPSCs within the selected sample containers to one or more cell differentiation steps. The cell differentiation steps may include thawing and feeding the selected sample containers, imaging the iPSCs, and analyzing the iPSCs. The analysis may cause differentiation processing system 120 to perform one or more additional feedings to the iPSCs stored within the selected sample containers. Alternatively, differentiation processing system 120 may bank (e.g., store) the iPSCs quality control checks. For example, a determination may be made as to whether a predefined amount of time for the iPSCs to mature has elapsed (e.g., 3 days). If so, the iPSCs stored in the selected sample containers may be banked in cell storage. The banked iPSCs may be stored in cell storage, which may include cooling the cells down to a predefined temperature (e.g., −195.8° C.). If not, the iPSCs stored within the selected sample containers may be fed. After banking the iPSCs stored in the selected sample containers, a small sample of the iPSCs may be retrieved for performing quality control checks. The quality control checks may include applying one or more stains to the sampled iPSCs, imaging the stained iPSCs, and determining whether those imaged iPSCs pass the quality checks, such as comparing the imaged iPSCs with a pre-determined threshold percentage of the total number of iPSCs that are positive for a marker of interest specific to a particular cell type. If so, differentiation processing system 120 may release the lot of iPSCs still banked in the cell storage system for further cell differentiation steps/phenotyping. If not, differentiation processing system 120 may discard some or all of the iPSCs still banked (as sample container as the iPSCs that have been stained).

In operation 208, differentiated cells may be obtained for one or more phenotypic assessments. The released lot of iPSCs may be thawed, fed, imaged, and allowed to mature. After a predefined amount of time has elapsed for the cells to mature as needed (e.g., 10 days), the differentiated cells may be obtained. Different assays or other phenotypic analyses may then be performed using the differentiated cells. Different cell types may be produced as a result of the differentiations. For example, neurons, skin cells, skeletal muscle cells, renal cells, and/or other types of cells may be obtained.

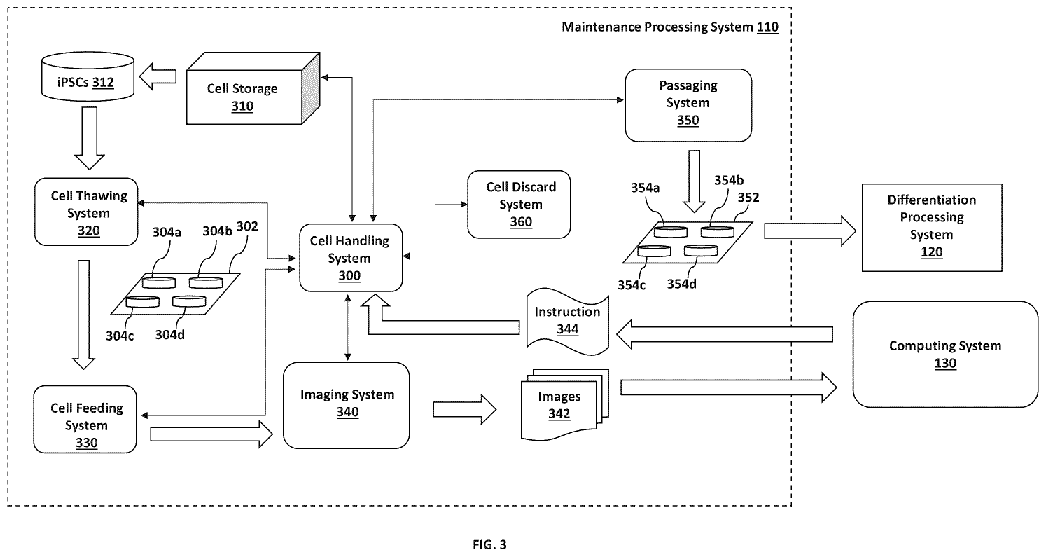

FIG. 3 illustrates an example maintenance processing system 110 for performing maintenance of iPSCs, in accordance with some embodiments. Maintenance processing system 110 may include a cell handling system 300, a cell storage 310, a cell thawing system 320, a cell feeding system 330, a microscopy imaging system 340, a passaging system 350, a cell discard system 360, or other components. Maintenance processing system 110 may communicate with differentiation processing system 120, computing system 130, or other components of system 10 (shown in FIG. 1A) to perform one or more evaluations and/or facilitate one or more actions relating to the process of maintaining iPSCs (e.g., discarding iPSCs, feeding iPSCs, imaging iPSCs, passaging iPSCs, etc.).

Cell handling system 300 may be configured to transport iPSCs to various components of maintenance processing system 110. Cell handling system 300 may include one or more robotic devices that receive instructions from computing system 130 or other components of system 10 (e.g., client device 102 of FIG. 1A). Instructions 344 for controlling the movements of the robotic devices of cell handling system 300 may be generated by computing system 130. The instructions may cause the robotic devices of cell handling system 300 to perform various actions, such as, but not limited to, retrieving iPSCs 312 from cell storage 310, providing iPSCs 312 to cell thawing system 320, distributing iPSCs 312 across a plurality of sample containers 304a-304d of a multi-sample or multi-well plate 302, providing multi-sample or multi-well plate 302 to cell feeding system 330 for feeding iPSCs 312, providing multi-sample or multi-well plate 302 to imaging system 340 for capturing images of iPSCs 312 stored in sample containers 304a-304b (collectively “sample containers 304”), selecting a subset or subsets of iPSCs 312 stored in sample containers 304, providing the selected sample container(s) to passaging system 350 for passaging the subset or subsets of iPSCs 312 stored within the selected sample containers 304, providing one or more sample containers of iPSCs 312 to cell discard system 360 to be discarded, or to have other actions performed to iPSCs 312. In some embodiments, cell handling system 300 may provide a multi-sample or multi-well plate 352 including sample containers or wells 354a-354d (collectively “sample containers 354”) storing passaged iPSCs 312 to differentiation processing system 120.

Although a single cell handling system 300 is depicted in maintenance processing system 110, it should be understood that multiple robotic devices may be implemented by cell handling system 300. These robotic devices may perform various actions using the components of maintenance processing system 110. For example, cell handling system 300 may include a robotic device configured to retrieve iPSCs 312 from cell storage 310 and distribute iPSCs 312 across sample containers 304, a robotic device configured to transport sample containers 304 to cell thawing system 320, etc. Cell handling system 300 may also perform tasks conventionally performed by human operators.

Cell storage 310 may be configured to store iPSCs, including iPSCs 312, to be developed into differentiated cells. In some embodiments, cell storage 310 may store different iPSCs to be developed into different types of differentiated cells. For example, cell storage 310 may store a batch of iPSCs to be developed into motor neurons whereas another, separate sample container (not shown) may contain a separate batch of iPSCs to be developed into renal cells. Cell storage 310 may be configured to store iPSCs at a particular temperature to preserve the iPSCs for future use. For example, cell storage 310 may operate at a temperature of −195.8° C., however other temperatures may be used. Within cell storage 310, the batches of iPSCs may be stored in one or more vials designed to safely store and track iPSCs.

In some embodiments, cell handling system 300 may retrieve iPSCs 312 from cell storage 310. An instruction 344 may be generated by computing system 130 and provided to cell handling system 300 indicating which iPSCs 312 to retrieve. In some embodiments, instruction 344 may be generated by computing system 130 based on a command provided by client device 102. For example, a user may indicate a batch or batches of iPSCs 312 to retrieve from cell storage 310. Instruction 344 may cause cell handling system 300 to retrieve vials storing iPSCs 312 from cell storage 310. In some embodiments, a user may select which iPSCs 312 to retrieve from cell storage 310. The user may input a command to AMD platform 100 using client device 102. The command may specify the iPSCs to retrieve from cell storage 310 (e.g., iPSCs 312). For example, the command may cause a robotic device of cell handling system 300 to physically retrieve iPSCs 312 from cell storage 310. In some embodiments, iPSCs 312 may include 1,000 or more iPSCs, 10,000 or more iPSCs, 100,000 or more iPSCs, 1,000,000 or more iPSCs, 10,000,000 or more iPSCs, 100,000,000 or more iPSCs, or other quantities.

Cell handling system 300 may provide iPSCs 312, retrieved from cell storage 310, to cell thawing system 320. Cell thawing system 320 may be configured to thaw iPSCs 312. In some embodiments, a thawing media is used to thaw iPSCs 312. For example, a cGMP, stabilized feeder-free maintenance media may be used to thaw iPSCs 312. In some embodiments, the thawing media may include an inhibitor. For example, the thawing media used by cell thawing system 320 may be a selective small molecule inhibitor of Rho-associated kinase (ROCK). Cell thawing system 320 may thaw iPSCs 312 in the presence of the thawing media containing the inhibitor (e.g., a ROCK inhibitor) for a predefined amount of time (e.g., one day), after which the cell culture media may be changed. For example, cell thawing system 320 may thaw iPSCs 312 in the presence of the cell culture thawing media containing the inhibitor, and the next day the media may be changed to just the cell culture thawing media without the inhibitor for cell growth and maintenance.

In some embodiments, cell handling system 300 may be configured to distribute iPSCs 312 across sample containers 304 after iPSCs 312 have been thawed by cell thawing system 320. Multi-sample or multi-well plate 302 may include a first plurality of sample containers or wells 304. For example, multi-sample or multi-well plate 302 may include 4 sample containers or wells, 16 sample containers or wells, 32 sample containers or wells, 64 sample containers or wells, 96 sample containers or wells, or other quantities of sample containers or wells. In the illustrated embodiment, multi-sample or multi-well plate 302 is depicted as including 4 sample containers or wells for illustrative purposes. In some embodiments, each of sample containers or wells 304 may include a respective subset of iPSCs 312. Each subset of iPSCs 312 may be substantially similar to one another. For example, iPSCs 312 may include 1,000 iPSCs, retrieved from cell storage 310, and each of sample containers or wells 304 may store approximately 250 iPSCs. However, each subset of iPSCs 312 may vary in size. For example, sample container or well 304a may store 5×10⁵ iPSCs, sample container or well 304b may store 3×10⁵ iPSCs, sample container or well 304c may store 1.5×10⁵ iPSCs, and sample container or well 304d may store 5×10⁴ iPSCs.

Cell handling system 300 may be configured to provide multi-sample or multi-well plate 302, which may include iPSCs 312 distributed across sample containers 304, to cell feeding system 330. Cell feeding system 330 may be configured to perform one or more feedings to each subset of iPSCs 312 stored within sample containers or wells 304. To feed each subset of iPSCs 312, cell feeding system 330 may be configured to aspirate old spent cell culture media (e.g., 1,000 μL) from each of sample containers or wells 304 using an automated liquid handler, which may then be disposed. Fresh cell culture media may be added by transferring, using the automated liquid handler of cell feeding system 330, from a reservoir to each of sample containers or wells 304. In some embodiments, the cell culture media used by cell feeding system 330 to feed each subset of iPSCs 312 may be the same or similar to the media used for thawing. For example, a cGMP, stabilized feeder-free maintenance media may be used to feed iPSCs 312. In some embodiments, cell feeding system 330 may be configured to incubate each subset of iPSCs 312 at a particular temperature (e.g., 37° C.). Cell feeding system 330 may feed each subset of iPSCs 312 by exchanging cell culture media at a predefined cadence. For example, the cell culture media may be exchanged every 24 hours.

After cell feeding system 330 performs the feedings, cell handling system 300 may provide multi-sample or multi-well plate 302 to imaging system 340. In some embodiments, imaging system 340 may be a digital microscopy imaging system configured to capture digital images. In some embodiments, the images may be bright-field, phase contrast, or fluorescent images. Imaging system 340 may be configured to capture a plurality of images 342 depicting sample containers or wells 304 storing each subset of iPSCs 312 subsequent to cell feeding system 330 performing the feedings. In some embodiments, images 342 may be whole slide images depicting some or all of sample containers or wells 304. For example, the whole slide images may be 100,000×100,000 pixels. As another example, images 342 may each be JPG files of 1224×904 pixels. In some embodiments, images 342 of sample containers or wells 304 may be captured at different magnification levels (e.g., 5× magnification, 10× magnification, 20× magnification, etc.). In some embodiments, each of images 342 may be divided into a plurality of tiles. The tiles may be of a smaller size (e.g., 512×512 pixels), which may facilitate faster computations. A number of images 342 and a resolution, magnification, etc., may vary and may be determined based on instructions 344 provided to imaging system 340 from computing system 130. In some embodiments, 4×4 (16 field of views total) may be captured at a particular magnification level (e.g., 4× magnification, 10× magnification, etc.). For example, imaging system 340 may capture bright-field images at 14× magnification. An example bright-field image 1200 is depicted in FIG. 12A. In particular, bright-field image 1200 may depict an entire sample container storing iPSCs.

In some embodiments, imaging system 340 may provide images 342 to computing system 130 for analysis. Computing system 130 may be configured to determine a quality score, a confluence score, a growth metric, and/or other metrics describing morphological features of each subset of iPSCs 312 stored in each of sample containers 304. As previously shown, computing system 130 may include quality score determination subsystem 132 and confluence score determination subsystem 134. Quality score determination subsystem 132 may be configured to determine a quality score for each of sample containers or wells 304 based on images 342. Quality score determination subsystem 132 may further be configured to determine whether the quality scores of sample containers or wells 304 satisfy a quality threshold condition. Confluence score determination subsystem 134 may be configured to determine a confluence score for each of sample containers or wells 304 based on images 342. Confluence score determination subsystem 134 may further be configured to determine whether the confluence scores of sample containers or wells 304 satisfy a confluence threshold condition. In some embodiments, the confluence score determinations subsystem 134 may be used to determine a growth metric of each of sample containers or wells 304, as described herein.

In some embodiments, quality score determination subsystem 132 receives images 342 from imaging system 340. As mentioned above, images 342 may depict iPSCs 312 distributed across sample containers or wells 304 of multi-sample or multi-well plate 302, where each of the sample containers or wells 304 may store a subset of iPSCs 314. Quality score determination subsystem 132 may determine a quality score for each of sample containers or wells 304 based on images 342. In some embodiments, quality score determination subsystem 132 may use one or more machine learning models to compute a quality score for each of sample containers or wells 304. For example, a quality score model may be trained to compute a quality score for a sample container where the quality score indicates a quality of the iPSCs stored within the sample container or well. In some embodiments, the quality score may be a continuous value (e.g., a numerical value between 0-100). Alternatively, or additionally, quality score determination subsystem 132 may generate a discrete quality score (e.g., classifying each sample container into one of a set of quality classifications). For example, the one or more machine learning models may include a convolutional neural network (CNN) trained to analyze images 342 and output a quality score for each of sample containers or wells 304, and the quality scores may be passed to a classifier trained to classify each of sample containers or wells 304 into one of a set of quality classifications based on the quality score of that sample container or well.

Quality score determination subsystem 132 may classify, using the one or more machine learning models, the quality score into one of a set of a quality classification. The set of quality classifications may include:
(i) an empty classification indicating that a sample container of sample containers or wells 304 is empty of iPSCs (e.g., less than a threshold number of iPSCs are present in the sample container),
(ii) a low-quality classification indicating that the machine learning models computed that the subset of iPSCs stored within the sample container or well has a low-quality score,
(iii) a medium-quality classification indicating that the machine learning models computed that the subset of iPSCs stored within the sample container or well has a medium-quality score, and
(iv) a high-quality classification indicating that the machine learning models computed that the subset of iPSCs stored within the sample container or well has a high-quality score.

In some embodiments, the low-quality score refers to a quality score that is less than a first quality threshold score. In some embodiments, the medium-quality score refers to a quality score that is greater than or equal to the first quality threshold and less than a second quality threshold score. In some embodiments, the high-quality score refers to a quality score that is greater than or equal to the second quality threshold score.

In some embodiments, a confluence score determination subsystem 134 receives images 342 from imaging system 340. As mentioned above, images 342 may depict the status of iPSCs 312 distributed across sample containers or wells 304 of multi-sample or multi-well plate 302, where each of sample containers or wells 304 may store a subset of iPSCs 312. Confluence score determination subsystem 134 may determine a confluence score for each of sample containers or wells 304 based on images 342. The confluence score represents a confluence of iPSCs within a given sample container. The confluence indicates an amount of the sample container or well that is occupied by iPSCs. In some embodiments, confluence score determination subsystem 134 may generate a mask representation for each of images 342. A trained machine learning model may be used to generate the mask representation, which are generated in binary. The mask representation may represent pixels from the bright-field image (e.g., images 342) depicting one or more alive iPSCs in one manner and pixels depicting other contents (e.g., one or more dead iPSCs, debris, condensation, cell media not containing iPSCs, etc.) in another manner. For example, with reference to FIGS. 12A-12D, images 1200 and 1270 may be a bright-field image depicting a sample container or well storing iPSCs, and images 1250 and 1290 may depict the generated mask representation overlaid on images 1200 and 1270, respectively. Confluence score determination subsystem 134 may be configured to determine whether the computed confluence score for a sample container or well of sample containers or wells 304 satisfies a confluence threshold condition. For example, the confluence threshold condition may be satisfied if the confluence score falls within a predefined range of confluence scores (e.g., a confluence score between 65% and 85% confluence).

In some embodiments, confluence score determination subsystem 134 may use one or more machine learning models to compute a confluence score for each of sample containers or wells 304. For example, a confluence model may be trained to compute a confluence of iPSCs within a sample container or well based on an image of the sample container or well. The confluence model may be trained to generate a mask representation for an image and determine the confluence score based on the mask representation. For example, a percentage of pixels representing portions of the image depicting one or more alive iPSCs may be computed. The confluence score may be based on the percentage. For example, with reference to FIGS. 12A-12B, if the mask representation of image 1250 is determined to have 60% pixels with one or more alive iPSCs, then the confluence score for bright-field image 1200 may be 60%. In some embodiments, the confluence score may be a continuous value (e.g., a numerical value between 0-100). Alternatively, or additionally, confluence score determination subsystem 134 may generate a discrete confluence score (e.g., classifying each sample container into one of a set of confluence classifications). For example, the one or more machine learning models may include a convolutional neural network (CNN) trained to analyze images 342 and output a confluence score for each of sample containers or wells 304, and the confluence scores may be passed to a classifier trained to classify each of sample containers or wells 304 into one of a set of confluence classifications based on the confluence score of that sample container or well. For example, the CNN may analyze images 342 and generate a mask representation for each of images 342. The mask representations may be used to determine a confluence score for each of images 342. For example, the mask representations may depict pixels representing one or more alive iPSCs in one manner and pixels representing other contents (e.g., one or more dead iPSCs, debris, condensation, cell media not containing iPSCs, etc.) in another manner. The confluence score may be determined based on a percentage of the mask representation composed of pixels in the first manner.

The set of confluence classifications may include a first confluence classification and a second confluence classification. A sample container assigned the first confluence classification may correspond to the sample container's confluence score being within the predefined range of confluence scores. In some embodiments, a confluence score within the predefined range of confluence scores may correspond to a confluence score that is greater than or equal to a first threshold confluence score (e.g., 65% or more occupied) and less than or equal to a second threshold confluence score (e.g., 85% or less occupied). For example, the first confluence classification may be assigned to sample containers having a confluence score that is between 65-85% occupied range. A sample container or well assigned the second confluence classification may correspond to a sample container or well having a confluence score that is not within the predefined range of confluence scores. For example, the second confluence classification may be assigned to sample containers or wells having a confluence score that is less than the first threshold confluence score or greater than the second threshold confluence score.

As mentioned previously, computing system 130 may include sample container selection subsystem 136. Sample container selection subsystem 136 may be configured to generate and output an instruction 344 based on the analyses performed by quality score determination subsystem 132 and/or confluence score determination subsystem 134 and optionally a growth metric for each sample. Instruction 344 may indicate an action to be performed to the iPSCs stored within some or all of sample containers or wells 304 based on the quality score and/or the confluence score. In some embodiments, sample container selection subsystem 136 may be configured to rank sample containers 304 based on a quality score and/or a confluence score computed for that sample container or well. Sample container selection subsystem 136 may obtain a quality score and a confluence score for each sample container and may generate a ranking of the sample containers or wells based on the quality score and confluence score. The quality score and the confluence score in this example may be continuous scores. Therefore, sample container selection subsystem 136 may aggregate, average, or otherwise combine the quality score and the confluence score of a given sample container and rank that sample container or well with respect to the other sample containers or wells. The top N sample containers or wells may be selected by sample container selection subsystem 136 for passaging of the iPSCs stored therein.

Sample container selection subsystem 136 may generate instruction 344 to indicate the action to be performed to/with a subset of iPSCs 312 stored in each of sample containers or wells 304. In some embodiments, instruction 344 may indicate that a subset of iPSCs 312 stored in one or more of sample containers or wells 304 is to be discarded, fed, or passaged based on the quality score and/or confluence score determined for those sample containers or wells 304. Sample container selection subsystem 136 may generate instruction 344 may be based on the quality score and/or confluence score of each of sample containers or wells 304.

In some embodiments, sample container selection subsystem 136 may determine that a subset of iPSCs 312 stored in one or more of sample containers or wells 304 should be discarded based on the quality classification of sample containers or wells 304. For example, if a sample container or well is assigned the empty classification or the low-quality classification, the subset of iPSCs 312 stored in that sample container or well may be discarded. Sample container selection subsystem 136 may generate instruction 344 indicating the sample container or sample containers that have been assigned the empty classification and/or low-quality classification. Instruction 344 may be provided to cell handling system 300. Cell handling system 300 may select the sample containers or wells assigned the empty classification and/or low-quality classification and may provide the selected sample containers or wells to cell discard system 360. Cell discard system 360 may be configured to discard the subset of iPSCs 312 stored within the sample containers assigned the empty classification and/or low-quality classification.

In some embodiments, sample container selection subsystem 136 may determine that a subset of iPSCs 312 stored in one or more of sample containers or wells 304 are to be fed to increase the quality and/or quantity of the iPSCs stored within those sample containers or wells.

For example, if a sample container or well is assigned the low-quality classification or the medium-quality classification, the subset of iPSCs 312 stored within that sample container or well may be fed. Sample container selection subsystem 136 may generate instruction 344 indicating the sample container or well, or sample containers or wells, that have been assigned the low-quality classification or the medium-quality classification. Instruction 344 may be provided to cell handling system 300. Cell handling system 300 may select the sample containers assigned the low-quality classification or medium-quality classification and may provide the selected sample containers or wells to cell feeding system 330. Cell feeding system 330 may be configured to perform one or more additional feedings to the subset of iPSCs 312 stored within the sample containers assigned the low-quality classification or the medium-quality classification.

In some embodiments, sample container selection subsystem 136 may determine that passaging is to be performed on a subset of iPSCs 312 stored in one or more of sample containers 304 based on the quality classification of sample containers 304. For example, if a sample container is assigned the high-quality classification, the subset of iPSCs 312 stored within that sample container may be selected for passaging. Sample container selection subsystem 136 may generate instruction 344 indicating the sample container or sample containers that have been assigned the high-quality classification. Instruction 344 may be provided to cell handling system 300. Cell handling system 300 may select the sample containers or wells assigned the high-quality classification and may provide the selected sample containers or wells to passaging system 350. Passaging system 350, as described below, may be configured to perform passaging of the subset of iPSCs 312 stored within the selected sample containers or wells.

In some embodiments, sample container selection subsystem 136 may determine that passaging is to be performed on a subset of iPSCs 312 stored in one or more of sample containers or wells 304 based on the confluence classification of sample containers or wells 304. For example, if a sample container or well is assigned the first confluence classification (e.g., the confluence score of that sample container is within a predefined range of confluence scores), the subset of iPSCs 312 stored within that sample container or well may be selected for passaging. Sample container selection subsystem 136 may generate instruction 344 indicating the sample container or well, or sample containers or wells, that have been assigned the first confluence classification. Instruction 344 may be provided to cell handling system 300. Cell handling system 300 may be configured to select the sample containers or wells assigned the first confluence classification and may provide the iPSCs stored in the selected sample containers or well to passaging system 350. Passaging system 350, as described below, may be configured to perform passaging of the subset of iPSCs 312 stored within the selected sample containers or wells.

In some embodiments, sample container selection subsystem 136 may determine that a subset of iPSCs 312 stored in one or more of sample containers or wells 304 are to be fed to increase the confluence of the iPSCs stored within those sample containers. For example, if a sample container or well is assigned the second confluence classification (e.g., the confluence score of that sample container is not within a predefined range of confluence scores), the subset of iPSCs 312 stored within that sample container or well may be fed. Sample container selection subsystem 136 may generate instruction 344 indicating the sample container or well, or sample containers or wells, that have been assigned the second confluence classification. Instruction 344 may be provided to cell handling system 300. Cell handling system 300 may be configured to select the sample containers assigned the second confluence classification and may provide the iPSCs stored in the selected sample containers or wells to cell feeding system 330. Cell feeding system 330 may be configured to perform one or more additional feedings to the subset of iPSCs 312 stored within the sample containers or wells assigned the second confluence classification.

In some embodiments, cell handling system 300 may be configured to select at least one of the sample containers or wells 304 based on instruction 344. The selected sample container or well may be provided to passaging system 350. Passaging may be performed after a predefined amount of time has elapsed from the thawing of iPSCs 312. Passaging system 350 may be configured to perform passaging of a subset of iPSCs 312 stored in each selected sample container or well. In some embodiments, passaging may include washing the subset of iPSCs 312 stored in each selected sample container or well with a cell dissociation reagent. The cell dissociation reagent may function to cause iPSCs within the selected sample container to detach from the sample container's walls. Passaging may also include incubating the washed subset of iPSCs 312 stored in the selected sample container or well with the cell dissociation reagent, optionally using an on-deck incubator. Passaging may also include triturating the incubated subset of iPSCs 312 after a media is added to the incubated subset of iPSCs. For example, a stabilized feeder-free maintenance medium containing a selective small molecule inhibitor may be added to the incubated subset of iPSCs after a predefined amount of time has elapsed (e.g., approximately 10 minutes). Passaging may also include transferring the triturated subset of iPSCs 312 to a sample container block. In one embodiment, the sample container block, for example, may be a 96-well deep well plate block. Passaging may also include centrifuging the transferred subset of iPSCs 312 in the sample container block to pellet the centrifuged subset of iPSCs 312. Passaging may further include performing a buffering exchange to the pelleted subset of iPSCs by aspirating the pelleted subset of iPSCs 312 (e.g., aspirating the stabilized feeder-free maintenance medium containing the selective small molecule inhibitor and the cell dissociation reagent). Passaging may further include suspending (or re-suspending) the aspirated subset of iPSCs 312 into the media. In particular, the suspending of the subset of iPSCs 312 may include suspending the iPSCs in fresh stabilized feeder-free maintenance medium containing the selective small molecule inhibitor. Persons of ordinary skill in the art will recognize that modifications to the steps may occur, and the aforementioned is an example of the passaging process. In some embodiments, passaging system 350 and/or cell handling system 300 may be configured to count the passaged iPSCs and distribute the passaged iPSCs or a subset of the passaged iPSCs into a second plurality of sample containers or wells. For example, the passaged iPSCs or a subset thereof may be distributed across sample containers or wells 354a-354 (collectively “sample containers or wells 354”) of multi-sample or multi-well plate 352. In some embodiments, passaging system 350 and/or cell handling system 300 may use a cell counter to count out a predetermined quantity of iPSCs (e.g., 50×10⁴ iPSCs, 100×10⁴ iPSCs, 150×10⁴ iPSCs, 200×10⁴ iPSCs, or the like). The predetermined quantity of iPSCs may be plated into sample containers or wells 354 of multi-sample or multi-well plate 352. Multi-sample or multi-well plate 352 may include sample containers or wells 354, which may be pre-coated with a recombinant human protein and pre-filled with stabilized feeder-free maintenance medium containing a selective small molecule inhibitor.

In some embodiments, passaging system 350 may be configured to perform one or more rounds of passaging of the subset of iPSCs 312 stored in the selected sample containers. Passaging system 350 may determine whether to perform additional rounds of passaging based on an amount of time that has elapsed since iPSCs 312 were thawed. For example, passaging system 350 may perform passaging daily until a predefined amount of time has elapsed (e.g., 5 days). In some embodiments, if passaging system 350 determines that the amount of time that has elapsed since iPSCs 312 were thawed is less than the predefined amount of time, then the subset(s) of iPSCs 312 stored in the selected sample containers (that have been passaged already) may be provided to cell feeding system 330 via cell handling system 300. Cell feeding system 330 may be configured to perform one or more additional feedings to these iPSCs, and the imaging and analysis of these iPSCs may be repeated until the predefined amount of time has elapsed. The multiple rounds of passaging may be performed to ensure that a threshold quantity of iPSCs are within the selected sample containers. For example, each of sample containers 354 may include approximately 1.5×10⁶ iPSCs. Passaging system 350 may be configured to provide the passaged iPSCs stored in sample containers 354 to differentiation processing system 120 based on a determination that passaging has completed.

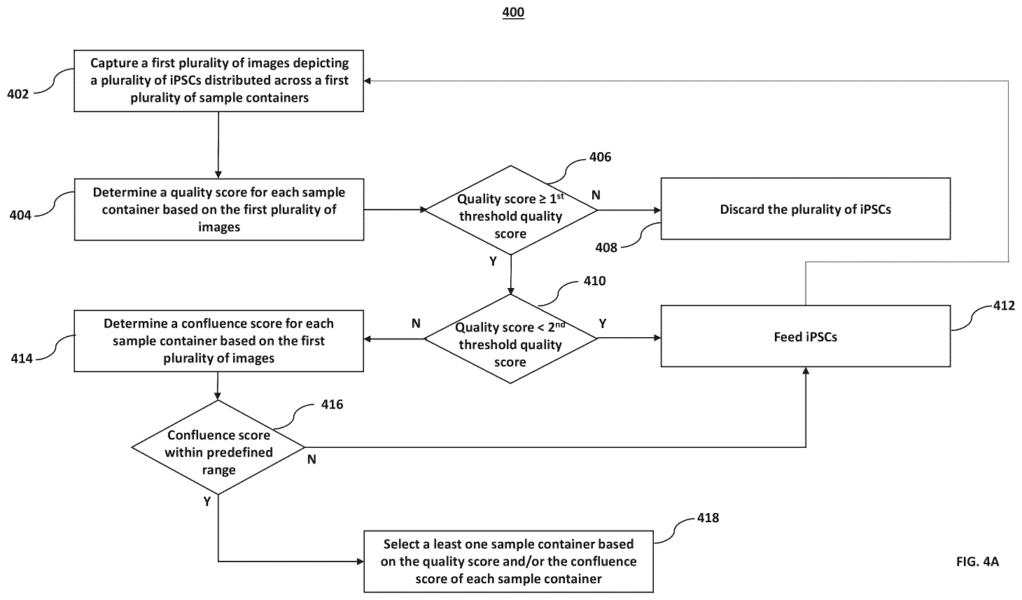

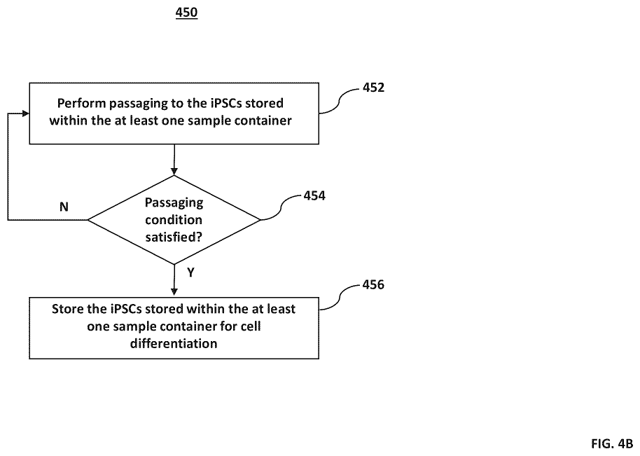

FIG. 4A illustrates an exemplary method 400 for selecting, based on a maintenance process performed on iPSCs, at least one sample container of iPSCs for passaging, in accordance with some embodiments. Method 400 is performed, for example, using one or more electronic devices implementing a software platform, such as AMD platform 100. In some examples, method 400 is performed using a client-server system, and the steps of method 400 are divided up in any manner between the server (e.g., AMD platform 100) and one or more client devices (e.g., client device 102). Thus, while portions of method 400 are described herein as being performed by particular devices of a client-server system, it will be appreciated that method 400 is not so limited. In other examples, method 400 is performed using only a client device or only multiple client devices. In method 400, some blocks are, optionally, combined, the order of some blocks is, optionally, changed, and some blocks are, optionally, omitted. In some examples, additional steps may be performed in combination with method 400. Accordingly, the operations as illustrated (and described in greater detail below) are exemplary by nature and, as such, should not be viewed as limiting.

Method 400 of FIG. 4A may, in some embodiments, begin at operation 402. In operation 402, a first plurality of images depicting a plurality of iPSCs distributed across a first plurality of sample containers may be captured. In some embodiments, the first plurality of images may depict some or all of the first plurality of sample containers or wells. In some embodiments, the first plurality of images may be captured using an imaging system. For example, images 342 of sample containers or wells 304 may be captured using imaging system 340 of maintenance processing system 110. In some embodiments, computing system 130 may generate instructions 344 to cause imaging system 340 to capture images 342 of sample containers or wells 304. In some embodiments, images 342 may depict iPSCs stored in sample containers or wells 304 after the iPSCs have been thawed, distributed, and fed.

In operation 404, a quality score for each sample container or well may be determined based on the first plurality of images. For example, a quality score may be computed for each of sample containers or wells 304. The quality score may indicate a quality of the iPSCs stored within each of sample containers or wells 304. In some embodiments, a quality score classification for each sample container or well 304 may be determined based on the computed quality score. In some embodiments, one or more machine learning models may be provided with the first plurality of images. The machine learning models may be trained to output a quality score representing a quality of the iPSCs stored within each sample container depicted by the first plurality of images.

In operation 406, a determination may be made as to whether the quality score of any of sample containers or wells 304 is greater than or equal to a first threshold quality score. In some embodiments, the first threshold quality score may serve as a bar to determine whether the iPSCs stored in any of sample containers or wells 304 should be discarded. For example, sample containers that are classified as being “empty” may have a quality score that is less than the first threshold quality score. Empty sample containers or wells may be discarded as there may not be enough iPSCs stored therein for passaging and/or cell differentiation. Some example values for the first threshold quality score may be a score of 20/100 or less, 15/100 or less, 10/100 or less, or other values. Alternatively, threshold quality scores may be labels rather than numeric quality scores.

If, at operation 406, it is determined that the quality score of one or more of sample containers or wells 304 is less than the first threshold quality score, then method 400 may proceed to operation 408. In operation 408, iPSCs stored within the one or more sample containers having a quality score less than the first threshold quality score may be discarded.

If, however, at operation 406, it is determined that the quality score of one or more of sample containers or wells 304 is greater than or equal to the first threshold quality score, then method 400 may proceed to operation 410.

In operation 410, a determination may be made as to whether the quality score of the one or more sample containers or wells 304 is less than a second threshold quality score. In some embodiments, the second threshold quality score may serve as a bar to determine whether the iPSCs stored in sample containers or well 304 should be passaged. For example, sample containers or wells that are classified as being “low-quality” or “medium-quality” may have a quality score that is greater than or equal to the first threshold quality score but is also less than the second threshold quality score. Some example values for the first threshold quality score may be a score of 70/100 or less, 60/100 or less, 50/100 or less, or other values. Alternatively, threshold quality scores may be labels rather than numeric quality scores.

If, at operation 410, it is determined that the quality score of one or more of sample containers or wells 304 is less than the second threshold quality score and also greater than or equal to the first threshold quality score, then method 400 may proceed to operation 412. In operation 412, the iPSCs stored within the one or more of sample containers or wells 304 may be fed. After the iPSCs stored within the one or more sample containers or wells 304 have been fed in operation 412, method 400 may return to operation 402. Here, new images of the sample containers may be captured and the quality score for these re-fed sample containers storing iPSCs may be determined.

In some embodiments, after the iPSCs stored in the one or more of sample containers or wells 304 have been fed in operation 412, a second plurality of images depicting the iPSC stored in sample containers or wells 304 may be captured. For example, sample containers or wells 304 may be fed again by cell feeding system 330, and then imaged using imaging system 340. In some embodiments, an updated quality score and/or updated confluence score may be determined for each of the one or more of sample containers or wells 304 using the machine learning models. For example, the second plurality of images may be provided to computing system 130. Quality score determination subsystem 132 and confluence score determination subsystem 134 may respectively compute an updated quality score for sample containers or wells 304 based on the second plurality of images. In some embodiments, at least one of the one or more sample containers or wells may be selected based on the updated quality score and/or the updated confluence score of the one or more sample containers or wells. For example, quality score determination subsystem 132 may determine whether the updated quality scores satisfy a quality threshold condition and confluence score determination subsystem 134 may determine whether the updated confluence scores satisfy a confluence threshold condition.

If, however, at operation 410 it is determined that the quality score of the one or more of sample containers or wells 304 is greater than or equal to the second threshold quality score, then method 400 may proceed to operation 414. In operation 404, a confluence score for some or all of sample containers or wells 304 may be determined. In some embodiments, one or more machine learning models may be provided with the first plurality of images. The machine learning models may be trained to output a confluence score representing a confluence of iPSCs stored within the sample containers depicted by the first plurality of images.

In some embodiments, a confluence score may be determined for the one or more of sample containers or wells 304 determined to have a quality score that is greater than or equal to the first threshold quality score and the second threshold quality score. In some embodiments, a confluence score may be determined for each of sample containers or wells 304. In this case, the images captured during operation 402 may also be analyzed to determine the confluence score of the sample containers depicted therein, and, subsequent to the analysis of the confluence score, the images may be analyzed to determine a quality score of those sample containers or wells. In other words, while method 400 is illustrated to depict images being captured and analyzed for quality followed by a confluence analysis, alternatively, the images may be analyzed for confluence followed by a quality analysis. Still further, some embodiments include operations 404 and 414 being performed substantially in parallel.

In operation 416, a determination may be made as to whether the confluence score for sample containers or wells 304 is within a predefined range of confluence scores. The predefined range of confluence scores may include confluence scores that are greater than or equal to a first threshold confluence score (e.g., 65% confluence score) and less than a second threshold confluence score (e.g., 85% confluence score). Some additional example threshold ranges include threshold ranges from about 20% to about 90%, about 25% to about 85%, about 30% to about 80%, or other ranges. Some additional example threshold ranges include about 50% to about 80%, from about 60% to about 80%, and/or from about 65% to about 75%. Alternatively, threshold ranges may be labels rather than numeric quality scores.

If, in operation 416, it is determined that the confluence score for one or more of sample containers or wells 304 is not within the predefined range of confluence scores, then method 400 may proceed to operation 412 where the iPSCs stored within the one or more of sample containers or wells 304 may be fed.

If, in operation 416, it is determined that the confluence score of the one or more of sample containers or wells 304 is within the predefined range of confluence scores, then method 400 may proceed to operation 418. In operation 418, at least one sample container or well 304 may be selected based on the quality score and the confluence score. The at least one selected sample container or well may be provided to a passaging system (e.g., passaging system 350) such that the iPSCs stored therein may be passaged. In some embodiments, each sample container determined to satisfy the quality threshold condition (e.g., quality score greater than or equal to a first threshold quality score and greater than equal to a second threshold quality score) may be selected. In some embodiments, only some of the sample containers or wells determined to satisfy the quality threshold condition (e.g., quality score greater than or equal to a first threshold quality score and greater than equal to a second threshold quality score) may be selected. In some embodiments, a ranking of the sample containers or wells may be determined based on quality score and/or the confluence score of each of the one or more sample containers or wells. Then at least one sample container or well may be selected based on the ranking.

In some embodiments, the one or more machine learning models may include a first machine learning model and a second machine learning model. The first machine learning model may be trained to output a quality score indicating a quality of the iPSCs stored within a sample container based on images depicting the sample container. The second machine learning model may be trained to output a confluence score indicating a confluence of iPSCs stored within a sample container based on images depicting the sample container. The first machine learning model and/or the second machine learning model may be a computer vision model. For example, the computer vision model may be implemented using a convolutional neural network (CNN) framework. The first and second machine learning models may be based on the same or different architectures. For example, the first machine learning model may be built on a ResNet architecture (e.g., ResNet-18) and the second machine learning may be built on a U-Net architecture. In some embodiments, a single machine learning model may be trained to determine a quality score and a confluence score for the sample containers based on images depicting the sample containers.

FIG. 4B illustrates an exemplary method 450 for performing passaging of iPSCs stored within one or more selected sample containers or wells, in accordance with some embodiments. Method 450 is performed, for example, using one or more electronic devices implementing a software platform, such as AMD platform 100. In some examples, method 450 is performed using a client-server system, and the steps of method 450 are divided up in any manner between the server (e.g., AMD platform 100) and one or more client devices (e.g., client device 102). Thus, while portions of method 450 are described herein as being performed by particular devices of a client-server system, it will be appreciated that method 450 is not so limited. In other examples, method 450 is performed using only a client device or only multiple client devices. In method 450, some blocks are, optionally, combined, the order of some blocks is, optionally, changed, and some blocks are, optionally, omitted. In some examples, additional steps may be performed in combination with method 450. Accordingly, the operations as illustrated (and described in greater detail below) are exemplary by nature and, as such, should not be viewed as limiting.

In some embodiments, method 450 may be an extension of method 400. For example, method 450 may begin after method 400 has ended, as described in greater detail below.

Method 450 of FIG. 4B may, in some embodiments, begin at operation 452. In operation 452, passaging may be performed to the iPSCs stored within the at least one selected sample container or well. The at least one selected sample container or well may correspond to the sample container(s) or well(s) selected in operation 418 of method 400 shown in FIG. 4A. In some embodiments, passaging may include one or more of: washing a subset of iPSCs stored within the at least one selected sample container or well, incubating the washed subset of iPSCs with a dissociation reagent, triturating the incubated subset of iPSCs after a media is added to the incubated subset of iPSCs, transferring the triturated subset of iPSCs to a sample container block, centrifuging the transferred subset of iPSCs in the sample container block to pellet the centrifuged subset of iPSCs, performing a buffering exchange to the pelleted subset of iPSCs by aspirating the pelleted subset of iPSCs, and suspending the aspirated subset of iPSCs into the media.

In operation 454, a determination may be made as to whether a passaging condition has been satisfied. Passaging refers to a procedure for harvesting iPSCs from a culture, transferring the iPSCs to one or more culture vessels with a fresh growth medium, and using those iPSCs to start new cultures. Passaging may ensure that the iPSCs within a sample container or well maintain a particular level of quality and confluence so that those iPSCs may be subjected to cell differentiation. Passaging of the iPSCs may occur after a predefined amount of time has elapsed since thawing. In some embodiments, multiple rounds of passaging may be performed before subjecting the cells to one or more cell differentiation steps. For example, two rounds of passaging may be performed to the iPSCs stored within the selected sample containers prior to cell differentiation. In some embodiments, the cell differentiation steps may be optional. In some embodiments, the passaging condition being satisfied includes a predefined amount of time since the iPSCs were thawed elapsing.

If, in operation 454, it is determined that the passaging condition has not been satisfied, then method 400 may return to operation 452. In operation 452, additional rounds of passaging may be performed to the iPSCs stored in the at least one sample container or well that was selected.

If, however, in operation 454, it is determined that the passaging condition has been satisfied, then method 400 may proceed to operation 456. In operation 456, the passaged iPSCs may be stored for cell differentiation. For example, the passaged iPSCs may be frozen for subsequent subjection to one or more cell differentiation steps.

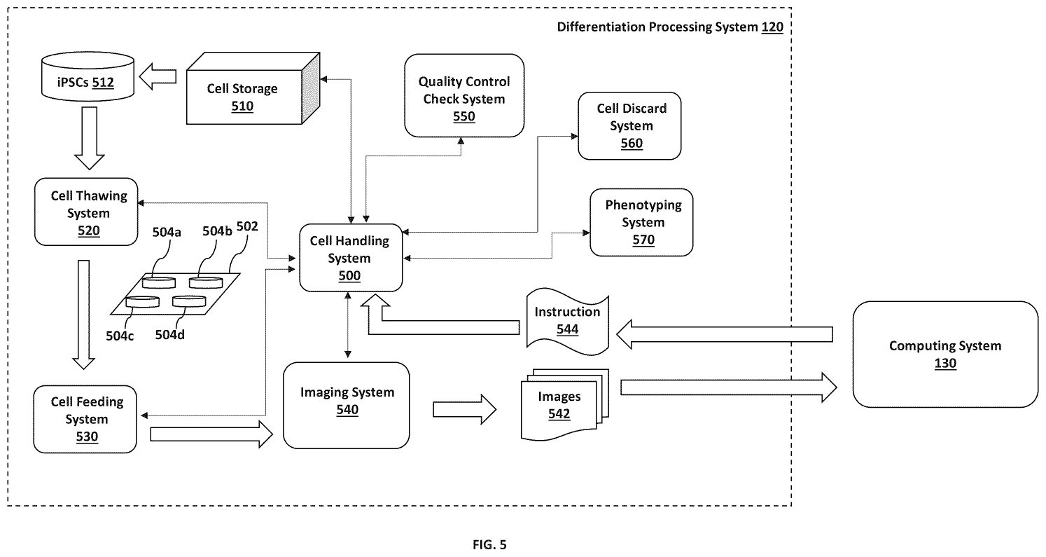

FIG. 5 illustrates an example differentiation processing system 120 for performing cell differentiations, in accordance with some embodiments. Differentiation processing system 120 may include a cell handling system 500, a cell storage 510, a cell thawing system 520, a cell feeding system 530, an imaging system 540, a quality control (QC) check system 550, a cell discard system 560, or other components. Differentiation processing system 120 may communicate with computing system 130 or other components of system 10 (shown in FIG. 1A) to perform one or more evaluations and/or facilitate one or more actions relating to the process of differentiating iPSCs (e.g., optionally subjecting iPSCs to cell differentiation steps, performing quality control checks, phenotyping, etc.). In some embodiments, cell handling system 500, cell storage 510, cell thawing system 520, cell feeding system 530, imaging system 540, and cell discard system 560 may be the same or similar to cell handling system 300, cell storage 310, cell thawing system 320, cell feeding system 330, imaging system 340, and cell discard system 360 of maintenance processing system 110, and the previous description may apply.

Cell handling system 500 may be configured to transport iPSCs to various components of differentiation processing system 120. Cell handling system 500 may include one or more robotic devices that receive instructions from computing system 130 or other components of system 10 (e.g., client device 102 of FIG. 1A). Instructions 544 for controlling the movements of the robotic devices of cell handling system 500 may be generated by computing system 130. The instructions may cause the robotic devices of cell handling system 500 to perform various actions, such as, but not limited to, retrieving iPSCs 512 from cell storage 510, providing iPSCs 512 to cell thawing system 520, distributing iPSCs 512 across a plurality of sample containers 504a-504d of a multi-sample plate 502 (e.g., across a plurality of sample wells of a multi-well plate), providing multi-sample plate 502 to cell feeding system 530 for feeding iPSCs 512, providing multi-sample plate 502 to imaging system 540 for capturing images of iPSCs 512 stored in sample containers 504a-504b (collectively “sample containers 504”), selecting a subset or subsets of iPSCs 512 stored in sample containers 504, performing one or more cell differentiation steps to a subset or subsets of iPSCs 512 stored in sample containers 504, performing a quality control (QC) check or checks to a subset or subsets of iPSCs 512 stored in sample containers 504, performing one or more phenotypic assessments to the subset or subsets of iPSCs 512 stored in sample containers 504, discarding one or more subsets of iPSCs 512, or to have other actions performed to iPSCs 512.

Although a single cell handling system 500 is depicted in differentiation processing system 120, it should be understood that multiple robotic devices may be implemented by cell handling system 500. These robotic devices may perform various actions using the components of differentiation processing system 120. For example, cell handling system 500 may include a robotic device configured to retrieve iPSCs 512 from cell storage 510 and distribute iPSCs 512 across sample containers 504, a robotic device configured to transport sample containers 504 to cell thawing system 520, etc. Cell handling system 500 may also perform tasks conventionally performed by human operators.

Cell storage 510 may be configured to store iPSCs, including iPSCs 512, to be developed into differentiated cells. In some embodiments, cell storage 510 may store the passaged iPSCs 512. Cell storage 510 may be configured to store iPSCs at a particular temperature to preserve the iPSCs for future use. For example, cell storage 510 may operate at a temperature of −195.8° C., however other temperatures may be used. Within cell storage 510, the batches of iPSCs may be stored in one or more vials designed to safely store and track iPSCs.

In some embodiments, cell handling system 500 may retrieve iPSCs 512 from cell storage 310. An instruction 544 may be generated by computing system 130 and provided to cell handling system 500 indicating which iPSCs 512 to retrieve. In some embodiments, instruction 544 may be generated by computing system 130 based on a command provided by client device 102. For example, a user may indicate a batch or batches of iPSCs 512 to retrieve from cell storage 510. Instruction 544 may cause cell handling system 500 to retrieve vials storing iPSCs 512 from cell storage 310. In some embodiments, a user may select which iPSCs 512 to retrieve from cell storage 310. The user may input a command to AMD platform 100 using client device 102. The command may specify the iPSCs to retrieve from cell storage 510 (e.g., iPSCs 512). For example, the command may cause a robotic device of cell handling system 300 to physically retrieve iPSCs 512 from cell storage 510. In some embodiments, iPSCs 512 may include 100 or more iPSCs, 1,000 or more iPSCs, 10,000 or more iPSCs, 100,000 or more iPSCs, 1,000,000 or more iPSCs, 10,000,000 or more iPSCs, or other quantities.

Cell handling system 500 may provide iPSCs 512, retrieved from cell storage 510, to cell thawing system 520. Cell thawing system 520 may be configured to seed iPSCs 512 into a second plurality of sample containers 504a-504d (collectively “sample containers 504”) of multi-sample plate 502 (e.g., a second plurality of sample wells of a multi-well plate). As an example, a sample container may be seeded at a particular same density (e.g., 300,000 iPSCs in a twelve-sample container). Cell thawing system 520 may be configured to thaw iPSCs 512. In some embodiments, cell thawing system 520 may thaw iPSCs 512 in a same or similar manner as that of cell thawing system 320, and the previous description may apply.

Cell handling system 500 may be configured to provide multi-sample plate 502, which may include iPSCs 512 distributed across sample containers 504, to cell feeding system 530. Cell feeding system 530 may be configured to perform a first cell differentiation step by performing one or more feedings to each subset of iPSCs 512 stored within sample containers 504. To feed each subset of iPSCs 512, cell feeding system 530 may be configured to use a media that induces differentiation of iPSCs 512. For example, a cell-type specific media may be selected and used to induce differentiation towards a particular type of cell (e.g., motor neuron). Exemplary media may include, but is not limited to, BrainPhys™ Neuronal Medium, MEM Non-Essential Amino Acids (NEAA), GlutaMAX™, N2 Supplement (100×), B-27 Plus Supplement (50×), BDNF Recombinant Human BDNF protein, rhNT-3, Doxycycline, or other media. In some embodiments, cell feeding system 530 may perform a feeding at a particular cadence. For example, feeding may be performed every 24 hours. During the times when the iPSCs are not fed, the iPSCs may be incubated by cell feeding system 530 at a temperature of 37° C.

After cell feeding system 530 performs the feedings, cell handling system 500 may provide multi-sample plate 502 to imaging system 540. In some embodiments, imaging system 540 may be a digital microscopy imaging system configured to capture digital images (e.g., whole slide images). In some embodiments, the images may be bright-field, phase contrast, or fluorescent images. Imaging system 540 may be configured to capture a plurality of images depicting sample containers 504 storing each subset of iPSCs 512 subsequent to cell feeding system 530 performing the first cell differentiation step. In some embodiments, images 542 may be whole slide images depicting some or all of sample containers 504. For example, the whole slide images may be 100,000×100,000 pixels. As another example, images 542 may each be JPG files of 1,224×904 pixels. In some embodiments, images 542 of sample containers 504 may be captured at different magnification levels (e.g., 5× magnification, 10× magnification, 20× magnification, etc.). In some embodiments, each of images 342 may be divided into a plurality of tiles. The tiles may be of a smaller size (e.g., 512×512 pixels), which may facilitate faster computations. A number of images 542 and a resolution, magnification, etc., may vary and may be determined based on instructions 544 provided to imaging system 540 from computing system 130. In some embodiments, 4×4 (16 field of views total) may be captured at a particular magnification level (e.g., 4× magnification, 10× magnification, etc.). For example, imaging system 540 may capture bright-field images at 14× magnification.

In some embodiments, cell handling system 500 may determine whether a first predefined amount of time has elapsed from the feeding of the distributed subsets of iPSCs 512 in each of sample containers 504. If the first predefined amount of time has not elapsed, cell handling system 500 may return iPSCs to cell feeding system 530 to perform another cell differentiation step to iPSCs 512. However, if the first predefined amount of time has elapsed, cell handling system 500 may be configured to bank iPSCs 512 stored in sample containers 504 in cell storage 510. iPSCs 512 stored in the banked sample containers may be referred to as a “lot” of iPSCs. In some embodiments, the first predefined amount of time may be three days, however other amounts of time may be used (e.g., 1 or more days, 5 or more days, 10 or more days, etc.). The amount of time may be selected such that progenitors can sufficiently expand. The sufficient expansion of the progenitors may enable the banked iPSCs to be frozen without the iPSCs becoming too differentiated for survival following the freezing and replating during subsequent differentiation steps. Banking the lot of iPSCs may include freezing the iPSCs at a temperature that inhibits cell growth and cell death.

In some embodiments, computing system 130 may be configured to assess a quality of a differentiation as it proceeds. The machine learning model may be stored in model database 152. This machine learning model may be cell type specific. As an example, to train a machine learning model to assess the differentiation for motor neurons, a number of motor neuron differentiations may be performed to obtain training data describing failed and successful differentiations. The training data may be stored in training data database 154. The training data may include the failed/successful differentiation information matched with QPC imaging or bright-field imaging. The training data may be used to train a machine learning model to predict whether a differentiation step will be successful as the differentiation is occurring.

In some embodiments, quality control (QC) check system 550 may be configured to perform a QC check to the lot of iPSCs stored in the banked sample containers. In some embodiments, imaging system 540 may provide images 542 to computing system 130 for analysis. For example, quality control (QC) check subsystem 142 may be configured to perform a QC check to one or more subsets of iPSCs 512. For example, QC check subsystem 142 may select a sample container from the second plurality of sample containers 504. A quality control (QC) check may be performed to the iPSCs stored within the selected sample container of the second plurality of sample containers (e.g., sample containers 504). The QC check may serve as a proxy for the lot of iPSCs banked in cell storage 510.

In some embodiments, QC check system 550 may be configured to perform the QC check to iPSCs stored within the selected sample container of the second plurality of sample containers. QC check system 550 may be configured to stain the iPSCs. iPSCs that have been stained cannot be differentiated. Therefore, to prevent damaging the lot of iPSCs, the iPSCs from one sample container may be selected by cell handling system 500 and/or computing system 130. For example, instructions 544 may indicate which sample container 504 is to be selected for the QC check. If these iPSCs pass the QC check, then the lot of iPSCs banked in cell storage 510 may be released for further cell differentiation. However, if these iPSCs do not pass the QC check, cell handling system 500 may provide some or all of the lot of iPSCs to cell discard system 560. Cell discard system 560 may be configured to discard the lot. In some embodiments, the sample container may be randomly selected from the second plurality of sample containers for performing the QC check.

QC check system 550 may be configured to instruct cell thawing system 520 and cell feeding system 530 to thaw and feed the iPSCs stored in the selected sample container. In some embodiments, QC check system 550 may cause the cell handling system to retrieve the iPSCs stored in the selected sample container from cell storage 510. The retrieved iPSCs may then be thawed by cell thawing system 520 and subsequently fed by cell feeding system 530. Thawing the iPSCs may include placing (e.g., via one or more robotic devices controlled by cell handling system 500) vials of the iPSCs in a water bath (e.g., at 37° C.). Cell handling system 500 may transfer the contents of the vial to a block of sample containers, and the vial may be washed with a cell-type specific media. The block of sample containers may be centrifuged in the liquid in the block and then aspirated. The cell-type specific media may then be added to resuspend the iPSCs.

In some embodiments, a cell count may be performed. The cell count may be performed manually and/or using one or more machine learning models. For example, a computer vision model trained to detect iPSCs may be used to estimate a quantity of iPSCs that have been resuspended. Images depicting the selected sample containers after thawing and feeding may be captured using imaging system 540. These images (e.g., images 542) may be analyzed by the machine learning models of computing system 130 to determine the cell count. Based on the estimated quantity of iPSCs, QC check system 550 may cause a concentration of the iPSCs and media to be adjusted to meet requirements for plating/seeding. The iPSCs (e.g., the iPSCs from the selected sample container of the second plurality of sample containers that has been resuspended) may then be seeded in a third plurality of sample containers of a multi-sample plate.

In some embodiments, computing system 130 may instruct (e.g., via instructions 544) cell feeding system 530 to feed the iPSCs stored in the third plurality of sample containers. These iPSCs may be fed at a predefined frequency for cell differentiation. For example, the iPSCs may be fed every two days up until a second threshold amount of time has elapsed (e.g., 10 days). In some embodiments, the iPSCs may be fed using 80 micro-liters of media aspirated from the sample containers. Furthermore, 100 micro-liters of fresh media may be dispensed into the sample containers.

In some embodiments, computing system 130 may instruct (e.g., via instructions 544) imaging system 540 to capture a third plurality of images of the iPSCs stored in the third plurality of sample containers. Analyzing the third plurality of images to determine that the iPSCs may ensure that the iPSCs are developing as expected. For example, these images may be used for routine inspection to ensure that seeding is uniform, and that the iPSCs stored in the sample containers have a target cell density/confluence. Furthermore, these images may be used to ensure that no contaminants are present in the third plurality of sample containers.

In some embodiments, computing system 130 and/or QC check system 550 may be configured to determine whether a second predefined amount of time has elapsed from the feeding of the iPSCs stored within the selected sample container. As an example, the second predefined amount of time may be 10 days, however other amounts of time may be used. 10 days may represent a typical amount of time that iPSCs need to be fed at a particular cadence in order to obtain differentiated cells that can be analyzed using biomarkers for quality. If computing system 130 and/or QC check system 550 determine that the second predefined amount of time has not elapsed, then cell handling system 500 may be instructed (e.g., via instructions 544) to transport the iPSCs back to cell feeding system 530 to repeat the feeding. After the feeding has been repeated, cell handling system 500 may be instructed (e.g., via instructions 544) to provide the iPSCs to imaging system 540 to capture additional images of the iPSCs stored within the sample container selected for the QC check. For example, an additional round of feeding may be performed to the iPSCs stored in the third plurality of sample containers, after which imaging and a check of whether the second predefined amount of time elapsed may be performed. If computing system 130 and/or QC check system 550 determine that the second predefined amount of time has elapsed, then computing system 130 and/or QC check system 550 may perform the QC check to the iPSCs stored in the third plurality of sample containers.

In some embodiments, QC check system 550 may be configured to perform a QC check to the iPSCs stored within the selected sample container based on the second predefined amount of time having elapsed. The QC check may produce a QC score. The QC score may indicate whether the iPSCs stored in the selected sample container satisfy QC standards. The QC checks may include applying one or more stains to the sampled iPSCs, imaging the stained iPSCs, and determining whether those imaged iPSCs pass the QC, such as comparing the imaged iPSCs with a pre-determined threshold percentage of the total number of iPSCs that are positive for a marker of interest specific to a particular cell type.

In some embodiments, the QC score may be provided to computing system 130. QC check subsystem 142 may instruct QC check system 550 to discard the iPSCs stored in the selected sample container, as well as some or all of the lot of iPSCs, based on the QC score being less than a threshold QC score. QC check system 550 may then cause cell handling system 500 to transport the lot of iPSCs to cell discard system 560 for discarding. In some embodiments, QC check subsystem 142 instructs QC check system 550 to release the lot of iPSCs banked in cell storage based on the QC score being greater than or equal to the threshold QC score. After the lot of iPSCs has been released, they may be subjected to one or more additional cell differentiation steps to produce the desired differentiated cells. The lot of iPSCs, as mentioned above, may include the iPSCs stored in the second plurality of sample containers excluding the selected sample container, for which the QC check was performed.

In some embodiments, second differentiation stage subsystem 144 may instruct differentiation processing system 120 to perform one or more additional cell differentiation steps to the lot of iPSCs after being released to obtain a plurality of differentiated cells. The differentiated cells may be of a particular cell type desired for phenotyping. For example, the differentiated cells may be motor neurons, which can be used for clinical trials related to treatment of neurological conditions.

In some embodiments, second differentiation stage subsystem 144 may be configured to instruct cell handling system 500 to distribute the lot of iPSCs stored within the second plurality of sample containers across a third plurality of sample containers. The number of sample containers in the third plurality of sample containers may be less than or equal to the number of sample containers included in the first plurality of sample containers and/or the second plurality of sample containers. Each sample container of the third plurality of sample containers may include a substantially similar number of iPSCs. In some embodiments, second differentiation stage subsystem 144 may instruct cell feeding system 530 to subject the distributed subset of iPSCs in each of the third plurality of sample containers to one or more additional cell differentiation steps. These cell differentiation steps may include causing cell feeding system 530 to feed the iPSCs distributed within each sample container of the third plurality of sample containers. In some embodiments, cell feeding system 530 may perform the additional cell differentiation steps by providing a particular media to the iPSCs to induce differentiation towards a particular type of cell (e.g., a motor neuron). In some embodiments, the feeding may be performed at a particular frequency, such as every hour, every few hours, every day, every week, etc. In some embodiments, the feeding may include an additional compound, such as small molecules. For example, a given sample container of the third plurality of sample containers may already include iPSCs. The small molecules may be added to the sample container to elicit a phenotyping response.

In some embodiments, second differentiation stage subsystem 144 may instruct imaging system 540 to capture a fourth plurality of images depicting the distributed lot of iPSCs in each of the third plurality of sample containers. In some embodiments, second differentiation stage subsystem 144 may analyze the fourth plurality of images to determine whether the iPSCs are developing as expected. For example, these images may be used for routine inspection to ensure that seeding is uniform, and that the iPSCs stored in the sample containers have a target cell density/confluence. Furthermore, these images may be used to ensure that no contaminants are present in the sample containers.

In some embodiments, second differentiation stage subsystem 144 may be configured to determine whether a third predefined amount of time has elapsed from the feeding of the distributed subsets of iPSCs in each of the third plurality of sample containers. If the third predefined amount of time has not elapsed, second differentiation stage subsystem 144 may instruct cell feeding system 530 to perform another feeding to the iPSCs. However, if the third predefined amount of time has elapsed, second differentiation stage subsystem 144 may instruct cell handling system to transport the differentiated cells to phenotyping system 570. In some embodiments, the third predefined amount of time may be the same or similar to the second predefined amount of time (e.g., 10 days). The amount of time may be selected such that progenitors can sufficiently expand.

In some embodiments, second differentiation stage subsystem 144 may be configured to assess a quality of a differentiation as it proceeds. Second differentiation stage subsystem 144 may implement a machine learning model trained to assess the quality of the differentiation. In some embodiments, the machine learning models may assess the quality of the differentiation using the fourth plurality of images depicting the iPSCs stored in the third plurality of sample containers. The machine learning model may be stored in model database 152. This machine learning model may be cell type specific. As an example, to train a machine learning model to assess the differentiation for motor neurons, a number of motor neuron differentiations may be performed to obtain training data describing failed and successful differentiations. The training data may be stored in training data database 154. The training data may include the failed/successful differentiation information matched with QPC imaging or bright-field imaging. The training data may be used to train a machine learning model to predict whether a differentiation step will be successful as the differentiation is occurring. In some embodiments, the machine learning models used to determine the quality of the differentiation may be the same or similar for both first differentiation stage subsystem 140 and second differentiation stage subsystem 144.

Phenotyping system 570 may be configured to perform one or more phenotypic assessments using some or all of the differentiated cells. In some embodiments, phenotyping subsystem 146 may instruct cell handling system 500 to transport the iPSCs stored in the third plurality of sample containers to phenotyping system 570. In some embodiments, phenotyping system 570 may fix the differentiated cells using PFA. Phenotyping system 570 may then stain the fixed cells using one or more indirect immunofluorescence techniques. For example, the immunofluorescence techniques may be performed with antibodies for markers HB9/B3tubulin/ISL1, which are markers specific to motor neurons. In some embodiments, phenotyping subsystem 146 may prepare the differentiated cells via RNAseq analysis. The RNAseq analysis may be a bulk RNAseq or an scRNAseq.

In some embodiments, the differentiated cells may be transported to a different vessel. For example, imaging assays may keep the differentiated cells in the same multi-sample plate as during the second round of differentiation. However, an assay that looks at protein levels or transcript levels (e.g., RNAseq) may include the differentiated cells being transported to different vessels.

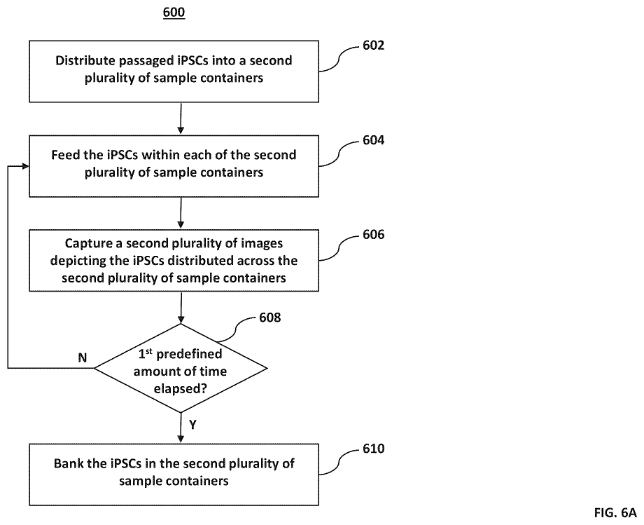

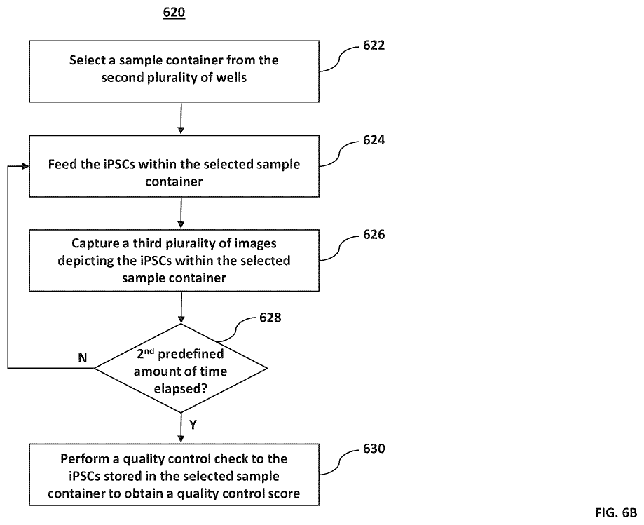

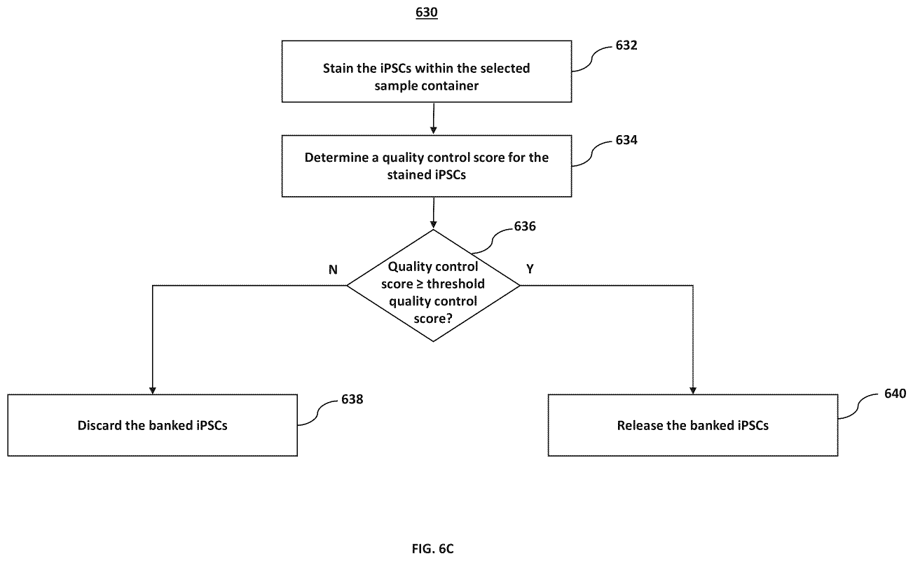

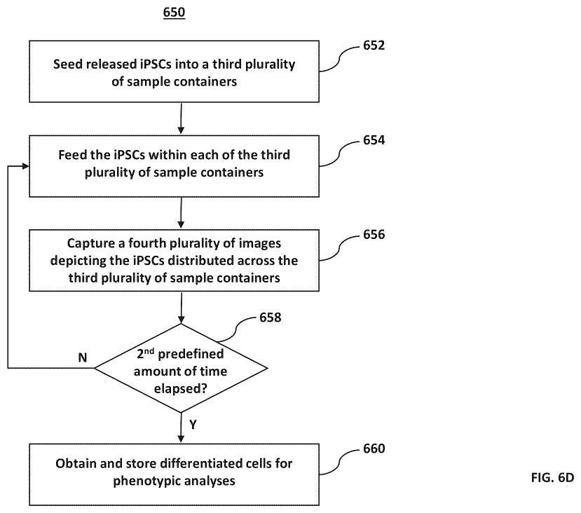

FIGS. 6A-6D illustrate exemplary methods for subjecting iPSCs to one or more cell differentiation steps, in accordance with some embodiments. For example, FIG. 6A illustrates an example method 600 for performing a first cell differentiation. FIG. 6B may illustrate an example method 620 for preparing cells for a quality control check. FIG. 6C may illustrate an example of operation 630 of method 620 for performing the quality control check. FIG. 6D may illustrate an example method 650 for performing a second cell differentiation. Methods 600, 620, and 650, and the operations encompassed thereby, may be performed, for example, using one or more electronic devices implementing a software platform, such as AMD platform 100. In some examples, methods 600, 620, and 650 may be performed using a client-server system, and the steps of methods 600, 620, and 650 may be divided up in any manner between the server (e.g., AMD platform 100) and one or more client devices (e.g., client device 102). Thus, while portions of methods 600, 620, and 650 are described herein as being performed by particular devices of a client-server system, it will be appreciated that methods 600, 620, and 650 are not so limited. In other examples, methods 600, 620, and 650 may be performed using only a client device or only multiple client devices. In methods 600, 620, and 650, some blocks are, optionally, combined, the order of some blocks is, optionally, changed, and some blocks are, optionally, omitted. In some examples, additional steps may be performed in combination with methods 600, 620, and 650. Accordingly, the operations as illustrated (and described in greater detail below) are exemplary by nature and, as such, should not be viewed as limiting.

In some embodiments, method 600 may begin at operation 602. In operation 602, iPSCs that have been passaged may be distributed across a second plurality of sample containers (e.g., a plurality of sample wells on a multi-well plate). The iPSCs may include those stored in the at least one selected sample container of the first plurality of sample containers. In some embodiments, the second plurality of sample containers may include the same or a different number of sample containers as the first plurality of sample containers. For example, the first plurality of sample containers may include ninety-six sample containers (e.g., a 96-well plate) and the second plurality of sample containers may include twelve sample containers (e.g., a 12-well plate). The number of iPSCs stored in each sample container of the second plurality of sample containers may be the same or similar to that of the first plurality of sample containers. In some embodiments, these iPSCs may be retrieved from cell storage where they may be kept frozen.

In operation 604, the iPSCs stored within each of the second plurality of sample containers may be fed. In some embodiments, the feeding may be part of a first differentiation stage. During the first differentiation stage, a media that induces differentiation of the cells may be provided to the iPSCs stored in the second plurality of sample containers. For example, a cell type specific media that induces differentiation toward a motor neuron cell type may be used. In some embodiments, the feeding may be performed at a particular cadence. For example, the feeding may be performed every 24 hours. In between feedings the iPSCs may be incubated, for example, at 37° C.

In operation 606, a second plurality of images depicting the iPSCs distributed across the second plurality of sample containers may be captured. In some embodiments, imaging system 540 may be configured to capture the second plurality of images depicting some or all of the second plurality of sample containers. In some embodiments, the second plurality of images may be provided to computing system 130 for analysis. In some embodiments, computing system 130 may be configured to assess a quality of a differentiation as it proceeds. Machine learning models stored in model database 152 may be employed to assess the quality of the differentiation and predict whether the cell differentiation process will yield differentiated cells.

In operation 608, a determination may be made as to whether a first predefined amount of time has elapsed from the feeding of iPSCs stored in the second plurality of sample containers. If, in operation 608, it is determined that the first predefined amount of time has not elapsed, method 600 may return to operation 602 where the iPSCs may be fed again. However, if, at operation 608, it is determined that the first predefined amount of time has elapsed, then method 600 may proceed to operation 610. In operation 610, the iPSCs stored in the second plurality of sample containers may be banked. The banked iPSCs may be referred to as a “lot” of iPSCs. In some embodiments, the first predefined amount of time may be three days, however other amounts of time may be used (e.g., 1 or more days, 5 or more days, 10 or more days, etc.).

In some embodiments, method 620 may begin at operation 622. In operation 622, a sample container from the second plurality of wells may be selected. The selection may be random. The iPSCs stored in the selected sample container may be prepared for a QC check.

In operation 624, the iPSCs stored within the selected sample container may be fed. The feeding may be the same or similar to the feeding performed during the first differentiation. In some embodiments, the iPSCs stored within the selected sample container may be distributed across a plurality of sample containers (e.g., a plurality of wells in a multi-well plate). The number of sample containers for the QC check may be less than the number of sample containers used during cell maintenance and/or the first differentiation stage.

In operation 626, a third plurality of images depicting the iPSCs within the selected sample container may be captured. In some embodiments, the third plurality of images may depict the iPSCs stored in the plurality of sample containers at operation 624. The third plurality of images may be captured by imaging system 540, and the third plurality of images may be provided to computing system 130 for analysis. In some embodiments, the plurality of images may be provided to computing system 130 for analysis. In some embodiments, computing system 130 may be configured to assess a quality of a differentiation as it proceeds. Machine learning models stored in model database 152 may be employed to assess the quality of the differentiation.

In operation 628, a determination may be made as to whether a second predefined amount of time has elapsed from the feeding of iPSCs stored in the plurality of sample containers. If, in operation 628, it is determined that the second predefined amount of time has not elapsed, method 620 may return to operation 624 where the iPSCs may be fed again. However, if, at operation 628, it is determined that the second predefined amount of time has elapsed, then method 620 may proceed to operation 630. In operation 630, a quality control check may be performed to the iPSCs stored in the selected sample container to obtain a quality control score. Operation 630 is further detailed below. In some embodiments, the first predefined amount of time may be three days, however other amounts of time may be used (e.g., 1 or more days, 5 or more days, 10 or more days, etc.).

In some embodiments, operation 630 may include a set of sub-operations. Operation 630 may comprise sub-operations for performing a QC check. The QC check may serve as a proxy for the lot of iPSCs stored in the banked sample containers. As illustrated in FIG. 6C, operation 630 may begin at a sub-operation 632. In sub-operation 632, the iPSCs within the selected sample container of the second plurality of sample containers (i.e., operation 622) may be stained. The stain may include a marker, such as OCT4, NANOG, or others.

In sub-operation 634, a QC score for the stained iPSCs may be determined. In some embodiments, one or more machine learning models may be used to determine the QC score. In sub-operation 636, a determination may be made as to whether the QC score is greater than or equal to a threshold QC score. In some embodiments, the threshold QC score may be 75% or greater, 85% or greater, 95% or greater, or other values.

If, in sub-operation 636, it was determined that the QC score is less than the threshold QC score, then operation 630 may proceed to sub-operation 638. In sub-operation 638, the lot of iPSCs banked in cell storage may be discarded. This may be because the tested iPSCs do not pass the threshold QC score, and therefore the remaining iPSCs of the lot are unlikely to develop into usable differentiated cells. However, if, in sub-operation 636, it was determined that the QC score is greater than or equal to the threshold QC score, then operation 630 may proceed to sub-operation 640. In sub-operation 640, the lot of iPSCs banked in cell storage may be released. The released lot of iPSCs may then be subjected to one or more additional cell differentiation steps.

In some embodiments, method 650 may begin at operation 652. In operation 652, the lot of iPSCs banked in cell storage may be seeded across a third plurality of sample containers. The lot of iPSCs that are distributed across the third plurality of sample containers may exclude the iPSCs selected for the QC check.

In operation 654, the iPSCs stored within the third plurality of sample containers may be fed. The feeding may be the same or similar to the feeding performed during the first differentiation. The third plurality of sample containers may include the same number of sample containers used during the first differentiation stage.

In operation 656, a fourth plurality of images depicting the iPSCs stored within the third plurality of sample containers may be captured. In some embodiments, the fourth plurality of images may depict the iPSCs distributed amongst the third plurality of sample containers. The third plurality of images may be captured by imaging system 540, and the fourth plurality of images may be provided to computing system 130 for analysis. In some embodiments, computing system 130 may be configured to assess a quality of a differentiation as it proceeds. Machine learning models stored in model database 152 may be employed to assess the quality of the differentiation.

In operation 658, a determination may be made as to whether the second predefined amount of time has elapsed from the feeding of iPSCs stored in the third plurality of sample containers. If, in operation 658, it is determined that the second predefined amount of time has not elapsed, method 650 may return to operation 654 where the iPSCs may be fed again. However, if, at operation 658, it is determined that the second predefined amount of time has elapsed, then method 650 may proceed to operation 660. In operation 660, the differentiated cells may be obtained. These differentiated cells may be stored in cell storage for later use during one or more phenotypic analyses.

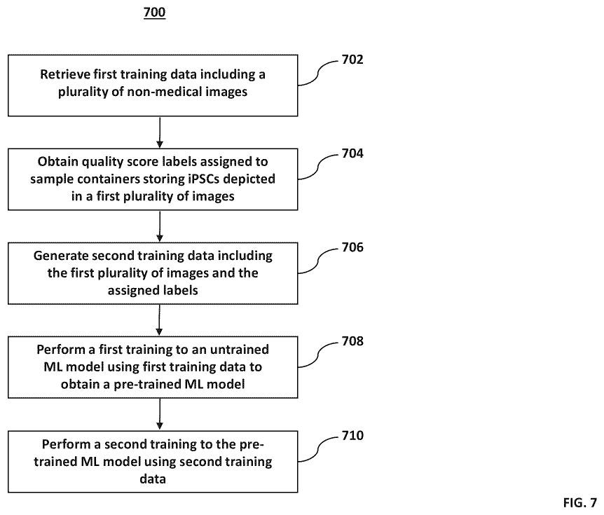

FIG. 7 illustrates an example method 700 for training one or more machine learning models to determine a quality score of sample containers storing iPSCs, in accordance with various embodiments. Method 700 is performed, for example, using one or more electronic devices implementing a software platform, such as AMD platform 100. In some examples, method 700 is performed using a client-server system, and the steps of method 700 are divided up in any manner between the server (e.g., AMD platform 100) and one or more client devices (e.g., client device 102). Thus, while portions of method 700 are described herein as being performed by particular devices of a client-server system, it will be appreciated that method 700 is not so limited. In other examples, method 700 is performed using only a client device or only multiple client devices. In method 700, some blocks are, optionally, combined, the order of some blocks is, optionally, changed, and some blocks are, optionally, omitted. In some examples, additional steps may be performed in combination with method 700. Accordingly, the operations as illustrated (and described in greater detail below) are exemplary by nature and, as such, should not be viewed as limiting.

In some embodiments, method 700 may begin at operation 702. In operation 702, first training data may be retrieved, where the first training data may include a plurality of non-medical images. For example, the plurality of non-medical images may include images from the ImageNet database. The first training data may be stored in training data database 154. In some embodiments, operation 702 may be performed by a subsystem that is the same as or similar to model training subsystem 138.

In operation 704, quality score labels assigned to each sample container of a first plurality of sample containers may be obtained. Each sample container may store a subset of iPSCs to be used to produce differentiated cells. In some embodiments, the first plurality of sample containers may be imaged using an imaging system (e.g., imaging system 340) to obtain a first plurality of images. In some embodiments, the iPSCs stored in the first plurality of sample containers may be stained to highlight a location of the cells within the sample containers. For example, a DAPI stain may be applied to the iPSCs. In some embodiments, each of the first plurality of images may be tiled. For example, the first plurality of images may be whole slide images, which may be tiled to produce a set of tiles for each of the first plurality of images. Each tile may have a smaller size as compared to the whole slide image from which it was derived. The tiles may be overlapping or non-overlapping.

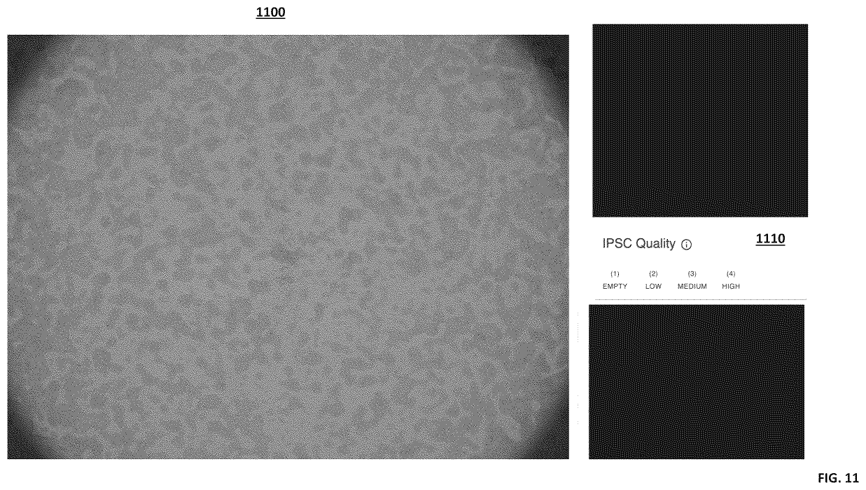

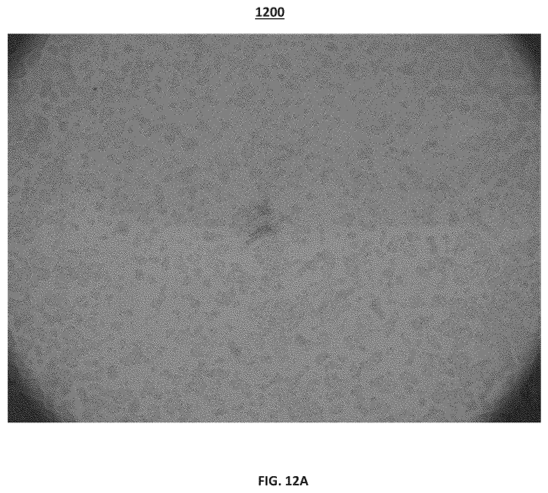

In some embodiments, the first plurality of images may be presented to an individual, such as a trained pathologist, via a web interface. For example, the first plurality of images may be presented to an individual using a web application rendered on their client device 102. Using the web application (or another interface), the individual may provide a quality score label for each sample container. As an example, with reference to FIG. 11, image 1100 may depict an image of a sample container storing iPSCs that may be presented to the trained pathologies via the web interface. The web interface may include a feedback section 1110 where the individual can input their quality score classification for image 1100. For example, the individual may view image 1100 of a sample container and assign a quality score to that sample container and/or classify the images into one of the quality score classifications (e.g., high-quality classification, medium-quality classification, low-quality classification, empty). In some embodiments, multiple sets of quality score labels may be obtained. Multiple individuals, such as multiple trained pathologists, may each be provided with the first plurality of images, and each individual may independently assign a quality score to the sample containers depicted within the first plurality of images. For example, a first user and a second user may each be presented with an image of a sample container storing a subset of iPSCs. The first user may assess the sample container as having a first quality score and the second user may assess the sample container as having a second, different, quality score. In some embodiments, operation 704 may be performed by a subsystem that is the same as or similar to model training subsystem 138.

In operation 706, second training data including the first plurality of images and the quality score labels assigned to the images may be generated. In some embodiments, the second training data may associate each image of the first plurality of images with a quality score label. For example, a first image may be assigned the quality score “high-quality” by the first user and the quality score “medium-quality” by the second user. The second training data may therefore include two data points, the first pairing the first image (or an embedding generated from that image) with a first label representing the “high-quality” score, and the second pairing the first image with a second label representing the “medium-quality” score. In some embodiments, operation 706 may be performed by a subsystem that is the same as or similar to model training subsystem 138.

In operation 708, a first training may be performed to an untrained ML model using the first training data to obtain a pre-trained ML model. For example, the first training data may be used to tune one or more hyperparameters of the ML model. In one embodiment, the first training data may be used to obtain a pre-trained model (e.g., obtain an initial set of trained values so that the ML model produces accurate embeddings). In some embodiments, the pre-trained model may have a ResNet-18 architecture, and an Adam optimizer may be used during the training. In some embodiments, operation 708 may be optional, and the pre-trained ML model may be stored in model database 152 and retrieved for fine-tuning. In some embodiments, operation 708 may be performed by a subsystem that is the same as or similar to model training subsystem 138.

In operation 710, a second training may be performed to the pre-trained ML model using the second training data. The second training data may be used to train the pre-trained ML model to predict/estimate a quality score for sample containers storing iPSCs depicted by an image. In some embodiments, the second training may also use the Adam optimizer. The second training may be repeated a predefined number of times or until an accuracy of the model satisfies an accuracy threshold condition (e.g., the model predicts a quality score with an accuracy greater than or equal to a threshold accuracy). The trained ML model may be stored in model database 152. In some embodiments, operation 710 may be performed by a subsystem that is the same as or similar to model training subsystem 138.

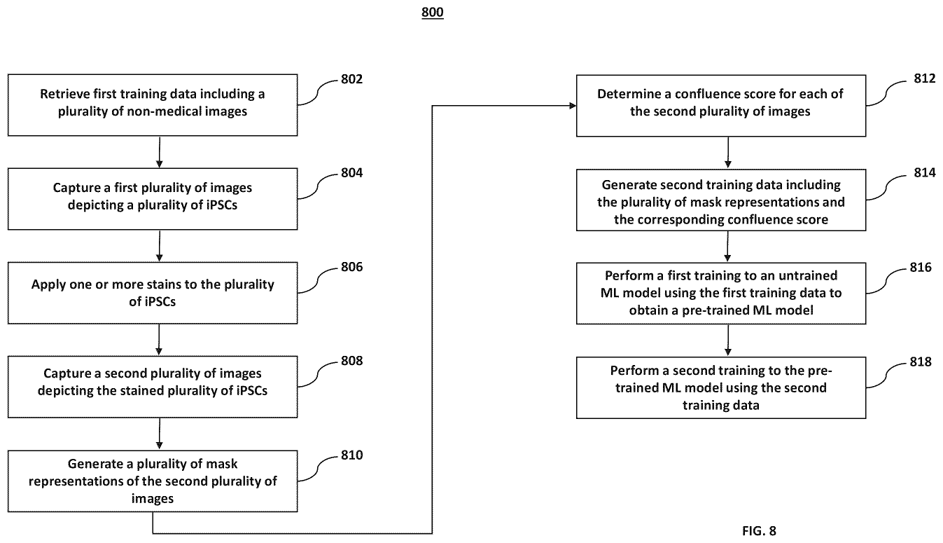

FIG. 8 illustrates an example method 800 for training one or more machine learning models to determine a confluence score of sample containers storing iPSCs, in accordance with some embodiments. Method 800 is performed, for example, using one or more electronic devices implementing a software platform, such as AMD platform 100. In some examples, method 800 is performed using a client-server system, and the steps of method 800 are divided up in any manner between the server (e.g., AMD platform 100) and one or more client devices (e.g., client device 102). Thus, while portions of method 800 are described herein as being performed by particular devices of a client-server system, it will be appreciated that method 800 is not so limited. In other examples, method 800 is performed using only a client device or only multiple client devices. In method 800, some blocks are, optionally, combined, the order of some blocks is, optionally, changed, and some blocks are, optionally, omitted. In some examples, additional steps may be performed in combination with method 800. Accordingly, the operations as illustrated (and described in greater detail below) are exemplary by nature and, as such, should not be viewed as limiting.

In some embodiments, method 800 may begin at operation 802. In operation 802, first training data may be retrieved, where the first training data may include a plurality of non-medical images. For example, the plurality of non-medical images may include images from the ImageNet database. The first training data may be stored in training data database 154. In some embodiments, operation 802 may be performed by a subsystem that is the same as or similar to model training subsystem 138.

In operation 804, a first plurality of images depicting iPSCs distributed across sample containers may be captured. In some embodiments, the first plurality of images may be captured using imaging system 340. Each sample container may store a subset of iPSCs to be developed into differentiated cells. In some embodiments, the first plurality of images may be images captured without one or more stains applied to the iPSCs. In some embodiments, each of the first plurality of images may be tiled. For example, the first plurality of images may be whole slide images, which may be tiled to produce a set of tiles for each of the first plurality of images. Each tile may have a smaller size as compared to the whole slide image from which it was derived. The tiles may be overlapping or non-overlapping. In some embodiments, operation 804 may be performed by a subsystem that is the same as or similar to model training subsystem 138.

In operation 806, a stain may be applied to some or all of the iPSCs stored in the sample containers. In some embodiments, the iPSCs stored in the sample containers may be stained to highlight a location of the cells within the sample containers. For example, a DAPI stain may be applied to the iPSCs. In operation 808, a second plurality of images depicting the stained iPSCs may be captured. The second plurality of images may also depict the iPSCs stored in the sample containers, however the iPSCs may not have a stain applied to them (e.g., a DAPI stain). In some embodiments, each of the second plurality of images may be tiled. For example, the second plurality of images may be whole slide images, which may be tiled to produce a set of tiles for each of the second plurality of images. Each tile may have a smaller size as compared to the whole slide image from which it was derived. The tiles may be overlapping or non-overlapping. In some embodiments, operations 806 and 808 may be performed by a subsystem that is the same as or similar to model training subsystem 138.

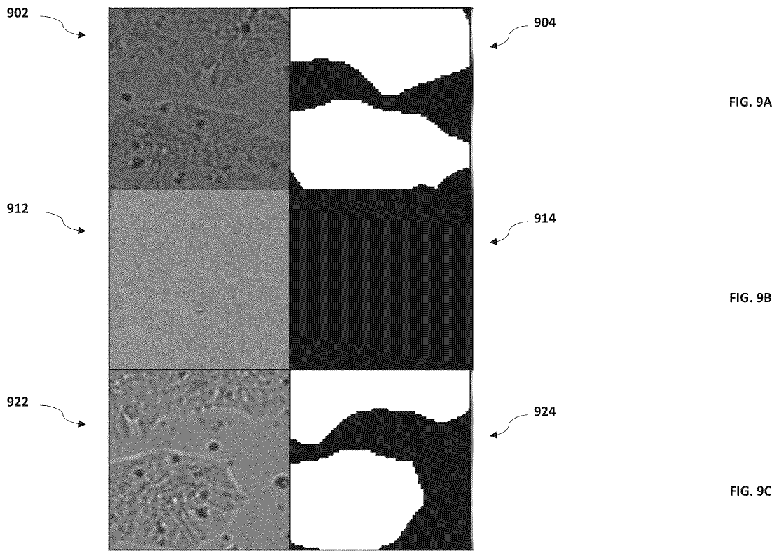

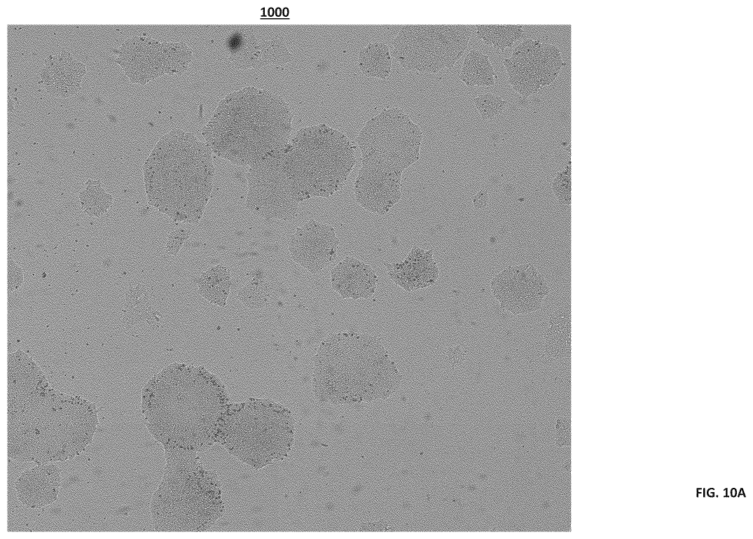

In operation 810, a plurality of mask representations of the second plurality of images may be generated. The mask representations may be generated manually and/or with the aid of computer vision models. The mask representations may indicate which pixels of an image of a sample container depict one or more alive iPSCs and which pixels of the image do not depict one or more alive iPSCs (e.g., depict other contents, such as one or more dead iPSCs). An example bright-field image and a corresponding mask representation generated for that image is illustrated in FIGS. 9A-9C. As seen in FIGS. 9A-9C, each of bright-field images 902, 912, and 922 may have a corresponding mask representation 904, 914, and 924. The pixels of mask representations 904, 914, and 924 in white may represent portions of bright-field images 902, 912, and 922, respectively, occupied by one or more alive iPSCs. The pixels of mask representations 904, 914, and 924 in black may represent portions of bright-field images 902, 912, and 922, respectively, not occupied by one or more alive iPSCs. Using the first plurality of images and the second plurality of images, mask representations may be generated for training a machine learning model to predict a confluence score for a sample container storing iPSCs. In some embodiments, operation 810 may be performed by a subsystem that is the same as or similar to model training subsystem 138.

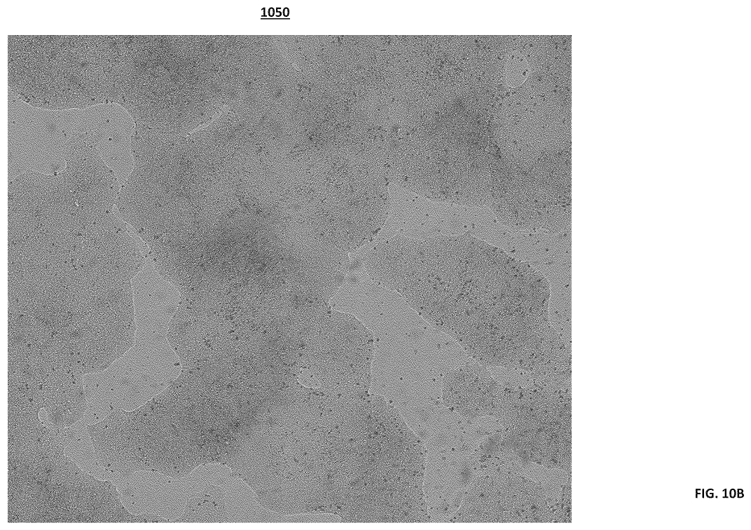

In operation 812, a confluence score for each of the second plurality of images may be determined. The confluence score computed for each of the second plurality of images may indicate how “occupied” a sample container is with alive iPSCs, compared to other contents (e.g., one or more dead iPSCs, debris, condensation, cell media not containing iPSCs, etc.). The alive iPSCs may develop in clusters. Therefore, confluence is a measure of the percentage of surface area covered by alive iPSCs. Sample containers having low alive iPSC occupancy may have a low confluence score, whereas sample containers having high alive iPSC occupancy may have a high confluence score. As an example, with reference to FIGS. 10A-10B, images 1000 and 1050 may depict examples images of a sample container storing iPSCs having different confluence scores. For example, image 1000 may depict a sample container having a confluence score of 30%, whereas image 1050 may depict a sample container having a confluence score of 80%. In some embodiments, operation 812 may be performed by a subsystem that is the same as or similar to model training subsystem 138.

In operation 814, second training data including the plurality of mask representations and the corresponding confluence score may be generated. The second training data may also include the bright-field images and the DAPI stained images used to produce the mask representations. For example, the second training data may include tuples including a bright-field image, its corresponding DAPI-stained image, a mask representation generated for the bright-field image, and the confluence score computed for those image pairs. In some embodiments, the second training data may also store embeddings generated for the bright-field image and/or the DAPI-stained image. In some embodiments, operation 814 may be performed by a subsystem that is the same as or similar to model training subsystem 138.

In operation 816, a first training may be performed to an untrained ML model using the first training data to obtain a pre-trained ML model. For example, the first training data may be used to tune one or more hyperparameters of the ML model. In some embodiments, the ML model is not pretrained. In some embodiments, the ML model or pre-trained ML model may have a ResNet-18 architecture, and an Adam optimizer may be used during the training. In some embodiments, the ML model or pre-trained ML model may have a UNet architecture. In some embodiments, operation 816 may be optional, and the ML model or pre-trained ML model may be stored in model database 152 and retrieved for fine-tuning. In some embodiments, operation 816 may be performed by a subsystem that is the same as or similar to ML model training subsystem 138.

In operation 818, a second training may be performed to the pre-trained ML model using the second training data. The second training data may be used to train the pre-trained ML model to predict/estimate a confluence score for sample containers storing iPSCs depicted by an image. In some embodiments, the second training may also use the Adam optimizer. The second training may be repeated a predefined number of times or until an accuracy of the model satisfies an accuracy threshold condition (e.g., the model predicts a confluence score with an accuracy greater than or equal to a threshold accuracy). The trained ML model may be stored in model database 152. In some embodiments, operation 818 may be performed by a subsystem that is the same as or similar to model training subsystem 138.

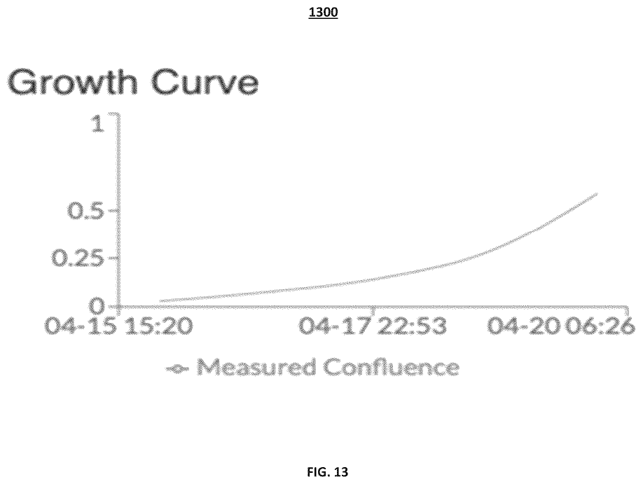

FIG. 13 is an illustrative plot 1300 of a growth curve depicting a change in confluence of a sample container over time, in accordance with various embodiments. The growth curve represents the rate of change of the confluence score of a sample container over time. In some embodiments, the confluence score for a sample container may be determined at a predefined frequency (e.g., daily), and each point along the growth curve represents the confluence score for that sample container at each interval. The intrinsic growth rate of each cell line may be a reflection of the doubling time of the iPSCs stored within a sample container. In some embodiments, the growth curve may be used to estimate a confluence score, and the estimated confluence score may be used to predict downstream characteristics of the iPSCs. In some embodiments, there may be a linear relationship between the confluence score of a sample container and the quality score of that same sample container.

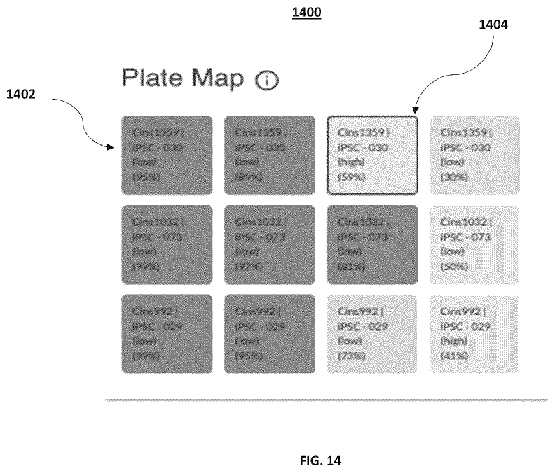

FIG. 14 illustrates an example sample plate map 1400 including information about a plurality of sample containers, in accordance with various embodiments. Sample plate map 1400 may include twelve legends, each corresponding to a sample container of a multi-sample plate. The sample containers of the multi-sample plate may be fed and imaged, and the images depicting the sample containers may be analyzed using one or more machine learning models to determine a quality score and/or a confluence score of each. In some embodiments, the legends may include quality score classifications and/or confluence score classifications for each sample container. For example, legend 1402 may indicate that a first sample container has a low-quality score, and thus has been classified into the low-quality classification. Legend 1402 may also indicate that the first sample container has a confluence score of 95%, however in some cases the confluence score classification may be included within legend 1402. As another example, legend 1404 may indicate that a second sample container has a high-quality score, and thus has been classified into the high-quality classification. Legend 1404 may also indicate that the second sample container has a confluence score of 59%, however in some cases the confluence score classification may be included within legend 1404.

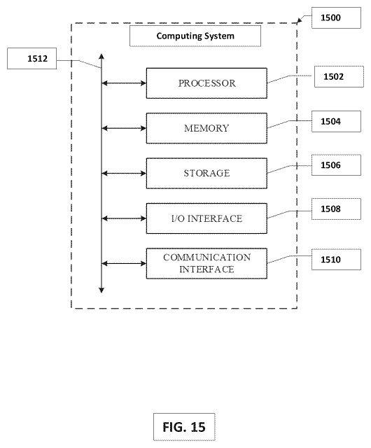

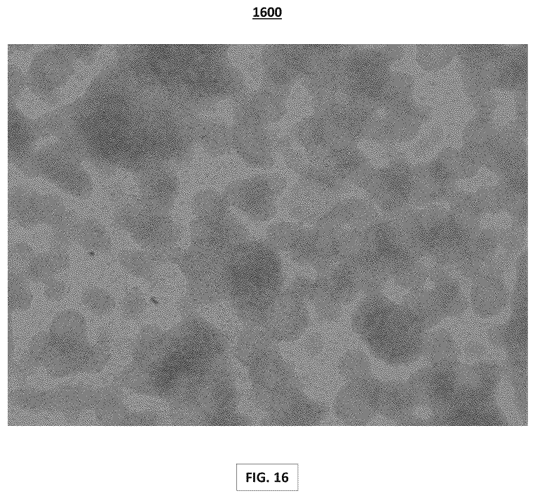

FIG. 15 illustrates an example computing system 1500. In particular embodiments, one or more computing systems 1500 perform one or more steps of one or more methods described or illustrated herein. In particular embodiments, one or more computing systems 1500 provide functionality described or illustrated herein. In particular embodiments, software running on one or more computing systems 1500 performs one or more steps of one or more methods described or illustrated herein or provides functionality described or illustrated herein. Particular embodiments include one or more portions of one or more computing systems 1500. Herein, reference to a computer system may encompass a computing device, and vice versa, where appropriate. Moreover, reference to a computer system may encompass one or more computer systems, where appropriate.

This disclosure contemplates any suitable number of computing systems 1500. This disclosure contemplates computing system 1500 taking any suitable physical form. As example and not by way of limitation, computing system 1500 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computing system 1500 may include one or more computing systems 1500; be unitary or distributed; span multiple locations; span multiple machines; span multiple data centers; or reside in a cloud, which may include one or more cloud components in one or more networks. Where appropriate, one or more computing systems 1500 may perform without substantial spatial or temporal limitation one or more steps of one or more methods described or illustrated herein. As an example, and not by way of limitation, one or more computing systems 1500 may perform in real time or in batch mode one or more steps of one or more methods described or illustrated herein. One or more computing systems 1500 may perform at different times or at different locations one or more steps of one or more methods described or illustrated herein, where appropriate.

In particular embodiments, computing system 1500 includes a processor 1502, memory 1504, storage 1506, an input/output (I/O) interface 1508, a communication interface 1510, and a bus 1512. Although this disclosure describes and illustrates a particular computer system having a particular number of particular components in a particular arrangement, this disclosure contemplates any suitable computer system having any suitable number of any suitable components in any suitable arrangement.

In particular embodiments, processor 1502 includes hardware for executing instructions, such as those making up a computer program. As an example, and not by way of limitation, to execute instructions, processor 1502 may retrieve (or fetch) the instructions from an internal register, an internal cache, memory 1504, or storage 1506; decode and execute them; and then write one or more results to an internal register, an internal cache, memory 1504, or storage 1506. In particular embodiments, processor 1502 may include one or more internal caches for data, instructions, or addresses. This disclosure contemplates processor 1502 including any suitable number of any suitable internal caches, where appropriate. As an example, and not by way of limitation, processor 1502 may include one or more instruction caches, one or more data caches, and one or more translation lookaside buffers (TLBs). Instructions in the instruction caches may be copies of instructions in memory 1504 or storage 1506, and the instruction caches may speed up retrieval of those instructions by processor 1502. Data in the data caches may be copies of data in memory 1504 or storage 1506 for instructions executing at processor 1502 to operate on; the results of previous instructions executed at processor 1502 for access by subsequent instructions executing at processor 1502 or for writing to memory 1504 or storage 1506; or other suitable data. The data caches may speed up read or write operations by processor 1502. The TLBs may speed up virtual-address translation for processor 1502. In particular embodiments, processor 1502 may include one or more internal registers for data, instructions, or addresses. This disclosure contemplates processor 1502 including any suitable number of any suitable internal registers, where appropriate. Where appropriate, processor 1502 may include one or more arithmetic logic units (ALUs); be a multi-core processor; or include one or more processors 1502. Although this disclosure describes and illustrates a particular processor, this disclosure contemplates any suitable processor.

In particular embodiments, memory 1504 includes main memory for storing instructions for processor 1502 to execute or data for processor 1502 to operate on. As an example, and not by way of limitation, computing system 1500 may load instructions from storage 1506 or another source (such as, for example, another computing system 1500) to memory 1504. Processor 1502 may then load the instructions from memory 1504 to an internal register or internal cache. To execute the instructions, processor 1502 may retrieve the instructions from the internal register or internal cache and decode them. During or after execution of the instructions, processor 1502 may write one or more results (which may be intermediate or final results) to the internal register or internal cache. Processor 1502 may then write one or more of those results to memory 1504. In particular embodiments, processor 1502 executes only instructions in one or more internal registers or internal caches or in memory 1504 (as opposed to storage 1506 or elsewhere) and operates only on data in one or more internal registers or internal caches or in memory 1504 (as opposed to storage 1506 or elsewhere). One or more memory buses (which may each include an address bus and a data bus) may couple processor 1502 to memory 1504. Bus 1512 may include one or more memory buses, as described below. In particular embodiments, one or more memory management units (MMUs) reside between processor 1502 and memory 1504 and facilitate accesses to memory 1504 requested by processor 1502. In particular embodiments, memory 1504 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1504 may include one or more memories 1504, where appropriate. Although this disclosure describes and illustrates particular memory, this disclosure contemplates any suitable memory.

In particular embodiments, storage 1506 includes mass storage for data or instructions. As an example, and not by way of limitation, storage 1506 may include a hard disk drive (HDD), a floppy disk drive, flash memory, an optical disc, a magneto-optical disc, magnetic tape, or a Universal Serial Bus (USB) drive or a combination of two or more of these. Storage 1506 may include removable or non-removable (or fixed) media, where appropriate. Storage 1506 may be internal or external to computing system 1500, where appropriate. In particular embodiments, storage 1506 is non-volatile, solid-state memory. In particular embodiments, storage 1506 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 1506 taking any suitable physical form. Storage 1506 may include one or more storage control units facilitating communication between processor 1502 and storage 1506, where appropriate. Where appropriate, storage 1506 may include one or more storages 1506. Although this disclosure describes and illustrates particular storage, this disclosure contemplates any suitable storage.

In particular embodiments, I/O interface 1508 includes hardware, software, or both, providing one or more interfaces for communication between computing system 1500 and one or more I/O devices. Computing system 1500 may include one or more of these I/O devices, where appropriate. One or more of these I/O devices may enable communication between a person and computing system 1500. As an example, and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1508 for them. Where appropriate, I/O interface 1508 may include one or more device or software drivers enabling processor 1502 to drive one or more of these I/O devices. I/O interface 1508 may include one or more I/O interfaces 1508, where appropriate. Although this disclosure describes and illustrates a particular I/O interface, this disclosure contemplates any suitable I/O interface.

In particular embodiments, communication interface 1510 includes hardware, software, or both providing one or more interfaces for communication (such as, for example, packet-based communication) between computing system 1500 and one or more other computing systems 1500 or one or more networks. As an example, and not by way of limitation, communication interface 1510 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1510 for it. As an example, and not by way of limitation, computing system 1500 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computing system 1500 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computing system 1500 may include any suitable communication interface 1510 for any of these networks, where appropriate. Communication interface 1510 may include one or more communication interfaces 1510, where appropriate. Although this disclosure describes and illustrates a particular communication interface, this disclosure contemplates any suitable communication interface.

In particular embodiments, bus 1512 includes hardware, software, or both coupling components of computing system 1500 to each other. As an example and not by way of limitation, bus 1512 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1512 may include one or more buses 1512, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (Ics) (such, as for example, field-programmable gate arrays (FPGAs) or application-specific Ics (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

What is claimed is:

1. A system for autonomous selection and maintenance of induced pluripotent stem cells (iPSCs), the system comprising: one or more processors, a memory, and one or more programs, wherein the one or more programs are stored in the memory and configured to be executed by the one or more processors, the one or more programs including instructions for:
capturing a first plurality of images depicting a plurality of iPSCs stored within a first plurality of sample containers using an imaging system;
determining, using one or more machine learning models, a confluence score for each sample container of the first plurality of sample containers based on the first plurality of images, the confluence score representing a confluence of the iPSCs of the plurality of iPSCs distributed within each sample container;
autonomously selecting at least one sample container from the first plurality of sample containers based on the confluence score of each of the first plurality of sample containers, wherein the at least one selected sample container comprises a subset of iPSCs of the plurality of iPSCs; and
autonomously performing one or more maintenance operations on the subset of iPSCs stored within the at least one selected sample container.

2. The system of claim 1, wherein the one or more programs further include instructions for:
determining, using the one or more machine learning models, a quality score for each sample container of the first plurality of sample containers based on the first plurality of images, the quality score representing a quality of the iPSCs of the plurality of iPSCs distributed within each sample container,
wherein autonomously selecting the at least one sample container comprises: autonomously selecting the at least one sample container from the first plurality of sample containers based on the confluence score and the quality score of each of the first plurality of sample containers.

3. The system of claim 2, wherein the quality score comprises a binary value, a numeric value, a classification, or any combination thereof.

4. The system of claim 3, wherein the quality score comprises one of: a low-quality score, a medium-quality score, or a high-quality score.

5. The system of claim 2, wherein the one or more programs further include instructions for:
determining a growth metric for each sample container of the first plurality of sample containers based on the first plurality of images, the growth metric representing a growth status of the iPSCs of the plurality of iPSCs distributed within each sample container,
wherein autonomously selecting the at least one sample container comprises: autonomously selecting the at least one sample container from the first plurality of sample containers based on the confluence score, the quality score, and the growth metric of each of the first plurality of sample containers.

6. The system of claim 5, wherein the growth metric is indicative of whether the iPSCs of the plurality of iPSCs distributed within each sample container is in a growth phase.

7. The system of claim 5, wherein the growth metric is indicative of a growth rate of the iPSCs of the plurality of iPSCs distributed within each sample container.

8. The system of claim 7, wherein the growth metric is indicative of whether the growth rate of the iPSCs of the plurality of iPSCs distributed within each sample container is positive.

9. The system of claim 5, wherein the growth metric is determined based on a first confluence score and a second confluence score of the plurality of iPSCs distributed within each sample container, wherein the first confluence score is associated with a first time point, and wherein the second confluence score is associated with a second time point later than the first time point.

10. The system of claim 9, wherein the growth metric indicates positive growth if the second confluence score is higher than the first confluence score.

11. The system of claim 5, wherein autonomously selecting the at least one sample container from the first plurality of sample containers comprises:
obtaining a ranking of a plurality of predefined confluence score ranges, wherein the plurality of predefined confluence score ranges comprises a first predefined confluence score range ranked higher than a second predefined confluence score range; and
prioritizing selection of a sample having a confluence score in the first predefined confluence score range over a sample having a confluence score in the second predefined confluence score range.

12. The system of claim 11, wherein autonomously selecting the at least one sample container from the first plurality of sample containers comprises: prioritizing selection of a sample having a higher quality score over a sample having a lower quality score.

13. The system of claim 11, wherein autonomously selecting the at least one sample container from the first plurality of sample containers comprises: prioritizing selection of a sample having a higher or positive growth metric over a sample having a lower or negative growth metric.

14. The system of claim 11, wherein autonomously selecting the at least one sample container from the first plurality of sample containers comprises: prioritizing selection of a sample having a confluence score in the second predefined confluence score range and a higher quality score over a sample having a confluence score in the first predefined confluence score range and a lower quality score.

15. The system of claim 1, wherein autonomously performing the one or more maintenance operations on the subset of iPSCs stored within the at least one selected sample container comprises:
performing passaging on the subset of iPSCs stored within the at least one selected sample container, adding one or more reagents to the subset of iPSCs stored within the at least one selected sample container, banking the subset of iPSCs stored within the at least one selected sample container, performing a quality control (QC) check of the subset of iPSCs stored within the at least one selected sample container, performing a pluripotency status check of the subset of iPSCs stored within the at least one selected sample container, or discarding at least the subset of iPSCs stored within the at least one selected sample container.

16. The system of claim 15, wherein discarding at least the subset of iPSCs stored within the at least one selected sample container comprises:
discarding the subset of iPSCs stored within the at least one selected sample container; or
discarding the plurality of iPSCs stored within the first plurality of sample containers.

17. The system of claim 1, wherein autonomously performing the one or more maintenance operations on the subset of iPSCs stored within the at least one selected sample container comprises:
performing passaging of the subset of iPSCs stored within the at least one selected sample container.

18. The system of claim 17, wherein the one or more programs further include instructions for:
distributing, subsequent to the subset of iPSCs stored within the at least one selected sample container being passaged, the subset of iPSCs across a second plurality of sample containers; and
optionally subjecting the distributed subset of iPSCs stored in the second plurality of sample containers to one or more cell differentiation steps.

19. The system of claim 18, wherein subjecting the distributed subset of iPSCs stored in each of the second plurality of sample containers to the one or more cell differentiation steps comprises:
feeding the distributed subset of iPSCs stored in each of the second plurality of sample containers; and
capturing a second plurality of images depicting the distributed subset of iPSCs stored in each of the second plurality of sample containers using the imaging system.

20. The system of claim 19, wherein the one or more programs further include instructions for:
banking the distributed subset of iPSCs stored in the second plurality of sample containers based on a determination that a first predefined amount of time has elapsed from the feeding of the distributed subset of iPSCs stored in each of the second plurality of sample containers.

21. The system of claim 20, wherein the one or more programs further include instructions for:
selecting a sample container from the second plurality of sample containers for performing a QC check.

22. The system of claim 21, wherein the sample container is randomly selected from the second plurality of sample containers.

23. The system of claim 20, wherein the one or more programs further include instructions for:
feeding a subset of iPSCs stored within the selected sample container from the second plurality of sample containers; and
capturing a third plurality of images depicting the iPSCs stored within the selected sample container from the second plurality of sample containers.

24. The system of claim 23, wherein the one or more programs further include instructions for:
determining that a second predefined amount of time has elapsed from the feeding of the iPSCs stored within the selected sample container;
performing the QC check to the iPSCs stored within the selected sample container to obtain a QC score; and
discarding the distributed subset of iPSCs stored in the second plurality of sample containers based on a determination that the QC score is less than a threshold QC score.

25. The system of claim 23, wherein the one or more programs further include instructions for:
subjecting the distributed subset of iPSCs remaining stored within the second plurality of sample containers excluding the iPSCs stored within the selected sample container to one or more additional cell differentiation steps to obtain a plurality of differentiated cells; and
performing one or more phenotypic assessments using at least some of the plurality of differentiated cells.

26. The system of claim 17, wherein passaging comprises:
washing the subset of iPSCs stored within the at least one selected sample container, incubating the washed subset of iPSCs with a dissociation reagent, triturating the incubated subset of iPSCs after a media is added to the incubated subset of iPSCs, transferring the triturated subset of iPSCs to a sample container block, centrifuging the transferred subset of iPSCs in the sample container block to pellet the centrifuged subset of iPSCs, performing a buffering exchange to the pelleted subset of iPSCs by aspirating the pelleted subset of iPSCs, and suspending the aspirated subset of iPSCs into the media.

27. The system of claim 1, wherein the one or more programs further include instructions for:
performing one or more feedings to the plurality of iPSCs stored within the first plurality of sample containers prior to the first plurality of images being captured.

28. The system of claim 1, wherein the one or more programs further include instructions for:
autonomously removing the plurality of iPSCs from cell storage using a cell handling system; and
thawing the plurality of iPSCs using a cell thawing system, wherein the first plurality of images is captured after the thawing.

29. The system of claim 10, wherein the one or more programs further include instructions for:
discarding one or more sample containers from the first plurality of sample containers based on at least one of the quality score, the confluence score, and/or the growth metric of the one or more sample containers.

30. The system of claim 10, wherein the one or more programs further include instructions for:
identifying one or more sample containers, wherein the one or more identified sample containers have at least one of:
a quality score that is (i) less than a first threshold quality score and (ii) greater than or equal to a second threshold quality score,
a confluence score outside of a predefined range of confluence scores, or
a growth metric indicating a growth rate that is negative or being less than a growth metric threshold.

31. The system of claim 30, wherein the one or more programs further include instructions for:
feeding iPSCs stored within the one or more identified sample containers;
capturing a second plurality of images depicting the iPSCs stored within the one or more identified sample containers using the imaging system; and
determining at least one of an updated quality score, an updated confluence score, or an updated growth metric for each of the one or more identified sample containers using the one or more machine learning models.

32. The system of claim 31, wherein the one or more programs further include instructions for:
selecting at least one of the one or more identified sample containers based on the at least one of the updated quality score, the updated confluence score, or the updated growth metric of the one or more sample containers.

33. The system of claim 30, wherein the predefined range of confluence scores is from about 20% to about 90%, from about 25% to about 85%, or from about 30% to about 80%.

34. The system of claim 2, wherein the one or more machine learning models comprise a first machine learning model trained to determine a quality score representing a quality of the iPSCs stored within each of the first plurality of sample containers.

35. The system of claim 33, wherein the one or more machine learning models comprise a second machine learning model trained to determine a confluence score representing a confluence of the iPSCs stored within each of the first plurality of sample containers.

36. The system of claim 34, wherein at least one of the first machine learning model or the second machine learning model comprise a convolutional neural network.

37. The system of claim 34, wherein the first machine learning model is built on a ResNet architecture and the second machine learning model is built on a U-Net architecture.

38. The system of claim 1, wherein the imaging system comprises a digital microscopy imaging system, and wherein the imaging system captures bright-field, phase contrast, or fluorescent images.

39. The system of claim 1, wherein the first plurality of sample containers is disposed on a slide plate, and the slide plate is a multi-well plate comprising a plurality of sample wells.
